(19) United States
(12) Patent Application Publication
(10) Pub. No.: US 2011/0200557 A1
(43) Pub. Date: Aug. 18, 2011

(54) NEW 2-AMIDOTHIADIAZOLE DERIVATIVES

(76) Inventors: Pedro Manuel Grima Poveda, Barcelona (ES); Nuria Aguilar Izquierdo, Barcelona (ES); Marta Mir Cepeda, Barcelona (ES); Manuel Lopez Martinez, Barcelona (ES)

(21) Appl. No.: 13/123,932

(22) PCT Filed: Oct. 13, 2009
(86) PCT No.: PCT/EP09/07348

§ 371 Date: Apr. 27, 2011

(30) Foreign Application Priority Data
Oct. 14, 2008 (EP) 08382042.3

(51) Int. Cl. A61K 38/21; C07D 285/08; A61K 31/433; C07D 417/12; A61K 31/4439; C07D 417/04; C07D 471/04; A61K 31/437; A61K 31/397; A61K 31/454; A61K 39/395; A61K 31/56; A61P 25/28; A61P 37/06; A61P 17/06; A61P 19/02; A61P 11/06

(52) U.S. Cl. 424/85.6; 548/128; 514/361; 546/268.7; 514/342; 546/121; 514/300; 514/210.18; 546/209; 514/326; 424/172.1; 514/171; 424/152.1

(57) ABSTRACT

The present disclosure relates to 2-amidothiadiazole derivatives of formula (I) as well as pharmaceutical compositions comprising them, and their use in therapy as agonists of the S1P1 receptors.

The present invention relates to 2-amidothiadiazole derivatives, to processes for their preparation and to pharmaceutical compositions containing them. These compounds are potent agonists of S1P1 receptors and thus, they are useful in the treatment, prevention or suppression of diseases and disorders known to be susceptible to improvement by sphingosine-1-phosphate receptors agonists (S1P1), such as autoimmune diseases, chronic immune and inflammatory diseases, transplant rejection, malignant neoplastic diseases, angiogenic-related disorders, pain, neurological diseases, viral and infectious diseases.

Sphingosine-1 phosphate (S1P) is a pleiotropic lipid mediator that exhibits a broad spectrum of biological activities, including cell proliferation, survival, lymphocyte trafficking, cytoskeletal organization, and morphogenesis. S1P is generated from endogenous sphingosine through phosphorylation by specific kinases, named sphingosine kinases 1 and 2. The levels of S1P in biological fluids and tissues are tightly regulated by the balance between its synthesis by sphingosine kinases and its degradation by S1P lyase. This tight control is important since an excessive production of S1P has been associated to various pathological conditions, such as angiogenesis and vascular permeability changes in cancer, inflammation, myocardial infarction or transplant rejection.

Gene deletion studies and reverse pharmacology have provided evidence that most of the effects of S1P are mediated via five G-protein coupled receptor subtypes, named S1P1 to S1P5 (Brinkmann, Pharmacology & therapeutics 115:84-105, 2007). The interest on this family of receptors increased following the discovery that they were the pharmacological target of FTY720. This compound, a synthetic analog of a natural product derived from the fungus Isaria sinclairii, exhibited a peculiar immunomodulatory potential in vivo. When administered in vivo, it caused lymphopenia, due to the sequestration of lymphocytes from the blood into the lymph nodes and Peyer's patches. The close structural similarity of FTY720 to sphingosine, together with the discovery of the formation of phosphorylated FTY720 in vivo (FTY720P) prompted to speculate that FTY720-P could be acting as a mimetic of S1P. This proven to be the case and it was later on demonstrated that FTY-P binds 4 of the five known S1P receptors, namely S1P1, S1P3, S1P4 and S1P5.

Expression analysis identified S1P1 as the dominant S1P receptor expressed on lymphocytes. Moreover, the transfer of S1P1-deficient T cells to normal mice led to the cells being sequestered in lymph nodes, as occurred with animals treated with fingolimod. These two facts strongly pointed out at S1P1 as the main receptor involved in the lymphopenic effect of FTY-P in vivo (Baumruker et al, Exp. Opin. Invest. Drugs 2007; 16(3): 283-289). FTY720 is currently in phase III trials for the treatment of relapsing-remitting multiple sclerosis. The drug is presumed to act by causing the retention of pathogenic lymphocytes in lymph nodes, thus preventing them to infiltrate the central nervous system (CNS).

In view of the physiological effects, several S1P1 agonists have been recently disclosed for the treatment or prevention of autoimmune diseases, such as multiple sclerosis (WO2008000419, WO2008021532), rheumatoid arthritis or Crohn's disease (WO2007091501), chronic immune and inflammatory diseases such as asthma, transplant rejection (WO199400943), cancer (WO2003097028), lymphoid malignancies (WO2007143081), angiogenic-related disorders, pain (WO2004110421, WO2007089715) neurological diseases such as neurodegeneration (WO2005025553) or dementia (WO2005058295), cardiovascular diseases (WO2004010987).

Autoimmune diseases include but are not limited to rheumatoid arthritis, multiple sclerosis, inflammatory bowel diseases such as Crohn's diseases and ulcerative colitis, psoriatic arthritis, thyroiditis such as Hashimoto's thyroiditis, type I diabetes; systemic lupus erythematosis and Sjögrn's syndrome.

Rejection transplanted organs such as kidney, liver, heart, lung, pancreas, cornea and skin; graft-versus-hist disease brought about by stem cell transplantation.

Immune and inflammatory diseases which may be prevented or treated include but are not limited to asthma, COPD, respiratory distress syndrome, acute or chronic pancreatitis and hepatitis; chronic sarcoidosis, contact dermatitis, atopic dermatitis, allergic rhinitis, allergic conjunctivitis, Behcet syndrome, inflammatory eye conditions such as conjunctivitis and uveitis.

Malignant neoplastic diseases that may be prevented or treated include but are not limited to solid cancer, tumor metastasis and lymphoid malignancies

Angiogenesis-related disorders that may be prevented or treated include but are not limited to hemangiomas, ocular neovascularization, macular degeneration or diabetic retinopathy.

Pain including neuropathic pain, that may be prevented or treated include but are not limited to prophylaxis or treatment of chronic pain, wherein chronic pain is selected from chronic muscular diseases such as back pain, pain during menstruation, pain during osteoarthritis, pain during rheumatoid arthritis, pain during gastrointestinal inflammation, pain during inflammation of the heart muscle, pain during multiple sclerosis, pain during neuritis, pain during AIDS, pain during chemotherapy, tumor pain, neuropathic pain e.g. after amputation, trigeminal neuralgia, migraine or post herpetic neuralgia.

Cardiovascular diseases which may be prevented or treated include but are not limited to chronic heart failure, congestive heart failure, arrhythmia or tachyarrythmia, unstable angina, acute myocardial infarction or complications from cardiac surgery or for improving heart energy efficiency or cardiac output.

Neurological diseases including neurodegeneration, dementia or brain degeneration that may be prevented or treated include but are not limited to neurological disorders including Parkinson's disease, Parkinsonian disorders, Huntington's disease, Alzheimer's disease, amyotrophic lateral sclerosis, spinal ischemia, ischemic stroke, spinal cord injury, cancer-related brain injury, and cancer-related spinal cord injury, Shy-Drager syndrome, progressive supranuclear palsy, Lewy body disease, stroke, cerebral infarction, multi-infarct dementia, and geriatric dementia,

Viral diseases which may be prevented or treated include but are not limited to HIV infection, hepatitis C and cytomegalovirus infection.

Infectious diseases which may be prevented or treated include but are not limited to pathogenic fungal diseases.

It has now been found that certain 2-amidothiadiazoles are novel and potent agonists of S1P1 and can therefore be used in the treatment or prevention of these diseases.

Thus the present invention is directed to new 2-amidothiadiazole derivatives of formula (I) or pharmaceutically acceptable salts or N-oxides thereof.

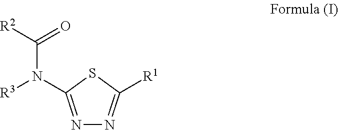

wherein
R¹ represents:
an 8 to 10 membered bicyclic N-containing heteroaryl group optionally substituted by one or more substitutents selected from halogen atoms, hydroxycarbonyl groups, C₁₋₄ alkyl groups, C₁₋₄ haloalkyl groups, C₁₋₄ alkoxy groups and C₃₋₄ cycloalkyl groups;
a pyridyl group substituted with one or more substituents selected from halogen atoms, hydroxy groups, hydroxycarbonyl groups, C₁₋₄ alkyl groups, C₁₋₄ haloalkyl groups, C₁₋₄ alkoxy groups, C₃₋₄ cycloalkyl groups and, —NR′R″ groups, wherein R′ represents a hydrogen atom or a C₁₋₄ alkyl group and R″ represents a hydrogen atom or a C₁₋₄ alkyl group optionally substituted by a hydroxy group;
a pyridinone group substituted with one or more substituents selected from halogen atoms, C₁₋₄ alkyl groups and C₁₋₄ haloalkyl groups; or
a group of formula:

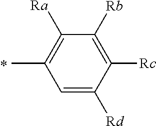

wherein:
Ra represents a hydrogen atom or a C₁₋₄ alkyl group,
Rb represents a hydrogen atom, halogen atom or C₁₋₄ alkyl group,
Rd represents a hydrogen atom, a C₁₋₄ alkyl group or a C₃₋₄ cycloalkyl group,
Rc represents a hydroxy group; a C₁₋₄ alkoxy group which is optionally substituted with one or more substituents selected from hydroxy groups, C₁₋₃ alkoxy groups, hydroxycarbonyl groups, C₁₋₄ alkoxycarbonyl groups and NHR⁴ groups, wherein R⁴ represents a hydrogen atom; or Rc is a C₂₋₄ acyl group or a C₁₋₄ alkyl group optionally substituted by a hydroxycarbonyl group, or Rc represents —(CH₂)₍₀₋₄₎-L-R⁵ wherein L represents —C(O)O—, —C(O)NH—, —S(O)₂NH—, —NH—, —CONHS(O)₂— or a group of formula:

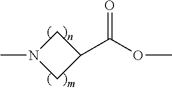

wherein n and m independently are integer from 1 to 2, and R⁵ represents a hydrogen atom or a C₁₋₄ alkyl group optionally substituted by a hydroxycarbonyl group;
R² represents:
a monocyclic or bicyclic C₅₋₁₀ aryl group optionally substituted with one or more substituents selected from halogen atoms, C₁₋₄ alkyl groups, C₁₋₄ alkoxy groups and phenyl groups, wherein the alkyl group is optionally substituted by one or more halogen atoms,
a monocyclic or bicyclic 5-10 membered heteroaryl group comprising one or more heteroatoms selected from N, S and O optionally substituted with one or more substituents selected from halogen atoms, C₁₋₄ alkyl and C₁₋₄ alkoxy groups, wherein the C₁₋₄ alkyl group is optionally substituted by one or more halogen atoms,
a dihydrobenzodioxine group or a benzyl group which is optionally substituted with one or more substituents selected from halogen atoms,
   and
   R³ represents:
a linear or branched C₁₋₆ alkyl, C₃₋₆ cycloalkyl-C₁₋₄ alkyl, C₁₋₄ alkoxy-C₁₋₄ alkyl, di-alkylamino-C₁₋₄alkyl group, or phenyl-C₁₋₄alkyl group;

With the proviso that when Rc represents a methoxy or ethoxy group, then one of Rb or Rd can not be a hydrogen atom;

With the additional proviso that the compound of formula (I) is not N-methyl-N-(5-(6-methylpyridin-3-yl)-1,3,4-thiadiazol-2-yl)nicotinamide, nor N-methyl-N-(5-(6-methylpyridin-3-yl)-1,3,4-thiadiazol-2-yl)isonicotinamide.

Further objectives of the present invention are to provide a method for preparing said compounds; pharmaceutical compositions comprising an effective amount of said compounds; the use of the compounds in the manufacture of a medicament for the treatment of pathological conditions or diseases susceptible to improvement by sphingosine-1-phosphate receptors agonists (S1P1), wherein the pathological condition or disease is selected from autoimmune diseases, chronic immune and inflammatory diseases, transplant rejection, malignant neoplastic diseases, angiogenic-related disorders, pain, neurological diseases, viral and infectious diseases, and methods of treatment of pathological conditions or diseases susceptible to amelioration by sphingosine-1-phosphate receptors agonists (S1P1), wherein the pathological condition or disease is selected from autoimmune diseases, chronic immune and inflammatory diseases, transplant rejection, malignant neoplastic diseases, angiogenic-related disorders, pain, neurological diseases, viral and infectious diseases comprising the administration of the compounds of the invention to a subject in need of treatment.

As used herein the term alkyl embraces optionally substituted, linear or branched hydrocarbon radicals having 1 to 6 carbon atoms, preferably from 1 to 4 carbon atoms. Preferred substituents on the alkyl groups are halogen atoms and hydroxy groups, and are more preferably halogen atoms.

Examples include methyl, ethyl, n-propyl, i-propyl, n-butyl, n-pentyl, n-hexyl, i-butyl, sec-butyl and tert-butyl radicals.

As used herein, a haloalkyl group is a said alkyl group, for example a C₁₋₄ or C₁₋₂ alkyl group, which is attached to 1, 2 or 3 halogen atoms.

Preferably, said haloalkyl group is chosen from —CCl₃ and —CF₃.

As used herein the term alkoxy embraces optionally substituted, linear or branched oxy-containing radicals each having 1 to 4 carbon atoms. Preferred substituents on the alkoxy groups are halogen atoms and hydroxy groups, and are more preferably halogen atoms.

Examples include methoxy, ethoxy, n-propoxy, i-propoxy, n-butoxy, sec-butoxy and tert-butoxy radicals.

As used herein, the term cycloalkyl embraces saturated carbocyclic radicals and, unless otherwise specified, a cycloalkyl radical typically has from 3 to 6 carbon atoms, preferably from 3 to 4 carbon atoms.

Examples include cyclopropyl, cyclobutyl, cyclopentyl and cyclohexyl. When a cycloalkyl radical carries 2 or more substituents, the substituents may be the same or different. Preferred substituents on the cycloalkyl groups are halogen atoms and hydroxy groups, and are more preferably halogen atoms.

As used herein, the term dialkylamino embraces radicals containing a trivalent nitrogen atoms with two optionally substituted, linear or branched alkyl radicals of 1 to 4 carbon atoms in each alkyl radical.

A dialkylamino group typically contains two alkyl groups, each of which is unsubstituted or substituted with 1, 2 or 3 substituents which may be the same or different. The substituents are preferably selected from halogen atoms, preferably fluorine atoms, hydroxy groups and alkoxy groups having from 1 to 4 carbon atoms. Typically, the substituents on a dialkylamino group are themselves unsubstituted.

Preferred optionally substituted dialkylamino radicals include dimethylamino, diethylamino, methyl(ethyl)amino, di(n-propyl)amino, n-propyl(methyl)amino, n-propyl(ethyl)amino, di(i-propyl)amino, i-propyl(methyl)amino, i-propyl(ethyl)amino, di(n-butyl)amino, n-butyl(methyl)amino, n-butyl(ethyl)amino, n-butyl(i-propyl)amino, di(sec-butyl)amino, sec-butyl(methyl)amino, sec-butyl(ethyl)amino, sec-butyl(n-propyl)amino and sec-butyl(i-propyl)amino.

As used herein, the term alkoxycarbonyl embraces optionally substituted, linear or branched radicals each having alkyl portions of 1 to 4 carbon atoms.

An alkoxycarbonyl group is typically unsubstituted or substituted with 1, 2 or 3 substituents which may be the same or different. The substituents are preferably selected from halogen atoms, preferably fluorine atoms, hydroxy groups and alkoxy groups having from 1 to 4 carbon atoms. Typically, the substituents on an alkoxycarbonyl group are themselves unsubstituted.

Preferred optionally substituted alkoxycarbonyl radicals include methoxycarbonyl, ethoxycarbonyl, n-propoxycarbonyl, i-propoxycarbonyl, n-butoxycarbonyl, sec-butoxycarbonyl, trifluoromethoxycarbonyl, difluoromethoxycarbonyl, hydroxymethoxycarbonyl, 2-hydroxyethoxycarbonyl and 2-hydroxypropoxycarbonyl.

As used herein, the term acyl embraces optionally substituted, linear or branched radicals having 2 to 4 carbon atoms.

An acyl group is typically unsubstituted or substituted with 1, 2 or 3 substituents which may be the same or different. The substituents are preferably selected from halogen atoms, preferably fluorine atoms, hydroxy groups and alkoxy groups having from 1 to 4 carbon atoms. Typically, the substituents on an acyl group are themselves unsubstituted.

Preferred optionally substituted acyl radicals include acetyl, propionyl and butyryl,

As used herein, the term aryl radical embraces typically a C₅-C₁₀ monocyclic or polycyclic aryl radical such as phenyl and naphthyl. C₆-C₁₀ aryl groups are preferred. Phenyl is more preferred.

A said optionally substituted aryl radical is typically unsubstituted or substituted with 1, 2 or 3 substituents which may be the same or different. The substituents are preferably selected from halogen atoms, preferably fluorine atoms, hydroxy groups, alkoxycarbonyl groups in which the alkyl moiety has from 1 to 4 carbon atoms, hydroxycarbonyl groups, carbamoyl groups, nitro groups, cyano groups, C₁-C₄ alkyl groups, C₁-C₄ alkoxy groups and C₁-C₄ hydroxyalkyl groups. When an aryl radical carries 2 or more substituents, the substituents may be the same or different. Unless otherwise specified, the substituents on an aryl group are typically themselves unsubstituted.

As used herein, the term heteroaryl radical embraces typically a 5- to 10-membered ring system comprising at least one heteroaromatic ring and containing at least one heteroatom selected from O, S and N. A heteroaryl radical may be a single ring or two or more fused rings wherein at least one ring contains a heteroatom.

A said optionally substituted heteroaryl radical is typically unsubstituted or substituted with 1, 2 or 3 substituents which may be the same or different. The substituents are preferably selected from halogen atoms, preferably fluorine, chlorine or bromine atoms, alkoxycarbonyl groups in which the alkyl moiety has from 1 to 4 carbon atoms, nitro groups, hydroxy groups, C₁-C₄ alkyl groups and C₁-C₄ alkoxy groups. When an heteroaryl radical carries 2 or more substituents, the substituents may be the same or different. Unless otherwise specified, the substituents on a heteroaryl radical are typically themselves unsubstituted.

Examples include pyridyl, pyrazinyl, pyrimidinyl, pyridazinyl, furyl, benzofuranyl, oxadiazolyl, oxazolyl, isoxazolyl, benzoxazolyl, imidazolyl, benzimidazolyl, thiazolyl, thiadiazolyl, thienyl, pyrrolyl, pyridinyl, benzothiazolyl, indolyl, indazolyl, purinyl, quinolyl, isoquinolyl, phthalazinyl, naphthyridinyl, quinoxalinyl, quinazolinyl, quinolizinyl, cinnolinyl, triazolyl, indolizinyl, indolinyl, isoindolinyl, isoindolyl, imidazolidinyl, pteridinyl, thianthrenyl, pyrazolyl, 2H-pyrazolo[3,4-d]pyrimidinyl, 1H-pyrazolo[3,4-d]pyrimidinyl, thieno[2,3-d]pyrimidinyl and the various pyrrolopyridyl radicals.

Oxadiazolyl, oxazolyl, pyridyl, pyrrolyl, imidazolyl, thiazolyl, thiadiazolyl, thienyl, furanyl, quinolinyl, isoquinolinyl, indolyl, benzoxazolyl, naphthyridinyl, benzofuranyl, pyrazinyl, pyrimidinyl and the various pyrrolopyridyl radicals are preferred.

As used herein, some of the atoms, radicals, moieties, chains or cycles present in the general structures of the invention are “optionally substituted”. This means that these atoms, radicals, moieties, chains or cycles can be either unsubstituted or substituted in any position by one or more, for example 1, 2, 3 or 4, substituents, whereby the hydrogen atoms bound to the unsubstituted atoms, radicals, moieties, chains or cycles are replaced by chemically acceptable atoms, radicals, moieties, chains or cycles. When two or more substituents are present, each substituent may be the same or different.

As used herein, the term halogen atom embraces chlorine, fluorine, bromine or iodine atoms typically a fluorine, chlorine or bromine atom, most preferably bromine or fluorine. The term halo when used as a prefix has the same meaning.

As used herein, the term pharmaceutically acceptable salt embraces salts with a pharmaceutically acceptable acid or base. Pharmaceutically acceptable acids include both inorganic acids, for example hydrochloric, sulphuric, phosphoric, diphosphoric, hydrobromic, hydroiodic and nitric acid and organic acids, for example citric, fumaric, maleic, malic, mandelic, ascorbic, oxalic, succinic, tartaric, benzoic, acetic, methanesulphonic, ethanesulphonic, benzenesulphonic or p-toluenesulphonic acid. Pharmaceutically acceptable bases include alkali metal (e.g. sodium or potassium) and alkali earth metal (e.g. calcium or magnesium) hydroxides and organic bases, for example alkyl amines, arylalkyl amines and heterocyclic amines.

Other preferred salts according to the invention are quaternary ammonium compounds wherein an equivalent of an anion (X—) is associated with the positive charge of the N atom. X— may be an anion of various mineral acids such as, for example, chloride, bromide, iodide, sulphate, nitrate, phosphate, or an anion of an organic acid such as, for example, acetate, maleate, fumarate, citrate, oxalate, succinate, tartrate, malate, mandelate, trifluoroacetate, methanesulphonate and p-toluenesulphonate. X— is preferably an anion selected from chloride, bromide, iodide, sulphate, nitrate, acetate, maleate, oxalate, succinate or trifluoroacetate. More preferably X— is chloride, bromide, trifluoroacetate or methanesulphonate.

Typically, in the definition of R¹, C₁₋₄ alkyl group, C₁₋₄ haloalkyl group, C₁₋₄ alkoxy group and C₃₋₄ cycloalkyl group substituents on 8 to 10 membered bicyclic N-containing heteroaryl groups or pyridyl groups are themselves unsubstituted.

Typically, in the definition of R¹, C₁₋₄ alkyl group and C₁₋₄ haloalkyl group substituents on pyridinone groups are themselves unsubstituted.

Typically, in the definition of R¹, when Rc represents a C₁₋₄ alkoxy group, C₁₋₃ alkoxy group, and C₁₋₄ alkoxycarbonyl group substituents on said C₁₋₄ alkoxy group are themselves unsubstituted.

Typically, in the definition of R², C₁₋₄ alkoxy group and phenyl group substituents on monocyclic or bicyclic C₅₋₁₀ aryl groups or monocyclic or bicyclic 5-10 membered heteroaryl group comprising one or more heteroatoms selected from N, S and O are themselves unsubstituted.

As used herein, an N-oxide is formed from the tertiary basic amines or imines present in the molecule, using a convenient oxidising agent.

Typically, R⁵ represents a hydrogen atom or a linear or branched C₁₋₄ alkyl group optionally substituted by a hydroxycarbonyl group.

Typically, R¹ represents an imidazo[1,2-a]pyridyl group; a pyridyl group which is substituted with one or more substituents selected from halogen atoms, C₁₋₄ alkyl groups and C₁₋₄ alkoxy groups; a pyridinone group substituted with a chlorine atom; or a group of formula:

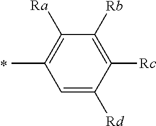

wherein:
Ra represents a hydrogen atom or a methyl group,
Rb represents a hydrogen atom, halogen atom or C₁₋₄ alkyl group,
Rd represents a hydrogen atom or a C₁₋₄ alkyl group,
Rc represents a hydroxy group, a C₁₋₄ alkoxy group which is optionally substituted with one or more substituents selected from hydroxy groups or C₁₋₃ alkoxy groups, or Rc represents —(CH₂)₍₀₋₂₎-L-R⁵, wherein L represents —C(O)NH—, —NH—, or a group of formula:

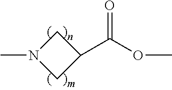

wherein n and m independently are integers from 1 to 2, and R⁵ represents a hydrogen atom or a C₁₋₄ alkyl group optionally substituted by a hydroxycarbonyl group.

Preferably, R¹ represents a pyridyl group substituted with one or two substituents selected from halogen atoms, C₁₋₄ alkyl groups and C₁₋₄ alkoxy groups; or a group of formula:

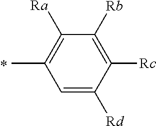

wherein:
Ra represents a hydrogen atom,
Rb represents a hydrogen atom or a C₁₋₄ alkyl group,
Rd represents a hydrogen atom or C₁₋₄ alkyl group,
Rc represents a hydroxy group, a C₁₋₄ alkoxy group which is optionally substituted by one or more substituents selected from hydroxy groups, or Rc represents —(CH₂)₍₀₋₂₎-L-R⁵, wherein L represents —C(O)NH—, —NH—, or a group of formula:

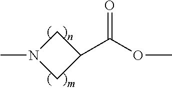

wherein n and m independently are integers from 1 to 2, and R⁵ represents a hydrogen atom or a C₁₋₄ alkyl group optionally substituted by a hydroxycarbonyl group.

More preferably, R¹ represents a pyridyl group substituted with one or two substituents selected from chlorine atoms, methyl and methoxy groups; or a group of formula:

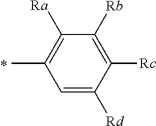

wherein:
Ra represents a hydrogen atom,
Rd represents a hydrogen atom or methyl group,
Rb represents a hydrogen atom or methyl group,
Rc represents a hydroxy group, a C₁₋₄ alkoxy group which is optionally substituted by one or more substituents selected from hydroxy groups; or Rc represents —(CH₂)₍₀₋₁₎-L-R⁵, wherein L represents —C(O)NH—, —NH—, or a group of formula:

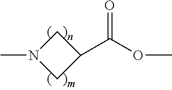

wherein both n and m have a value of 1, and R⁵ represents a hydrogen atom or a C₁₋₂ alkyl group optionally substituted by a hydroxycarbonyl group.

Typically, R² represents a phenyl group substituted by one substituent selected from a halogen atom, a C₁₋₄ alkyl group and a C₁₋₄ alkoxy group, wherein the alkyl group is optionally substituted by one or more halogen atoms; or R² represents a monocyclic N-containing 5-10 membered heteroaryl group which is substituted by a halogen atom.

Preferably, R² represents a phenyl group which is substituted with one substituent selected from a fluorine atom, a chlorine atom, a methyl, a trifluoromethyl or a methoxy group; or R² represents a pyridyl group which is substituted with a fluorine atom.

More preferably, R² represents a phenyl group substituted with one substituent selected from a fluorine atom, a chlorine atom or a methoxy group.

Typically, R³ represents a linear C₃₋₆ alkyl group or a C₃₋₄ cycloalkyl-C₁₋₂ alkyl group.

Preferably, R³ represents a propyl, butyl or cyclopropylmethyl group.

More preferably, R³ represents a butyl or a cyclopropylmethyl group.

Most preferably, R¹ represents a pyridyl group substituted with one or two substituents selected from chlorine atoms, methyl and methoxy groups; or a group of formula:

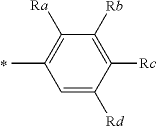

wherein:
Ra represents a hydrogen atom,
Rb represents a hydrogen atom or methyl group,
Rd represents a hydrogen atom or methyl group,
Rc represents a hydroxy group, a C₁₋₄ alkoxy group which is optionally substituted with one or more substituents selected from hydroxy groups; or Rc represents —(CH₂)₍₀₋₁₎-L-R⁵, wherein L represents —C(O)NH—, —NH—, or a group of formula:

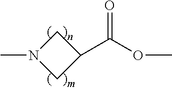

wherein both n and m have a value of 1, and R⁵ represents a hydrogen atom or a C₁₋₄ alkyl group optionally substituted by a hydroxycarbonyl group;

R² represents a phenyl group substituted with one substituent selected from a fluorine atom, a chlorine atom and a methoxy group;

and R³ represents a butyl or a cyclopropylmethyl group.

In a preferred embodiment, R¹ represents an imidazo[1,2-a]pyridyl group; a pyridyl group substituted with one or two substituents selected from chlorine atoms, methyl groups and methoxy groups; pyridone group substituted with a chlorine atom; or a group of formula:

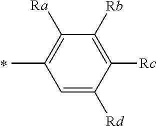

wherein
Ra represents a hydrogen atom or a methyl group,
Rb represents a hydrogen atom, a chlorine atom or a methyl group,
Rd represents a hydrogen atom or a methyl group,
Rc represents a hydroxy group; a C₁₋₄ alkoxy group which is optionally substituted with one or more substituents selected from hydroxy groups, methoxy groups, hydroxycarbonyl groups, C₁₋₄ alkoxycarbonyl groups; or Rc represents —(CH₂)₍₀₋₂₎-L-R⁵, wherein L represents —CO(O)—, —C(O)NH—, —S(O)₂NH— or a group of formula:

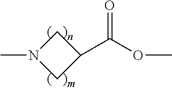

wherein n and m are both 1, and R⁵ represents a hydrogen atom or a C₁₋₂ alkyl group optionally substituted by a hydroxycarbonyl group;
R² represents:
a phenyl or naphthyl group optionally substituted with one or two substituents selected from chlorine atoms, fluorine atoms, methyl groups, trifluoromethyl groups, C₁₋₄ alkoxy groups and phenyl groups;
a pyridine group optionally substituted with a fluorine atom;
a dihydrobenzodioxine group or a benzyl group; and
   R³ represents a linear or branched C₁₋₅ alkyl, cyclopropylmethyl, methoxypropyl, diethylaminopropyl or phenylethyl group.

In another preferred embodiment, R¹ represents a group of formula:

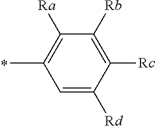

wherein:
Ra represents a hydrogen atom,
Rb represents a hydrogen atom or a C₁₋₄ alkyl group,
Rd represents a hydrogen atom or a C₁₋₄ alkyl group,
Rc represents a C₁₋₄ alkoxy group which is optionally substituted with one or more substituents selected from hydroxy groups or —NH₂ groups, or Rc represents —(CH₂)₍₀₋₁₎-L-R⁵, wherein L represents a group of formula:

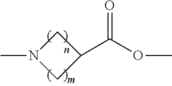

wherein both n and m have a value of 2, and R⁵ represents a hydrogen atom or a C₁₋₄ alkyl group optionally substituted by a hydroxycarbonyl group;
R² represents a phenyl group substituted with one or two substituents selected from a fluorine or chlorine atoms; and
R³ represents a butyl or a cyclopropylmethyl group.

In another preferred embodiment, R¹ represents:
an imidazo[1,2-a]pyridyl group;
a pyridyl group substituted with one or two substituents selected from chlorine atoms, methyl groups and methoxy groups;
a pyridone group optionally substituted with a chlorine atom or a methyl group; or
a group of formula:

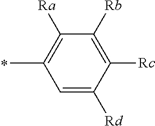

wherein
Ra represents a hydrogen atom or a methyl group,
Rb represents a hydrogen atom, a chlorine atom or a methyl group,
Rd represents a hydrogen atom or a methyl group,
Rc represents a hydroxy group; a C₁₋₄ alkoxy group which is optionally substituted with one or more substituents selected from hydroxy groups, methoxy groups, hydroxycarbonyl groups, C₁₋₄ alkoxycarbonyl groups and —NH₂ groups; or Rc represents —(CH₂)₍₀₋₂₎-L-R⁵, wherein L represents —CO(O)—, —C(O)NH—, —S(O)₂NH—, —NH— or a group of formula:

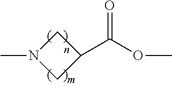

wherein n and m independently are integers of 1 or 2, and R⁵ represents a hydrogen atom or a linear or branched C₁₋₃ alkyl group optionally substituted by a hydroxycarbonyl group;
R² represents:
a phenyl or naphthyl group optionally substituted with one or two substituents selected from chlorine atoms, fluorine atoms, methyl groups, trifluoromethyl groups, C₁₋₄ alkoxy groups and phenyl groups;
a pyridine group optionally substituted with a fluorine atom;
a dihydrobenzodioxine group or a benzyl group; and
   R³ represents a linear or branched C₁₋₅ alkyl, cyclopropylmethyl, methoxypropyl, diethylaminopropyl or phenylethyl group.

Particular individual compounds of the invention include:
N-{5-[4-(aminosulfonyl)phenyl]-1,3,4-thiadiazol-2-yl}-N-butyl-3-methoxy-benzamide
N-{5-[4-(aminosulfonyl)phenyl]-1,3,4-thiadiazol-2-yl}-N-butyl-2-naphthamide
N-{5-[4-(aminosulfonyl)phenyl]-1,3,4-thiadiazol-2-yl}-N-butylbiphenyl-4-carboxamide
N-{5-[4-(aminosulfonyl)phenyl]-1,3,4-thiadiazol-2-yl}-4-butoxy-N-butylbenzamide
N-{5-[4-(aminosulfonyl)phenyl]-1,3,4-thiadiazol-2-yl}-N-butylbenzamide
3-(4-(5-(N-butyl-3-methoxybenzamido)-1,3,4-thiadiazol-2-yl)phenyl)propanoic acid
N-butyl-2-chloro-N-[5-(4-methoxy-3,5-dimethylphenyl)-1,3,4-thiadiazol-2-yl]benzamide
N-butyl-2-chloro-N-[5-(4-hydroxy-3,5-dimethylphenyl)-1,3,4-thiadiazol-2-yl]benzamide
N-butyl-2-fluoro-N-[5-(4-methoxy-3,5-dimethylphenyl)-1,3,4-thiadiazol-2-yl]benzamide
N-Butyl-2-fluoro-N-[5-(4-hydroxy-3,5-dimethylphenyl)-1,3,4-thiadiazol-2-yl]benzamide
N-butyl-N-[5-(4-methoxy-3,5-dimethylphenyl)-1,3,4-thiadiazol-2-yl]benzamide
N-butyl-3-methoxy-N-[5-(4-methoxy-3,5-dimethylphenyl)-1,3,4-thiadiazol-2-yl]benzamide
N-butyl-N-[5-(4-methoxy-3,5-dimethylphenyl)-1,3,4-thiadiazol-2-yl]-1-naphthamide
N-butyl-2,6-dichloro-N-(5-(4-methoxy-3,5-dimethylphenyl)-1,3,4-thiadiazol-2-yl)benzamide
N-butyl-N-[5-(4-methoxy-3,5-dimethylphenyl)-1,3,4-thiadiazol-2-yl]-2-(trifluoromethyl)benzamide
N-butyl-N-[5-(4-methoxy-3,5-dimethylphenyl)-1,3,4-thiadiazol-2-yl]-2-phenylacetamide
N-butyl-N-[5-(4-methoxy-3,5-dimethylphenyl)-1,3,4-thiadiazol-2-yl]-2-naphthamide
N-butyl-N-[5-(4-methoxy-3,5-dimethylphenyl)-1,3,4-thiadiazol-2-yl]-2,3-dihydro-1,4-benzodioxine-6-carboxamide
N-butyl-2-methoxy-N-[5-(4-methoxy-3,5-dimethylphenyl)-1,3,4-thiadiazol-2-yl]benzamide
N-Butyl-3-fluoro-N-[5-(4-methoxy-3,5-dimethylphenyl)-1,3,4-thiadiazol-2-yl]benzamide
N-Butyl-N-[5-(4-methoxy-3,5-dimethylphenyl)-1,3,4-thiadiazol-2-yl]nicotinamide
N-Butyl-N-[5-(4-methoxy-3,5-dimethylphenyl)-1,3,4-thiadiazol-2-yl]pyridine-2-carboxamide
N-Butyl-6-fluoro-N-[5-(4-methoxy-3,5-dimethylphenyl)-1,3,4-thiadiazol-2-yl]pyridine-2-carboxamide
N-butyl-N-[5-(4-methoxy-3,5-dimethylphenyl)-1,3,4-thiadiazol-2-yl]-2-methylbenzamide
2-chloro-N-[5-(4-methoxy-3,5-dimethylphenyl)-1,3,4-thiadiazol-2-yl]-Nmethylbenzamide
N-butyl-2-chloro-N-[5-(4-methoxy-3-methylphenyl)-1,3,4-thiadiazol-2-yl]benzamide
N-butyl-2-chloro-N-[5-(3-chloro-4-methoxyphenyl)-1,3,4-thiadiazol-2-yl]benzamide
Methyl 4-{5-[butyl(2-chlorobenzoyl)amino]-1,3,4-thiadiazol-2-yl}benzoate
4-{5-[Butyl(2-chlorobenzoyl)amino]-1,3,4-thiadiazol-2-yl}benzoic acid
N-Butyl-2-chloro-N-{5-[4-(2,3-dihydroxypropoxy)-3,5-dimethylphenyl]-1,3,4-thiadiazol-2-yl}benzamide
N-Butyl-2-chloro-N-[5-(2-chloro-6-methoxypyridin-4-yl)-1,3,4-thiadiazol-2-yl]benzamide
N-butyl-2-chloro-N-{5-[4-(2-methoxyethoxy)-3,5-dimethylphenyl]-1,3,4-thiadiazol-2-yl}benzamide
2-Chloro-N-[5-(4-methoxy-3,5-dimethylphenyl)-1,3,4-thiadiazol-2-yl]-N-(2-methoxyethyl)benzamide
2-Chloro-N-ethyl-N-[5-(4-methoxy-3,5-dimethylphenyl)-1,3,4-thiadiazol-2-yl]benzamide
tert-Butyl (4-{5-[butyl(2-chlorobenzoyl)amino]-1,3,4-thiadiazol-2-yl}-2,6-dimethyl-phenoxy)acetate
(4-{5-[Butyl(2-chlorobenzoyl)amino]-1,3,4-thiadiazol-2-yl}-2,6-dimethylphenoxy)acetic acid
2-Chloro-N-[5-(4-methoxy-3,5-dimethylphenyl)-1,3,4-thiadiazol-2-yl]-N-(3-methylbutyl)benzamide
2-Chloro-N-[3-(diethylamino)propyl]-N-[5-(4-methoxy-3,5-dimethylphenyl)-1,3,4-thiadiazol-2-yl]benzamide
N-Butyl-2-chloro-N-[5-(2-chloro-6-methylpyridin-4-yl)-1,3,4-thiadiazol-2-yl]benzamide
N-Butyl-2-chloro-N-[5-(6-chloro-2-oxo-1,2-dihydropyridin-4-yl)-1,3,4-thiadiazol-2-yl]benzamide
N-Butyl-N-[5-(2-chloro-6-methoxypyridin-4-yl)-1,3,4-thiadiazol-2-yl]-2-fluorobenzamide
N-Butyl-N-[5-(2-chloro-6-methylpyridin-4-yl)-1,3,4-thiadiazol-2-yl]-2-fluorobenzamide
2-fluoro-N-[5-(4-methoxy-3,5-dimethylphenyl)-1,3,4-thiadiazol-2-yl]-N-propylbenzamide
N-Butyl-2-fluoro-N-[5-(2-methylpyridin-4-yl)-1,3,4-thiadiazol-2-yl]benzamide
N-butyl-2-fluoro-N-[5-(2-methoxypyridin-4-yl)-1,3,4-thiadiazol-2-yl]benzamide
N-(cyclopropylmethyl)-2-fluoro-N-[5-(4-methoxy-3,5-dimethylphenyl)-1,3,4-thiadiazol-2-yl]benzamide
3-(4-{5-[butyl(2-fluorobenzoyl)amino]-1,3,4-thiadiazol-2-yl}-2,6-dimethyl-phenyl)propanoic acid
N-butyl-2-fluoro-N-(5-(6-methoxypyridin-3-yl)-1,3,4-thiadiazol-2-yl)benzamide
N-butyl-2-fluoro-N-(5-(imidazo[1,2-a]pyridin-6-yl)-1,3,4-thiadiazol-2-yl)benzamide
N-butyl-2-fluoro-N-(5-(imidazo[1,2-a]pyridin-7-yl)-1,3,4-thiadiazol-2-yl)benzamide
3-(4-(5-(N-butyl-2-chlorobenzamido)-1,3,4-thiadiazol-2-yl)benzamido) propanoic acid
Ethyl 3-(4-(5-(N-butyl-2-chlorobenzamido)-1,3,4-thiadiazol-2-yl)benzamido) propanoate
N-Butyl-N-(5-(4-carbamoylphenyl)-1,3,4-thiadiazol-2-yl)-2-chlorobenzamide
1-(4-(5-(N-butyl-2-fluorobenzamido)-1,3,4-thiadiazol-2-yl)benzyl)azetidine-3-carboxylic acid
(R)—N-Butyl-N-(5-(4-(2,3-dihydroxypropoxy)-3,5-dimethylphenyl)-1,3,4-thiadiazol-2-yl)-2-fluorobenzamide
2-Fluoro-N-(5-(4-methoxy-3,5-dimethylphenyl)-1,3,4-thiadiazol-2-yl)-N-phenethylbenzamide
2-(4-(5-(N-Butyl-2-fluorobenzamido)-1,3,4-thiadiazol-2-yl)benzamido)acetic acid
N-Butyl-2-fluoro-N-[5-(1-methyl-2-oxo-1,2-dihydropyridin-4-yl)-1,3,4-thiadiazol-2-yl]benzamide
N-(4-{5-[Butyl(2-fluorobenzoyl)amino]-1,3,4-thiadiazol-2-yl}benzyl)beta-alanine
N-Butyl-2-fluoro-N-[5-(1-methyl-6-oxo-1,6-dihydropyridin-3-yl)-1,3,4-thiadiazol-2-yl]benzamide
N-Butyl-2-fluoro-N-[5-(6-oxo-1,6-dihydropyridin-3-yl)-1,3,4-thiadiazol-2-yl]benzamide
3-(4-{5-[Ethyl(2-fluorobenzoyl)amino]-1,3,4-thiadiazol-2-yl}-2,6-dimethylphenyl)propanoic acid
N-Butyl-N-[5-(4-{[(2S)-2,3-dihydroxypropyl]oxy}-3,5-dimethylphenyl)-1,3,4-thiadiazol-2-yl]-2-fluorobenzamide
1-(4-{5-[Butyl(2-fluorobenzoyl)amino]-1,3,4-thiadiazol-2-yl}benzyl)piperidine-4-carboxylic acid
1-(4-{5-[Butyl(2-methoxybenzoyl)amino]-1,3,4-thiadiazol-2-yl}benzyl)azetidine-3-carboxylic acid
N-(Cyclopropylmethyl)-N-[5-(4-{[(2R)-2,3-dihydroxypropyl]oxy}-3,5-dimethylphenyl)-1,3,4-thiadiazol-2-yl]-2-fluorobenzamide
1-(4-{5-[Butyl(pyridin-3-ylcarbonyl)amino]-1,3,4-thiadiazol-2-yl}benzyl)azetidine-3-carboxylic acid
1-(4-{5-[Ethyl(2-fluorobenzoyl)amino]-1,3,4-thiadiazol-2-yl}benzyl)azetidine-3-carboxylic acid
N-(4-{5-[Butyl(2-fluorobenzoyl)amino]-1,3,4-thiadiazol-2-yl}benzoyl)-beta-alanine
N-(Cyclopropylmethyl)-N-[5-(4-{[(2S)-2,3-dihydroxypropyl]oxy}-3,5-dimethylphenyl)-1,3,4-thiadiazol-2-yl]-2-fluorobenzamide
1-(4-{5-[(2-Fluorobenzoyl)(propyl)amino]-1,3,4-thiadiazol-2-yl}benzyl)azetidine-3-carboxylic acid
(3R)-3-[(4-{5-[Butyl(2-fluorobenzoyl)amino]-1,3,4-thiadiazol-2-yl}benzoyl)amino]-butanoic acid
(3S)-3-[(4-{5-[Butyl(2-fluorobenzoyl)amino]-1,3,4-thiadiazol-2-yl}benzoyl)amino]-butanoic acid
N-{5-[4-(Aminomethyl)phenyl]-1,3,4-thiadiazol-2-yl}-N-butyl-2-fluorobenzamide
1-[2-(4-{5-[Butyl(2-fluorobenzoyl)amino]-1,3,4-thiadiazol-2-yl}phenyl)ethyl]azetidine-3-carboxylic acid
1-(4-{5-[(Cyclopropylmethyl)(2-fluorobenzoyl)amino]-1,3,4-thiadiazol-2-yl}benzyl)-azetidine-3-carboxylic acid
1-(4-{5-[(2-Fluorobenzoyl)(3-methylbutyl)amino]-1,3,4-thiadiazol-2-yl}benzyl)-azetidine-3-carboxylic acid
1-(4-{5-[(2-Fluorobenzoyl)(methyl)amino]-1,3,4-thiadiazol-2-yl}benzyl)azetidine-3-carboxylic acid
1-(4-{5-[Butyl(2-fluorobenzoyl)amino]-1,3,4-thiadiazol-2-yl}-3-methylbenzyl)-azetidine-3-carboxylic acid
4-[(4-{5-[Butyl(2-fluorobenzoyl)amino]-1,3,4-thiadiazol-2-yl}benzoyl)amino]butanoic acid
1-(4-{5-[(2-Fluorobenzoyl)(2-methoxyethyl)amino]-1,3,4-thiadiazol-2-yl}benzyl)-azetidine-3-carboxylic acid
1-(4-{5-[Butyl(phenylacetyl)amino]-1,3,4-thiadiazol-2-yl}benzyl)azetidine-3-carboxylic acid
1-(4-{5-[Butyl(2,6-difluorobenzoyl)amino]-1,3,4-thiadiazol-2-yl}benzyl)azetidine-3-carboxylic acid
N-(4-{5-[Butyl(phenylacetyl)amino]-1,3,4-thiadiazol-2-yl}benzyl)beta-alanine
N-(4-{5-[Butyl(2-fluorobenzoyl)amino]-1,3,4-thiadiazol-2-yl}benzyl)glycine
ethyl 1-(4-{5-[Butyl(2-fluorobenzoyl)amino]-1,3,4-thiadiazol-2-yl}benzyl)azetidine-3-carboxylate
1-[4-(5-{Butyl[(2-fluorophenyl)acetyl]amino}-1,3,4-thiadiazol-2-yl)benzyl]azetidine-3-carboxylic acid
1-(4-{5-[(Cyclopropylmethyl)(2-methylbenzoyl)amino]-1,3,4-thiadiazol-2-yl}benzyl)-azetidine-3-carboxylic acid
1-(4-{5-[(Cyclopropylmethyl)(phenylacetyl)amino]-1,3,4-thiadiazol-2-yl}benzyl)-azetidine-3-carboxylic acid
1-(4-{5-[(Cyclopropylmethyl)(4-methoxybenzoyl)amino]-1,3,4-thiadiazol-2-yl}benzyl)azetidine-3-carboxylic acid
1-(4-{5-[Benzoyl(butyl)amino]-1,3,4-thiadiazol-2-yl}benzyl)azetidine-3-carboxylic acid
1-(4-{5-[Butyl(2-fluorobenzoyl)amino]-1,3,4-thiadiazol-2-yl}-2-methylbenzyl)-azetidine-3-carboxylic acid
1-(4-{5-[Butyl(2-chlorobenzoyl)amino]-1,3,4-thiadiazol-2-yl}benzyl)azetidine-3-carboxylic acid
1-(4-{5-[Ethyl(phenylacetyl)amino]-1,3,4-thiadiazol-2-yl}benzyl)azetidine-3-carboxylic acid
1-(4-{5-[Ethyl(2-methoxybenzoyl)amino]-1,3,4-thiadiazol-2-yl}benzyl)azetidine-3-carboxylic acid
1-(4-{5-[(2-Chlorobenzoyl)(ethyl)amino]-1,3,4-thiadiazol-2-yl}benzyl)azetidine-3-carboxylic acid
1-(4-{5-[Butyl(2-fluorobenzoyl)amino]-1,3,4-thiadiazol-2-yl}-3-methylbenzyl)-pyrrolidine-3-carboxylic acid
1-(4-{5-[Benzoyl(ethyl)amino]-1,3,4-thiadiazol-2-yl}benzyl)azetidine-3-carboxylic acid
1-(4-{5-[Butyl(2-fluorobenzoyl)amino]-1,3,4-thiadiazol-2-yl}benzyl)pyrrolidine-3-carboxylic acid
1-(4-{5-[Butyl(phenylacetyl)amino]-1,3,4-thiadiazol-2-yl}-3-methylbenzyl)azetidine-3-carboxylic acid
1-[4-(5-{Butyl[(2-chlorophenyl)acetyl]amino}-1,3,4-thiadiazol-2-yl)-3-methylbenzyl]-azetidine-3-carboxylic acid
1-(4-{5-[Ethyl(3-fluorobenzoyl)amino]-1,3,4-thiadiazol-2-yl}benzyl)azetidine-3-carboxylic acid
1-[4-(5-{Butyl[(2-chlorophenyl)acetyl]amino}-1,3,4-thiadiazol-2-yl)-3-methylbenzyl]-pyrrolidine-3-carboxylic acid
1-(4-{5-[Butyl(2-chlorobenzoyl)amino]-1,3,4-thiadiazol-2-yl}-3-methylbenzyl)-azetidine-3-carboxylic acid
N-{5-[4-(2-Aminoethoxy)-3,5-dimethylphenyl]-1,3,4-thiadiazol-2-yl}-N-butyl-2-fluorobenzamide
1-(4-{5-[Butyl(2-fluorobenzoyl)amino]-1,3,4-thiadiazol-2-yl}-2,6-dimethylbenzyl)-azetidine-3-carboxylic acid
N-Butyl-N-{5-[4-(2,3-dihydroxypropoxy)-3,5-dimethylphenyl]-1,3,4-thiadiazol-2-yl}-2-methylbenzamide
1-(4-{5-[(3-Chloro-2-fluorobenzoyl)(ethyl)amino]-1,3,4-thiadiazol-2-yl}benzyl)-azetidine-3-carboxylic acid
1-(4-{5-[Butyl(3-chloro-2-fluorobenzoyl)amino]-1,3,4-thiadiazol-2-yl}-3-methylbenzyl)piperidine-4-carboxylic acid
1-(4-{5-[Butyl(3-chloro-2-fluorobenzoyl)amino]-1,3,4-thiadiazol-2-yl}-3-methyl benzyl)azetidine-3-carboxylic acid
N-Butyl-3-chloro-N-{5-[4-(2,3-dihydroxypropoxy)-3,5-dimethylphenyl]-1,3,4-thiadiazol-2-yl}-2-fluorobenzamide
1-(4-{5-[(3-Chloro-2-fluorobenzoyl)(ethyl)amino]-1,3,4-thiadiazol-2-yl}benzyl)-piperidine-4-carboxylic acid
N-{5-[4-(3-Amino-2-hydroxypropoxy)-3,5-dimethylphenyl]-1,3,4-thiadiazol-2-yl}-N-butyl-2-fluorobenzamide
1-(4-{5-[(3-Chloro-2-fluorobenzoyl)(cyclopropylmethyl)amino]-1,3,4-thiadiazol-2-yl}benzyl)piperidine-4-carboxylic acid
1-(4-{5-[Butyl(3-chloro-2-fluorobenzoyl)amino]-1,3,4-thiadiazol-2-yl}-2,6-dimethyl-benzyl)azetidine-3-carboxylic acid
1-(4-{5-[Butyl(3-chloro-2-fluorobenzoyl)amino]-1,3,4-thiadiazol-2-yl}-2,6-dimethylbenzyl)piperidine-4-carboxylic acid
1-(4-{5-[Butyl(2-chlorobenzoyl)amino]-1,3,4-thiadiazol-2-yl}-2,6-dimethyl-benzyl)azetidine-3-carboxylic acid
1-(4-{5-[Butyl(2-chlorobenzoyl)amino]-1,3,4-thiadiazol-2-yl}-3-methylbenzyl)-piperidine-4-carboxylic acid
1-(4-{5-[Butyl(2-chlorobenzoyl)amino]-1,3,4-thiadiazol-2-yl}-2,6-dimethylbenzyl)-piperidine-4-carboxylic acid
   Of outstanding interest are:
N-butyl-2-chloro-N-[5-(4-hydroxy-3,5-dimethylphenyl)-1,3,4-thiadiazol-2-yl]benzamide
N-butyl-2-fluoro-N-[5-(4-methoxy-3,5-dimethylphenyl)-1,3,4-thiadiazol-2-yl]benzamide
N-Butyl-2-fluoro-N-[5-(4-hydroxy-3,5-dimethylphenyl)-1,3,4-thiadiazol-2-yl]benzamide
N-butyl-2-methoxy-N-[5-(4-methoxy-3,5-dimethylphenyl)-1,3,4-thiadiazol-2-yl]benzamide
N-butyl-2-chloro-N-{5-[4-(2-methoxyethoxy)-3,5-dimethylphenyl]-1,3,4-thiadiazol-2-yl}benzamide
2-Chloro-N-[5-(4-methoxy-3,5-dimethylphenyl)-1,3,4-thiadiazol-2-yl]-N-(2-methoxyethyl)benzamide
N-Butyl-N-[5-(2-chloro-6-methoxypyridin-4-yl)-1,3,4-thiadiazol-2-yl]-2-fluorobenzamide
2-fluoro-N-[5-(4-methoxy-3,5-dimethylphenyl)-1,3,4-thiadiazol-2-yl]-N-propylbenzamide
N-Butyl-2-fluoro-N-[5-(2-methylpyridin-4-yl)-1,3,4-thiadiazol-2-yl]benzamide
N-(cyclopropylmethyl)-2-fluoro-N-[5-(4-methoxy-3,5-dimethylphenyl)-1,3,4-thiadiazol-2-yl]benzamide
3-(4-{5-[butyl(2-fluorobenzoyl)amino]-1,3,4-thiadiazol-2-yl}-2,6-dimethyl-phenyl)propanoic acid
N-Butyl-2-chloro-N-[5-(2-chloro-6-methylpyridin-4-yl)-1,3,4-thiadiazol-2-yl]benzamide
3-(4-(5-(N-butyl-2-chlorobenzamido)-1,3,4-thiadiazol-2-yl)benzamido)propanoic acid
2-fluoro-N-(5-(4-methoxy-3,5-dimethylphenyl)-1,3,4-thiadiazol-2-yl)-N-phenethylbenzamide
2-(4-(5-(N-butyl-2-fluorobenzamido)-1,3,4-thiadiazol-2-yl)benzamido)acetic acid
N-(4-{5-[butyl(2-fluorobenzoyl)amino]-1,3,4-thiadiazol-2-yl}benzyl)-beta-alanine
1-(4-{5-[(2-fluorobenzoyl)(propyl)amino]-1,3,4-thiadiazol-2-yl}benzyl)azetidine-3-carboxylic acid
1-(4-{5-[(cyclopropylmethyl)(2-fluorobenzoyl)amino]-1,3,4-thiadiazol-2-yl}benzyl)-azetidine-3-carboxylic acid
1-(4-{5-[butyl(2-fluorobenzoyl)amino]-1,3,4-thiadiazol-2-yl}-3-methylbenzyl) azetidine-3-carboxylic acid
1-(4-{5-[butyl(phenylacetyl)amino]-1,3,4-thiadiazol-2-yl}benzyl)azetidine-3-carboxylic acid
1-[4-(5-{butyl[(2-fluorophenyl)acetyl]amino}-1,3,4-thiadiazol-2-yl)benzyl]azetidine-3-carboxylic acid
1-(4-{5-[butyl(2-fluorobenzoyl)amino]-1,3,4-thiadiazol-2-yl}-2-methylbenzyl)-azetidine-3-carboxylic acid
1-(4-{5-[butyl(2-fluorobenzoyl)amino]-1,3,4-thiadiazol-2-yl}-3-methylbenzyl)-pyrrolidine-3-carboxylic acid
1-(4-{5-[butyl(2-chlorobenzoyl)amino]-1,3,4-thiadiazol-2-yl}-3-methylbenzyl)azetidine-3-carboxylic acid
1-(4-{5-[butyl(2-fluorobenzoyl)amino]-1,3,4-thiadiazol-2-yl}-2,6-dimethylbenzyl)azetidine-3-carboxylic acid
1-(4-{5-[(3-chloro-2-fluorobenzoyl)(ethyl)amino]-1,3,4-thiadiazol-2-yl}benzyl)-azetidine-3-carboxylic acid
1-(4-{5-[butyl(3-chloro-2-fluorobenzoyl)amino]-1,3,4-thiadiazol-2-yl}-3-methyl-benzyl)piperidine-4-carboxylic acid
1-(4-{5-[butyl(3-chloro-2-fluorobenzoyl)amino]-1,3,4-thiadiazol-2-yl}-3-methyl-benzyl)azetidine-3-carboxylic acid
N-{5-[4-(3-amino-2-hydroxypropoxy)-3,5-dimethylphenyl]-1,3,4-thiadiazol-2-yl}-N-butyl-2-fluorobenzamide
1-(4-{5-[butyl(3-chloro-2-fluorobenzoyl)amino]-1,3,4-thiadiazol-2-yl}-2,6-dimethyl-benzyl)azetidine-3-carboxylic acid
1-(4-[(5-[butyl(3-chloro-2-fluorobenzoyl)amino]-1,3,4-thiadiazol-2-yl]-2,6-dimethyl-benzyl)piperidine-4-carboxylic acid
1-(4-{5-[butyl(2-chlorobenzoyl)amino]-1,3,4-thiadiazol-2-yl}-2,6-dimethylbenzyl)azetidine-3-carboxylic acid, and
1-(4-{5-[butyl(2-chlorobenzoyl)amino]-1,3,4-thiadiazol-2-yl}-2,6-dimethylbenzyl)piperidine-4-carboxylic acid

Compounds of general formula (I) may be prepared following the synthetic scheme depicted in FIG. 1.

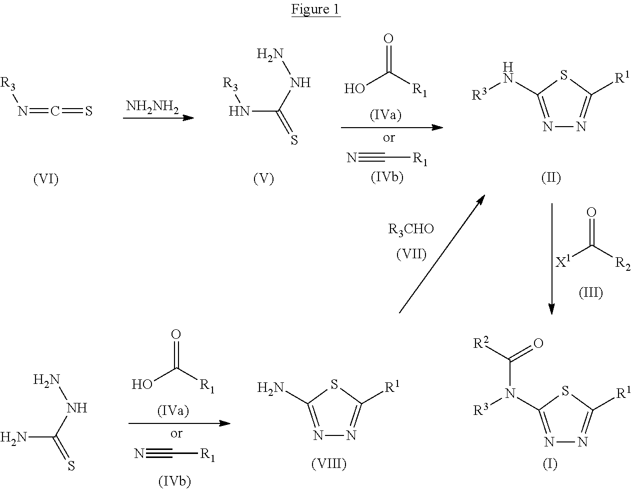

Compounds of general formula (I) may be prepared by the reaction of 1,3,4-thiadiazol-2-amine derivatives (II), wherein R¹ and R³ are as described above, with the corresponding acylating agent (III), wherein R² is as described above and X¹ represents a hydroxy group or a chlorine atom. When X¹ is a chlorine atom, the reaction takes place in basic media such as triethylamine, pyridine or diisopropylethylamine with a solvent such as dichloromethane, DMF, THF or pyridine at a temperature between 20 and 150° C. and in a standard reactor or in a microwave apparatus. These reactions may be catalyzed by 4-dimethylaminopyridine. When X¹ is a hydroxy group, a coupling agent is used such as 2-(1H-7-Azabenzotriazol-1-yl)-1,1,3,3-tetramethyl uronium hexafluorophosphate (HATU) or O-Benzotriazole-N,N,N′,N′-tetramethyl-uronium-hexafluoro-phosphate HBTU, with a base such as triethylamine, pyridine or diisopropylethylamine, in the presence of a solvent such as dichloromethane, DMF, THF or pyridine, at a temperature between 20 and 150° C. and in a standard reactor or in a microwave apparatus. Other coupling agents such as 1-[3-(dimethylamino)propyl]-3-ethylcarbodiimide hydrochloride EDC or dicyclohexylcarbodiimide DCC, may be used in the presence of N-hydroxybenzotriazole HOBt as a catalyst in a solvent such as dichloromethane, DMF or THF at a temperature between 20 and 150° C. and in a standard reactor or in a microwave apparatus.

Compounds of general formula (II) may be prepared by condensation of the compounds of formula (V) with the corresponding acid derivative (IVa) using POCl₃. Alternatively they may be prepared by condensation of compounds of formula (V) with the corresponding nitrile (IVb) in the presence of trifluoroacetic acid. Both reactions may be performed without a solvent or in a solvent such as dioxane or THF or dichloromethane at a temperature from 20 to 150° C.

Intermediates of formula (V) may be obtained by the reaction of hydrazine hydrate with the corresponding isothiocyanate (VI) in a solvent such as THF, methanol or ethanol and at a temperature from 0 to 40° C.

Alternatively, Intermediates of general formula (II) may be prepared by reductive amination of compounds of general formula (VIII) with the corresponding aldehyde (VII) in acid media such as acetic acid and in a protic solvent such as methanol or ethanol and with a reductive agent such as sodium borohydride or sodium cyanoborohydride at a temperature from 0° C. to the boiling point of the solvent.

Intermediates of general formula (VIII) may be prepared by condensation of hydrazinecarbothioamide with the corresponding acid derivative (IVa) using POCl₃ or with the corresponding nitrile derivative (IVb) in the presence of TFA. Both reactions may be performed without a solvent in a solvent such as dioxane or THF or dichloromethane at a temperature from 20 to 100° C.

In the particular case where R¹ represents a group of formula

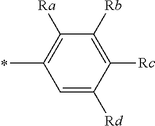

and Rc is a C₁ alkoxy group of formula —OG, wherein G is the alkyl radical of the alkoxy group which is substituted with one or more substituents, the compounds of formula (Ia) may be obtained following the synthetic path shown in FIG. 2.

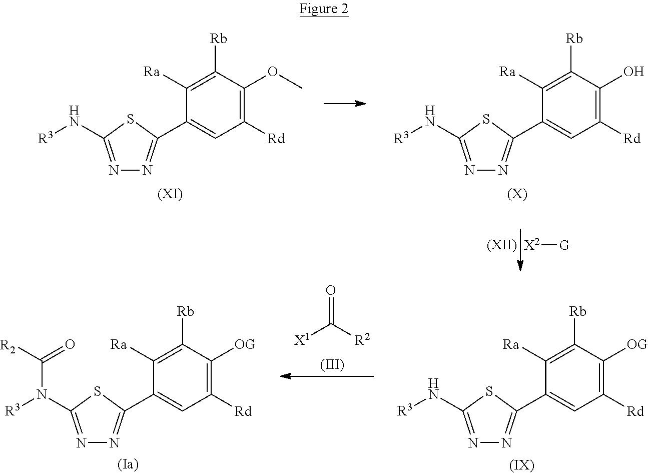

The compounds of formula (Ia) may be obtained by the reaction of 1,3,4-thiadiazol-2-amine derivatives (IX), wherein Ra, Rb, Rd, R³ and G are as described above, with the corresponding acylating agent (III) wherein R² and X¹ are as described above, following the same procedure used for the preparation of compounds of formula (I). 1,3,4-Thiadiazol-2-amine derivatives of formula (IX) may be obtained by the reaction of the phenol derivatives of formula (X) with the corresponding alkylating agent (XII), wherein X² is an halogen atom such as chlorine, bromine or iodide or X² is a sulphonate derivative such as mesylate, tosylate or triflate in basic media such as sodium hydride in a solvent such as THF or DMF at a temperature from 0 to 150° C. Alternatively, the phenolic functionality of (X) may be coupled to suitable alcohol derivatives HO-G, wherein G is as defined above, using a Mitsunobu coupling procedure (Mitsunobu, O., Synthesis 1 (1981)). Preferred coupling conditions include the use of a trialkylphosphine or triarylphosphine, such as tri-n-butylphosphine or triphenylphosphine, in a suitable solvent, such as tetrahydrofuran or dichloromethane, and an azodicarbonyl reagent, such as diethyl azodicarboxylate or 1,1′-(azodicarbonyl)dipiperidine.

The compounds of general formula (X) may be prepared by demethylation of the corresponding compound of general formula (XI) using BBr₃ or AlBr₃ or BF₃ as demethylating agent in a solvent such as dichloromethane or 1,2-dichloroethane, chloroform at a temperature between 0 and the 60° C. Alternatively compounds of general formula (X) may be prepared by demethylation using HBr in acetic acid as a solvent.

Finally, compounds of formula (XI) may be obtained from the condensation of the compounds of general formula (V) with the corresponding acid derivative (IVa) or nitrile derivative (IVb) or by the reductive amination of compounds of general formula (VIII) with the corresponding aldehyde of general formula (VII), as described for the compounds of formula (II) depicted in FIG. 1.

In the particular case where R¹ represents a group of formula

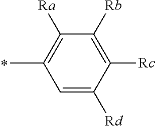

and Rc is a C₁₋₄ alkoxy group of formula —OG, wherein G is a 2,3-dihydroxypropyl, the compounds of formula (Iab) may also be obtained following the synthetic path shown in FIG. 2b.

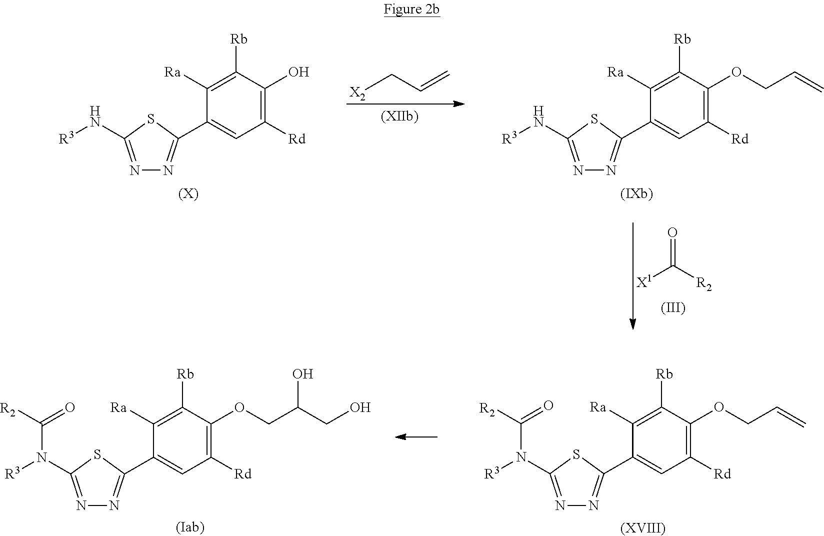

The compounds of formula (Iab) may be obtained by dihydroxylation of 1,3,4-thiadiazol-2-amide derivatives (XVIII), wherein Ra, Rb, Rd, R² and R³ are as described above, using a catalytic amount of an oxidizing agent such as osmium tetroxide and a cooxidant such as N-methylpyrrolidone N-oxide in a mixture of solvents such as tetrahydrofurane, tert-butanol, methanol, ethanol, acetonitrile or water at a temperature from 0° C. to the boiling point of the mixture. Compounds of general formula (XVIII) can be obtained by acylation of compounds of general formula (IXb) with compounds of general formula (III) by methods described above. Finally, compounds of general formula (IXb) can be obtained by the reaction of the phenol derivatives of formula (X) with the corresponding alkylating agent (XIIb), wherein X² is an halogen atom such as chlorine, bromine or iodide or a sulphonate derivative such as mesylate, tosylate or triflate in basic media such as sodium hydride in a solvent such as THF or DMF at a temperature from 0 to 150° C.

In the particular case where R¹ represents a group of formula,

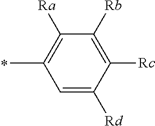

Rc is —(CH₂)₍₀₋₄₎-L-R⁵ and L is a group of formula

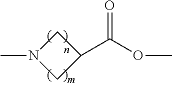

wherein n and m are as described above, the compound of formula (Ib) may be obtained following the synthetic path shown in FIG. 3.

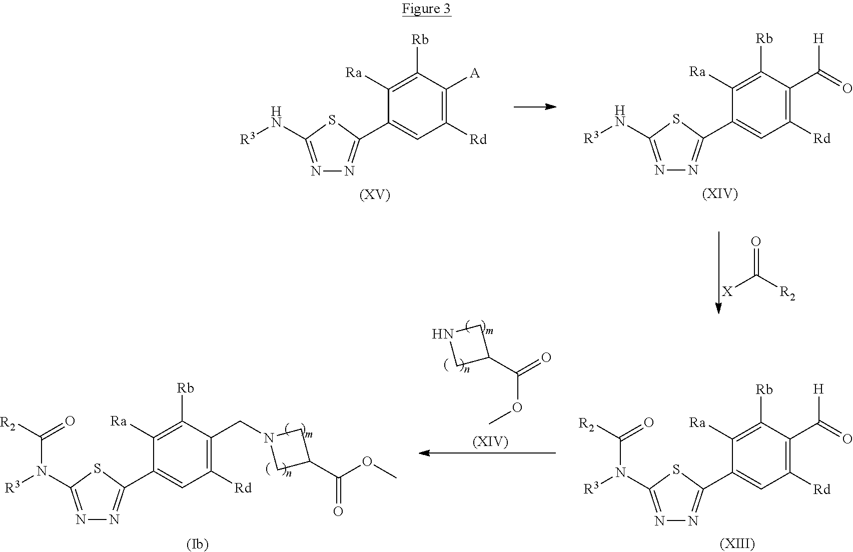

Compounds of general formula (Ib), wherein Ra, Rb, Rd, R², R³, n and m are as described above may be prepared by the reductive amination of the aldehyde derivatives of general formula (XIII) with the corresponding aminoacid of formula (XIV) in acid media such as acetic acid and in a protic solvent such as methanol or ethanol and with a reductive agent such as sodium borohydride or sodium cyanoborohydride at a temperature from 0° C. to the boiling point of the solvent. As an alternative to the acidic media a Lewis acid such as zinc chloride can be used.

Compounds of formula (XIII) may be obtained by acylation of compounds of general formula (XIV) with the corresponding acylating agent of formula (III) by standard methods as described before.

Finally, aldehyde derivatives of general formula (XIV) may be obtained by reduction of the corresponding nitrile of general formula (XV), wherein A represents CN, by using a reductive agent such as DIBAL-H in a solvent such as dichloromethane, THF or dioxane at a low temperature from −78° to 0° C.

Alternatively, compounds of general formula (XIV) may also be obtained by reduction of the corresponding ester (XV), wherein A represents a —COOR radical, to the alcohol using LiAlH₄ as reductive agent in a solvent such as THF and the following oxidation to the corresponding aldehyde of formula (XIV) by a standard Swern oxidation or other oxidizing agents such as Dess-Martin reagent.

An alternative way for preparing intermediates of formula (XIII) is as depicted in FIG. 3b)

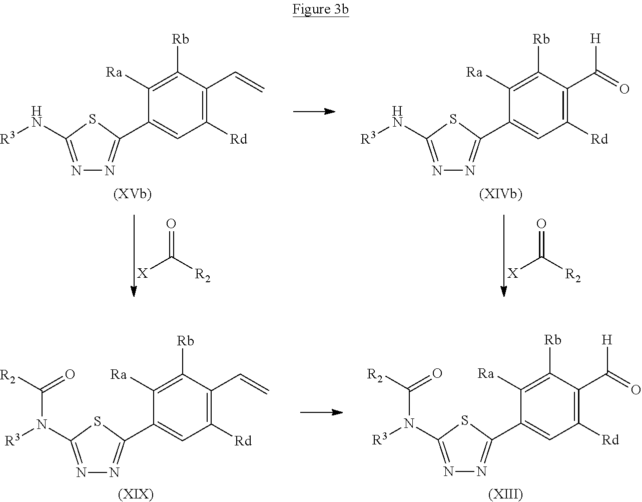

Intermediates of general formula (XIII) may be obtained by dihydroxylation and oxidative cleavage of the corresponding vinyl derivative (XIX), using a catalytic amount of an oxidizing agent such as osmium tetroxide and a cooxidant such as sodium periodate in a mixture of solvents such as methanol, acetonitrile and water at a temperature from 0° C. to the boiling point of the mixture. Compounds of general formula (XIX) can be obtained by acylation of compounds of general formula (XVb) by methods described above.

Alternatively, intermediates of general formula (XIII) may also be obtained by acylation of compounds of general formula (XIVb) by methods described above. Compounds of general formula (XIVb) may be obtained form compounds of general formula (XVb) by dihydroxylation and oxidative cleavage, using a catalytic amount of an oxidizing agent such as osmium tetroxide and a cooxidant such as sodium periodate in a mixture of solvents such as methanol, acetonitrile and water at a temperature from 0° C. to the boiling point of the mixture.

In the particular case where R¹ represents a group of formula

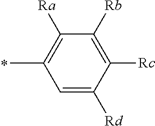

and Rc is CH₂—NH—(CH₂)₍₀₋₄₎—COOH, the compounds of formula (Ic) may also be obtained by the reductive amination of the aldehyde derivatives of general formula (XIII) with the corresponding aminoacid of formula (XVI) in acid media such as acetic acid and in a protic solvent such as methanol or ethanol and with a reductive agent such as sodium borohydride or sodium cyanoborohydride at a temperature from 0° C. to the boiling point of the solvent as shown in FIG. 4.

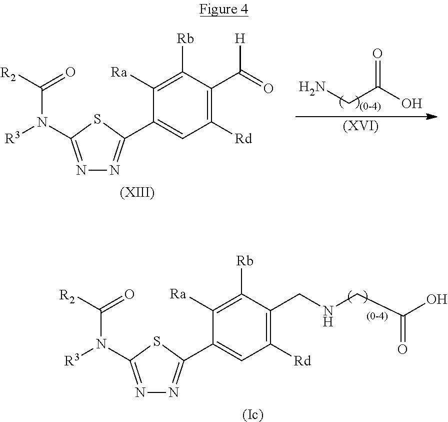

The compounds of general formula (Ib) may be prepared by the reductive amination of the aldehyde derivatives of general formula (XIII) with the corresponding aminoacid of formula (XVI) in acid media such as acetic acid and in a protic solvent such as methanol or ethanol and with a reductive agent such as sodium borohydride or sodium cyanoborohydride at a temperature from 0° C. to the boiling point of the solvent. As an alternative to the acidic media a Lewis acid such as zinc chloride can be used.

In the particular case where R¹ represents a group of formula

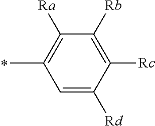

and Rc is —C(O)NH—R⁵ the compounds of formula (Id) may be obtained following the synthetic path shown in FIG. 5.

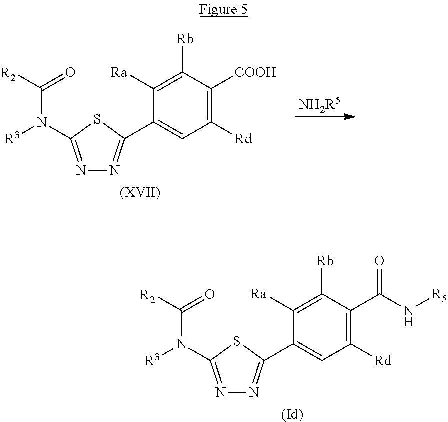

Reaction of the acid derivatives of general formula (XVII) with the corresponding amine using as a coupling agent HATU or HBTU with a base such as triethylamine, pyridine or diisopropylethylamine, with a solvent such as dichloromethane, DMF, THF or pyridine, or using as a coupling agent EDC or DCC, with HOBt and in a solvent such as dichloromethane, DMF or THF, yield the final compounds of formula (Id).

In the particular case where R¹ represents a group of formula,

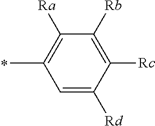

Rc is —(CH₂)₂-L-R⁵ and L is a group of formula

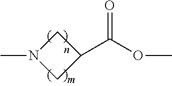

wherein n and m are as described above, the compound of formula (Ie) may be obtained following the synthetic path shown in FIG. 6.

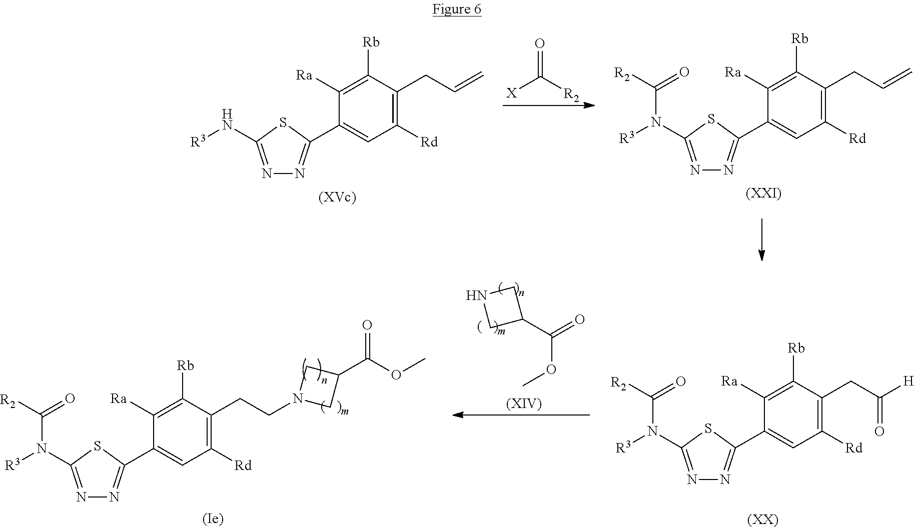

Compounds of general formula (Ie), wherein Ra, Rb, Rd, R², R³, n and m are as described above may be prepared by the reductive amination of the aldehyde derivatives of general formula (XX) with the corresponding aminoacid of formula (XIV) in acid media such as acetic acid and in a protic solvent such as methanol or ethanol and with a reductive agent such as sodium borohydride or sodium cyanoborohydride at a temperature from 0° C. to the boiling point of the solvent. As an alternative to the acidic media a Lewis acid such as zinc chloride can be used.

Compounds of general formula (XX) may be obtained by dihydroxylation and oxidative cleavage of the corresponding allyl derivative (XXI), using a catalytic amount of an oxidizing agent such as osmium tetroxide and a cooxidant such as sodium periodate in a mixture of solvents such as methanol, acetonitrile and water at a temperature from 0° C. to the boiling point of the mixture. Finally, compounds of general formula (XXI) can be obtained by acylation of compounds of general formula (XVc) by methods described above.

When the defined groups R¹ to R⁵ and Ra to Rd are susceptible to chemical reaction under the conditions of the hereinbefore described processes or are incompatible with said processes, conventional protecting groups may be used in accordance with standard practice, for example see T. W. Greene and P. G. M. Wuts in “protective Groups in Organic Chemistry”, 3rd Edition, John Wiley&Sons (1999). It may be that deprotection will form the last step in the synthesis of compounds of formula (I).

The syntheses of the compounds of the invention and their intermediates are illustrated by the following Examples (1 to 119) including Preparation Examples (1 to 169) which do not limit the scope of the invention in any way.

Starting compounds are commercially available or may be obtained following the conventional synthetic method already known in the art.

¹H Nuclear Magnetic Resonance Spectra were recorded on a Varian Mercury 200 spectrometer. Low Resolution Mass Spectra (m/z) were recorded on a Micromass ZMD mass spectrometer using ESI ionization. The chromatographic separations were obtained using a Waters 2690 system equipped with a Symmetry C18 (2.1×50 mm, 3.5 μM) column for method A and B and a Symmetry C18 (2.1×100 mm, 3.5 μM) for method C. The mobile phase was formic acid (0.4 mL), ammonia (0.1 mL), methanol (500 mL) and acetonitrile (500 mL) (B) and formic acid (0.46 mL), ammonia (0.115 mL) and water (1000 mL) (A), the gradients are specified in the following table for each method used. The reequilibration time between two injections was 1 min. The flow rate was 0.8 mL/min for method A and 0.4 mL/min for method B and C. The injection volume was 5 microliter for method A and B and 3 microliter for method C. Diode array chromatograms were collected at 210 nM.

| Method | 0% B | 0 to 95% B | 95% B |
|---|---|---|---|
| A | 0.2 min | 3 min | 0.8 min |
| B | 0.5 min | 6.5 min | 1 min |
| C | 0 min | 20 min | 4 min |

### Purification Method A:

The solid was dissolved in DMSO/MeOH, injected into a Biotage C18 silica column (40M, 25M or 25S according to the crude amount) and eluted on the SP1® automated purification system from Biotage. The gradient used was H2O/Acetonitrile/MeOH (1:1) (0.1% v/v HCOONH₄ both phases) from 0% to 100% acetonitrile/MeOH (1:1) in 80 column volumes. The appropriate fractions were collected and the organic solvent evaporated under reduced pressure or lyophilized.

### PREPARATION EXAMPLES

### Preparation 1

### N-Butylhydrazinecarbothioamide

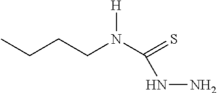

To a 10° C. stirred solution of hydrazine hydrate (32 ml, 0.66 mol) in methanol (500 ml), butylisothiocyanate (25 g, 0.22 mol) was added dropwise keeping the temperature between 10-15° C. The final mixture was stirred for 1 h at 10° C. and the solvent was removed to yield an oil that was lyophilized. A white solid was obtained. It was washed with diethyl ether. 28 g (87% yield).

LRMS: m/z 148 (M+1)⁺

Retention time: 4.17 min (method B)

¹H NMR (200 MHz, CDCl₃) δ ppm 1.0 (t, J=7.0 Hz, 3H) 1.4 (m, 2H) 1.6 (m, 2H) 3.6 (m, 2H) 3.8 (s, 2H) 7.4 (s, 1H) 7.8 (s, 1H)

### Preparation 2

### 4-(5-(Butylamino)-1,3,4-thiadiazol-2-yl)benzenesulfonamide

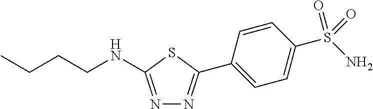

To a stirred suspension of 4-sulfamoylbenzoic acid (1.36 g, 6.8 mmol) and N-butylhydrazinecarbothioamide (Preparation 1) (1 g, 6.8 mmol) in dioxane (8 ml), POCl3 (1.46 ml, 15.7 mmol) was added dropwise and the final mixture was stirred at 70° C. for 3 h. The mixture was let to cool down and was carefully poured onto ice water. The solid thus obtained was filtered and suspended in water. It was basified to pH 12 and the solution thus formed was neutralized. The solid thus formed was filtered and thoroughly washed with water and hexanes to yield 1.26 g (63%) of the title compound.

LRMS: m/z 313 (M+1)⁺

Retention time: 10.31 min (method C)

¹H NMR (200 MHz, DMSO-d₆) δ ppm 0.92 (t, J=7.22 Hz, 3H) 1.11-1.79 (m, 4H) 3.40 (m, 2H) 7.47 (s, 2H) 7.68-8.03 (m, 4H) 8.12 (t, J=5.27 Hz, 1H)

### Preparation 3

### Tert-butyl ({4-[5-(butylamino)-1,3,4-thiadiazol-2-yl]phenyl}sulfonyl)carbamate

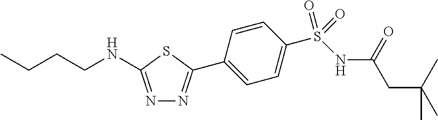

To a stirred suspension of the title compound of Preparation 2 (1 g, 3.20 mmol) in dichloromethane (30 ml), ethyldiisopropylamine (0.64 ml, 3.69 mmol) and 4-DMAP (391 mg, 3.2 mmol) were added and the mixture was stirred for a while. Then Boc₂O (803 mg, 3.69 mmol) was added and the final mixture was stirred at rt overnight. The reaction crude was diluted with dichloromethane and washed with water and HCl 2 M. The organic layer was dried and solvent was removed to yield 1.32 g of a 1:1 mixture of the title compound and 4-DMAP which was used without further purification. Yield 75%

LRMS: m/z 413 (M+1)⁺

Retention time: 3.21 min (method A)

¹H NMR (200 MHz, DMSO-d₆) δ ppm 0.91 (t, J=7.22 Hz, 3H) 1.15 (s, 9H) 1.35 (m, 2H) 1.60 (m, 2H) 3.52 (q, 2H) 7.75 (s, 4H) 8.10 (t, 1H)

### Preparation 4

### tert-Butyl 4-(5-(N-butyl-3-methoxybenzamido)-1,3,4-thiadiazol-2-yl)phenyl-sulfonylcarbamate

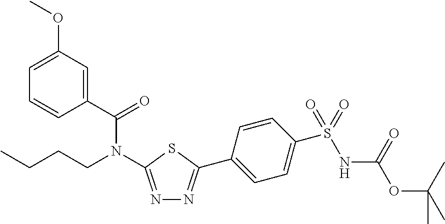

To a stirred solution of the title compound of Preparation 3 (88 mg, 0.52 mmol) in dichloromethane (5 ml), ethyldiisopropylamine (0.360 ml, 2.07 mmol) and 3-methoxybenzoyl chloride were added and the mixture was stirred at rt overnight. Solvent was removed and the solid thus obtained was purified according to purification method A. 120 mg (44% yield) were obtained.

LRMS: m/z 547 (M+1)⁺

Retention time: 17.11 min (method C)

¹H NMR (200 MHz, DMSO-d₆) δ ppm 0.91 (t, 3H) 1.18 (q, 2H) 1.35 (s, 9H) 1.60 (m, 2H) 3.81 (s, 3H) 4.15 (t, 2H) 7.25 (m, 3H) 7.45 (m, 1H) 8.02 (d, 2H) 8.25 (d, 2H)

### Preparation 5

### tert-Butyl 4-(5-(N-butyl-2-naphthamido)-1,3,4-thiadiazol-2-yl)phenylsulfonyl-carbamate

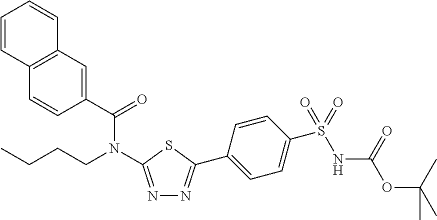

Obtained (60% yield) from the title compound of Preparation 3 and 2-naphtoyl chloride following the experimental procedure of Preparation 4.

LRMS: m/z 567 (M+1)⁺

Retention time: 3.85 min (method A)

¹H NMR (200 MHz, DMSO-D6) δ ppm 0.7 (t, J=7.2 Hz, 3H) 1.1 (m, 2H) 1.3 (s, 9H) 1.7 (m, 2H) 4.2 (m, 2H) 7.7 (m, 3H) 8.1 (m, 6H) 8.3 (m, 3H)

### Preparation 6

### tert-Butyl 4-(5-(N-butylbiphenyl-4-ylcarboxamido)-1,3,4-thiadiazol-2-yl)phenyl-sulfonylcarbamate

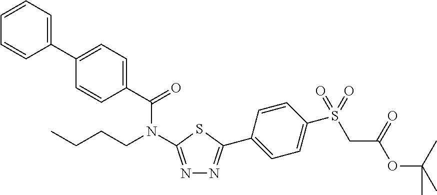

Obtained (49% yield) from the title compound of Preparation 3 and biphenyl-4-carbonyl chloride following the experimental procedure of Preparation 4.

LRMS: m/z 593(M+1)⁺

Retention time: 3.89 min (method A)

¹H NMR (200 MHz, DMSO-D6) δ ppm 0.7 (t, J=7.4 Hz, 3H) 1.2 (m, 2H) 1.3 (s, 9H) 1.7 (m, 2H) 4.2 (m, 2H) 7.5 (m, 3H) 7.8 (d, J=6.6 Hz, 4H) 7.9 (m, 4H) 8.1 (d, J=9.8 Hz, 2H) 8.1 (s, 1H)

### Preparation 7

### tert-Butyl 4-(5-(4-butoxy-N-butylbenzamido)-1,3,4-thiadiazol-2-yl)phenylsulfonyl-carbamate

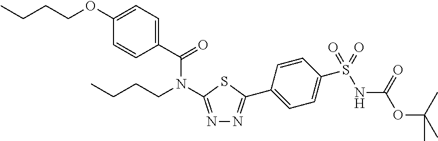

Obtained (49% yield) from the title compound of Preparation 3 and 4-butoxybenzoyl chloride following the experimental procedure of Preparation 4.

LRMS: m/z 589(M+1)⁺

Retention time: 3.95 min (method A)

¹H NMR (200 MHz, DMSO-D6) δ ppm 0.7 (t, J=7.2 Hz, 3H) 0.9 (t, J=7.2 Hz, 3H) 1.1 (m, 2H) 1.3 (s, 9H) 1.5 (m, 2H) 1.7 (m, 4H) 4.1 (t, J=6.4 Hz, 2H) 4.2 (m, 2H) 7.1 (d, J=9.0 Hz, 2H) 7.6 (d, J=8.6 Hz, 2H) 7.9 (d, J=7.4 Hz, 2H) 8.1 (m, 3H)

### Preparation 8

### tert-Butyl 4-(5-(N-butylbenzamido)-1,3,4-thiadiazol-2-yl)phenylsulfonylcarbamate

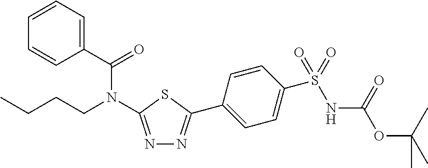

Obtained (20% yield) from the title compound of Preparation 3 and benzoyl chloride following the experimental procedure of Preparation 4.

LRMS: m/z 517(M+1)⁺

Retention time: 3.71 min (method A)

### Preparation 9

### N-Butyl-5-(4-methoxy-3,5-dimethylphenyl)-1,3,4-thiadiazol-2-amine

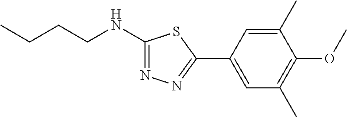

Obtained (99% yield) from the title compound of Preparation 1 and 4-methoxy-3,5-dimethylbenzoic acid following the experimental procedure of Preparation 2. The final product was recrystallized from isopropanol.

LRMS: m/z 292(M+1)⁺

Retention time: 6.67 min (method B)

¹H NMR (200 MHz, DMSO-d₆) δ ppm 0.92 (t, J=7.22 Hz, 3H) 1.39 (dq, J=14.54, 7.13 Hz, 2H) 2.28 (m, 2H) 3.38 (t, J=6.64 Hz, 2H) 3.70 (s, 3H) 4.58-5.60 (m, 6H) 7.48 (s, 2H).

### Preparation 10

### 5-(4-Bromophenyl)-N-butyl-1,3,4-thiadiazol-2-amine

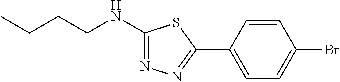

To a stirred solution of 5-(4-bromophenyl)-1,3,4-thiadiazol-2-amine (2.0 g, 7.81 mmol) in methanol (60 ml), butyraldehyde (2.13 g, 29 mmol) and AcOH (two drops) were added and the mixture was stirred at 60° C. for 48 h. Then it was cooled to 0° C. and NaBH4 (1.12 g, 29 mmol) was added portionwise and the final mixture was stirred at rt for 2 h. Solvent was removed and the solid thus obtained was partitioned between dichloromethane and water. The organic layer was washed with water and brine. It was dried and solvent removed to yield a crude product that was purified on a Biotage 40S chromatography column of silica gel using mixtures of hexane/ethyl acetate as eluent. 2.44 g (25% yield).

LRMS: m/z 312(M+1)⁺

Retention time: 6.77 min (method B)

¹H NMR (200 MHz, DMSO-d₆) δ ppm 0.91 (t, J=7.03 Hz, 3H) 1.09-1.81 (m, 4H) 7.83 (d, 2H) 8.01 (m, 3H)

### Preparation 11

### N-(5-(4-bromophenyl)-1,3,4-thiadiazol-2-yl)-N-butyl-3-methoxybenzamide

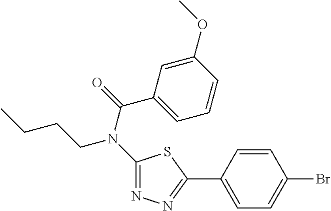

To a stirred suspension of the title compound of Preparation 10 (1.0 g, 3.20 mmol) and diisopropylethylamine (2.23 ml, 12.8 mmol) in dioxane (20 ml), 3-methoxybenzoyl chloride (1.10 g, 6.45 mmol) was added and the mixture was stirred at 80° C. for 4 h and then at rt overnight. Solvent was removed and the mixture was partitioned between water and ethyl acetate. The organic layer was washed with HCl 2N, with potassium carbonate and brine. Solvent was removed and the crude thus obtained was purified by column cromathography using a 40S Biotage column and a gradient from 100% hexane to hexane/ethyl ether 80:20. 450 mg (31% yield) of the title product were obtained.

LRMS: m/z 446(M+1)⁺

Retention time: 7.66 min (method B)

¹H NMR (200 MHz, DMSO-d₆) δ ppm 0.73 (t, J=7.22 Hz, 3H) 0.95-1.31 (m, 2H) 1.70 (m, 2H) 3.82 (s, 3H) 4.10 (t, J=7.22 Hz, 2H) 7.20 (d, J=8.59 Hz, 2H) 7.49 (d, J=7.61 Hz, 2H) 7.64-7.85 (m, 2H) 7.95 (d, J=8.20 Hz, 2H)

### Preparation 12

### Tert-butyl (2E)-3-{4-[5-(butylamino)-1,3,4-thiadiazol-2-yl]phenyl}acrylate

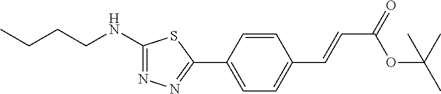

Under nitrogen atmosphere, a mixture of the title compound of Preparation 11 (300 mg, 0.67 mmol), tert-butyl acrylate (129 mg, 1.06 mmol), potassium carbonate (186 mg, 1.35 mmol) N,N-dimethylalanine (6.3 mg, 0.54 mmol) and palladium (II) acetate (1.5 mg, 0.006 mmol) in NMP (3 ml) was stirred at 120° C. overnight. It was let to cool down and the poured onto water and extracted with ethyl acetate. The organic layer was washed with water. Solvent was removed to yield 200 mg (80%) of the title compound.

LRMS: m/z 360 (M+1)⁺

Retention time: 7.30 min (method B)

### Preparation 13

### Tert-butyl 3-(4-(5-(butylamino)-1,3,4-thiadiazol-2-yl)phenyl)propanoate

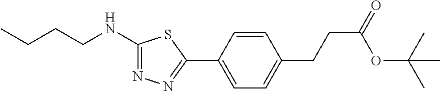

A mixture of the title compound of Preparation 12 (200 mg, 0.56 mmol) and Pd/C with a 50% of water (20 mg) in methanol (200 ml) was stirred under hydrogen at 20 psi overnight. 20 mg more of the palladium catalyst were added and the mixture was stirred under hydrogen at the same pressure for 3 days. The catalyst was filtered off and solvent was removed to yield a crude product that was purified following purification method A. 85 mg of the title compound (42%) were obtained.

LRMS: m/z 362 (M+1)⁺

Retention time: 7.06 min (method B)

¹H NMR (200 MHz, DMSO-D6) δ ppm 0.9 (t, J=7.2 Hz, 3H) 1.3 (m, 2H) 1.4 (s, 9H) 1.6 (m, 2H) 2.6 (m, 2H) 2.8 (t, J=7.2 Hz, 2H) 3.2 (m, 2H) 7.3 (d, J=7.8 Hz, 2H) 7.7 (d, J=7.8 Hz, 2H) 7.9 (t, J=5.5 Hz, 1H)

### Preparation 14

### tert-Butyl 3-(4-(5-(N-butyl-3-methoxybenzamido)-1,3,4-thiadiazol-2-yl)phenyl)propanoate

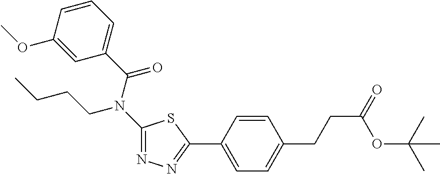

Obtained (63%) from the title compound of Preparation 13 and 3-methoxybenzoyl chloride following the experimental procedure of Preparation 11.

LRMS: m/z 496 (M+1)⁺

Retention time: 3.89 min (method A)

### Preparation 15

### 5-(4-Methoxy-3,5-dimethylphenyl)-N-methyl-1,3,4-thiadiazol-2-amine

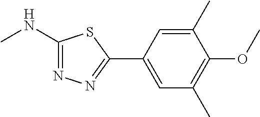

Obtained (65%) from 4-methoxy-3,5-dimethylbenzoic acid and N-methylhydrazinecarbothioamide following the experimental procedure of Preparation 2.

LRMS: m/z 250 (M+1)⁺

Retention time: 5.73 min (method B)

¹H NMR (200 MHz, DMSO-D6) δ ppm 2.3 (s, 6H) 2.9 (s, 3H) 3.7 (s, 3H) 7.4 (s, 2H) 7.8 (s, 1H)

### Preparation 16

### N-Butyl-5-(3,4-dimethoxyphenyl)-1,3,4-thiadiazol-2-amine

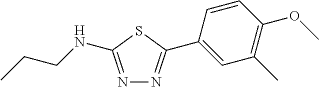

Obtained (89%) from 4-methoxy-3-methylbenzoic acid and N-butylhydrazinecarbothioamide following the experimental procedure of Preparation 2.

LRMS: m/z 278 (M+1)⁺

Retention time: 3.48 min (method A)

### Preparation 17

### N-Butyl-5-(3-chloro-4-methoxyphenyl)-1,3,4-thiadiazol-2-amine

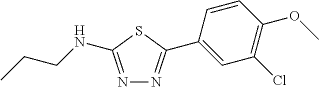

Obtained (68%) from 3-chloro-5-methoxybenzoic acid and N-butylhydrazinecarbothioamide following the experimental procedure of Preparation 2.

LRMS: m/z 298 (M+1)⁺

Retention time: 3.44 min (method A)

¹H NMR (200 MHz, DMSO-D6) δ ppm 0.9 (t, J=7.2 Hz, 3H) 1.4 (m, 2H) 1.6 (m, 2H) 3.3 (m, 2H) 3.9 (s, 3H) 7.2 (d, J=9.0 Hz, 1H) 7.7 (dd, J=8.6, 2.3 Hz, 1H) 7.8 (d, J=2.3 Hz, 1H) 7.9 (t, J=5.5 Hz, 1H)

### Preparation 18

### 4-(5-(Butylamino)-1,3,4-thiadiazol-2-yl)benzoic acid methyl esther

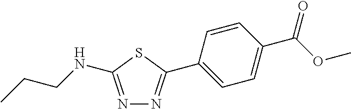

Obtained (38%) from 4-(methoxycarbonyl)benzoic acid and N-butylhydrazinecarbo-thioamide following the experimental procedure of Preparation 2.

LRMS: m/z 292 (M+1)⁺

Retention time: 3.33 min (method A)

¹H NMR (200 MHz, DMSO-D6) δ ppm 0.9 (t, J=7.2 Hz, 3H) 1.4 (m, 2H) 1.6 (m, 2H) 3.3 (m, 2H) 3.9 (s, 3H) 7.9 (d, J=8.6 Hz, 2H) 8.0 (m, 2H) 8.1 (t, J=5.3 Hz, 1H)

### Preparation 19

### 4-(5-(Butylamino)-1,3,4-thiadiazol-2-yl)-2,6-dimethylphenol

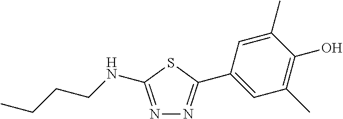

To a stirred suspension of the title product of Preparation 9 (2.0 g, 6.86 mmol) in dichloromethane (20 ml) at −78° C., a 1M BBr₃ solution in dichloromethane (10.3 ml, 10.3 mmol) was added and the mixture was stirred at that temperature for 30 min and then a rt overnight. The reaction mixture was poured onto ice water, diluted with dichloromethane and neutralized with saturated solution of potassium bicarbonate. The organic layer was collected and solvent was removed to yield a solid that was washed with ethyl ether. 1.64 g (87% yield) of the title product were obtained.

LRMS: m/z 278 (M+1)⁺

Retention time: 6.09 min (method B)

¹H NMR (200 MHz, DMSO-D6) δ ppm 0.9 (t, J=7.2 Hz, 3H) 1.4 (m, 2H) 1.6 (m, 2H) 2.2 (s, 6H) 3.3 (m, 2H) 7.3 (s, 2H) 8.0 (s, 1H) 8.8 (s, 1H)

### Preparation 20

### N-Butyl-5-(4-((2,2-dimethyl-1,3-dioxolan-4-yl)methoxy)-3,5-dimethylphenyl)-1,3,4-thiadiazol-2-amine

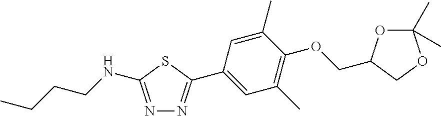

To a stirred solution of the title compound of Preparation 19 (1.0 g, 3.61 mmol) in DMF (15 ml), 60% sodium hydride (0.29 g, 7.25 mmol) was added portionwise and the mixture was stirred at rt for 30 min. Then, 4-(chloromethyl)-2,2-dimethyl-1,3-dioxolane (0.60 ml, 4.22 mmol) was added and the mixture was stirred at 120° C. overnight. Solvent was removed and the residue was partitioned between water and ethyl acetate. The organic layer was washed with water and brine and solvent was finally removed toyield a crude product that was purified according to purification method A. 0.40 g (28% yield) of the title product were obtained.

LRMS: m/z 392 (M+1)⁺

Retention time: 7.12 min (method B)

¹H NMR (200 MHz, DMSO-D6) δ ppm 0.9 (t, J=7.2 Hz, 3H) 1.3 (m, 8H) 1.6 (m, 2H) 2.3 (s, 6H) 3.3 (m, 2H) 3.8 (m, 3H) 4.1 (t, J=7.4 Hz, 1H) 4.4 (m, 1H) 7.4 (s, 2H) 7.9 (t, J=5.7 Hz, 1H)

### Preparation 21

### N-Butyl-2-chloro-N-(5-{4-[(2,2-dimethyl-1,3-dioxolan-4-yl)methoxy]-3,5-dimethyl-phenyl}-1,3,4-thiadiazol-2-yl)benzamide

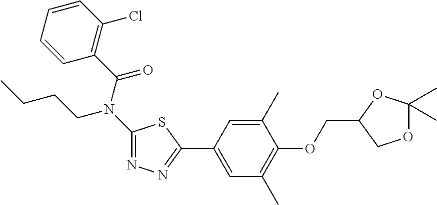

To a stirred suspension of the title compound of Preparation 20 (170 mg, 0.43 mmol) and diisopropylethylamine (0.23 ml, 1.3 mmol) in 1,2-dichloroethane (10 ml), 2-chlorobenzoyl chloride (0.11 ml, 0.87 mmol) and 4-DMAP (27 mg) were added and the mixture was stirred at 60° C. overnight. It was diluted with dichloromethane and washed with potassium bicarbonate, saturated solution of citric acid and brine. Solvent was removed and the crude thus obtained was purified accoriding to purification method A. 111 mg (26% yield) of the title product were obtained.

LRMS: m/z 531 (M+1)⁺

Retention time: 20.80 min (method C)

¹H NMR (200 MHz, DMSO-d₆) δ ppm 0.7 (t, J=7.4 Hz, 3H) 1.2 (m, 2H) 1.3 (d, J=10.7 Hz, 6H) 1.7 (m, 2H) 2.3 (s, 6H) 3.8 (m, 4H) 4.1 (m, 2H) 4.4 (m, 1H) 7.7 (m, 6H)

### Preparation 22

### N-Butyl-5-(2-chloro-6-methoxypyridin-4-yl)-1,3,4-thiadiazol-2-amine

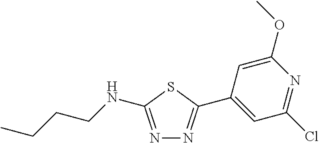

Obtained (70%) from 2-chloro-6-methoxyisonicitinic acid and N-butylhydrazinecarbothioamide following the experimental procedure of Preparation 2.

LRMS: m/z 299 (M+1)⁺

Retention time: 3.61 min (method A)

¹H NMR (200 MHz, DMSO-D6) δ ppm 0.9 (t, J=7.2 Hz, 3H) 1.4 (m, 2H) 1.6 (m, 2H) 3.3 (m, 2H) 3.9 (s, 3H) 7.1 (d, J=1.2 Hz, 1H) 7.4 (d, J=1.2 Hz, 1H) 8.4 (t, J=6.1 Hz, 1H)

### Preparation 23

### N-(2-methoxyethyl)hydrazinecarbothioamide

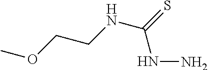

Obtained (89%) from 1-isothiocyanato-2-methoxyethane following the experimental procedure of Preparation 1.

¹H NMR (200 MHz, DMSO-D6) δ ppm 3.2 (s, 3H) 3.4 (m, 2H) 3.6 (m, 2H) 4.5 (s, 2H) 7.8 (s, 1H) 8.7 (s, 1H)

### Preparation 24

### 5-(4-Methoxy-3,5-dimethylphenyl)-N-(2-methoxyethyl)-1,3,4-thiadiazol-2-amine

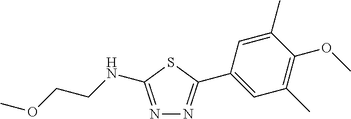

Obtained (23%) from 4-methoxy-3,5-dimethylbenzoic acid and the title compound of Preparation 23 following the experimental procedure of Preparation 2.

LRMS: m/z 294 (M+1)⁺

Retention time: 5.83 min (method B)

¹H NMR (200 MHz, CDCl₃) δ ppm 2.3 (s, 6H) 3.4 (s, 3H) 3.6 (b.s., 4H) 3.8 (s, 3H) 7.5 (s, 2H)

### Preparation 25

N-ethyl-5-(4-methoxy-3,5-dimethylphenyl)-1,3,4-thiadiazol-2-amine

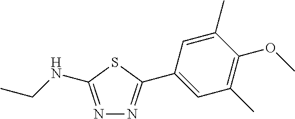

Obtained (83%) from 4-methoxy-3,5-dimethylbenzoic acid and N-ethylhydrazinecarbothioamide following the experimental procedure of Preparation 2.

LRMS: m/z 264 (M+1)⁺

Retention time: 6.13 min (method B)

¹H NMR (200 MHz, CDCl₃) δ ppm 1.3 (t, J=7.1 Hz, 3H) 2.3 (s, 6H) 3.4 (q, J=7.1 Hz, 2H) 3.7 (s, 3H) 5.3 (m, 1H) 7.5 (s, 2H)

### Preparation 26

### N-Butyl-5-(4-(2-methoxyethoxy)-3,5-dimethylphenyl)-1,3,4-thiadiazol-2-amine

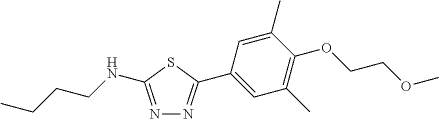

To a stirred solution of the title compound of Preparation 19 (300 mg, 1.08 mmol) in DMF (5 ml), 60% sodium hydride (90 mg, 2.25 mmol) was added portionwise and the mixture was stirred at rt for 30 min. Then, 1-bromo-2-methoxyethane (310 mg, 2.20 mmol) in DMF (2 ml) was added and the mixture was stirred at rt overnight. The reaction mixture was poured onto ice water and is extracted with ethyl acetate twice. The organic layer was washed with water and brine and solvent was finally removed to yield a crude product that was purified according to purification method A. 0.210 g (56% yield) of the title product were obtained.

LRMS: m/z 336 (M+1)⁺

Retention time: 6.75 min (method B)

¹H NMR (200 MHz, DMSO-D6) δ ppm 0.9 (t, J=7.2 Hz, 3H) 1.4 (m, 2H) 1.6 (m, 2H) 2.3 (s, 6H) 3.3 (m, 5H) 3.6 (dd, J=5.6, 3.2 Hz, 2H) 3.9 (dd, J=5.6, 3.2 Hz, 2H) 7.4 (s, 2H) 7.9 (t, J=5.5 Hz, 1H)

### Preparation 27

### N-isopentylhydrazinecarbothioamide

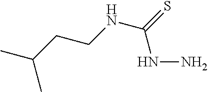

Obtained (86%) from 1-isothiocyanato-3-methylbutane following the experimental procedure of Preparation 1

LRMS: m/z 162 (M+1)⁺

Retention time: 4.95 min (method B)

¹H NMR (200 MHz, DMSO-D6) δ ppm 0.9 (d, J=6.6 Hz, 6H) 1.4 (m, 2H) 1.6 (m, 1H) 3.5 (m, 2H) 4.4 (s, 2H) 7.7 (s, 1H) 8.5 (s, 1H)

### Preparation 28

### N-Isopentyl-5-(4-methoxy-3,5-dimethylphenyl)-1,3,4-thiadiazol-2-amine

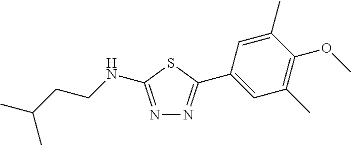

Obtained (72%) from 4-methoxy-3,5-dimethylbenzoic acid and the title compound of Preparation 27 following the experimental procedure of Preparation 2.

LRMS: m/z 306 (M+1)⁺

Retention time: 7.16 min (method B)

### Preparation 29

### N-(3-(Diethylamino)propyl)hydrazinecarbothioamide

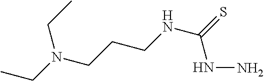

Obtained (96%) from N,N-diethyl-3-isothiocyanatopropan-1-amine following the experimental procedure of Preparation 1.

LRMS: m/z 205 (M+1)⁺

Retention time: 1.07 min (method B)

### Preparation 30

### N1,N1-diethyl-N3-(5-(4-methoxy-3,5-dimethylphenyl)-1,3,4-thiadiazol-2-yl)propane-1,3-diamine

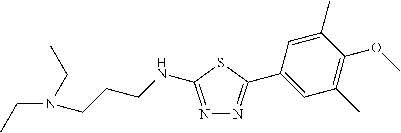

Obtained (20%) from 4-methoxy-3,5-dimethylbenzoic acid and the title compound of Preparation 29 following the experimental procedure of Preparation 2.

LRMS: m/z 349 (M+1)⁺

Retention time: 4.43 min (method B)

### Preparation 31

### N-Butyl-5-(2-chloro-6-methylpyridin-4-yl)-1,3,4-thiadiazol-2-amine

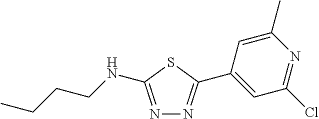

Obtained (30%) from 2-chloro-6-methylisonicitinic acid and N-butylhydrazine-carbothioamide following the experimental procedure of Preparation 2.

LRMS: m/z 283 (M+1)⁺

Retention time: 3.31 min (method A)

### Preparation 32

### N-propylhydrazinecarbothioamide

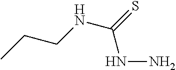

Obtained (88%) from 1-isothiocyanatopropane following the experimental procedure of Preparation 1.

LRMS: m/z 134 (M+1)⁺

Retention time: 3.21 min (method B)

### Preparation 33

### 5-(4-Methoxy-3,5-dimethylphenyl)-N-propyl-1,3,4-thiadiazol-2-amine

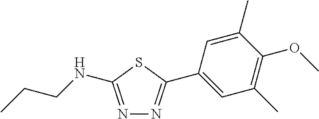

Obtained (76%) from 4-methoxy-3,5-dimethylbenzoic acid and the title compound of Preparation 32 following the experimental procedure of Preparation 2.

LRMS: m/z 278 (M+1)⁺

Retention time: 6.29 min (method B)

### Preparation 34

### tert-Butyl 2-(4-(5-(butylamino)-1,3,4-thiadiazol-2-yl)-2,6-dimethylphenoxy)acetate

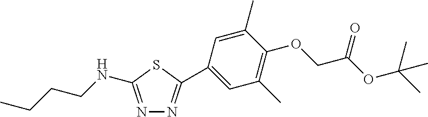

To a 0° C. stirred solution of the title compound of Preparation 19 (300 mg, 1.08 mmol) in DMF (5 ml), 60% sodium hydride (43 mg, 1.79 mmol) was added portionwise and the mixture was stirred at rt for 30 min. Then, tert-butyl 2-bromoacetate (210 mg, 2.20 mmol) in DMF (1.5 ml) was added and the mixture was stirred at that temperature for 30 min. The reaction mixture was poured onto ice water and was extracted with ethyl acetate twice. The organic layer was washed with water and brine and solvent was finally removed to yield a crude product that was purified according to purification method A. 0.160 g (38% yield) of the title product were obtained.

LRMS: m/z 392 (M+1)⁺

Retention time: 7.28 min (method B)

¹H NMR (200 MHz, DMSO-D6) δ ppm 0.9 (m, 3H) 1.4 (m, 2H) 1.5 (s, 9H) 1.6 (m, 2H) 2.3 (s, 6H) 3.3 (m, 2H) 4.4 (s, 2H) 7.4 (s, 2H) 7.9 (t, J=5.1 Hz, 1H)

### Preparation 35

### N-Butyl-5-(2-chloropyridin-4-yl)-1,3,4-thiadiazol-2-amine

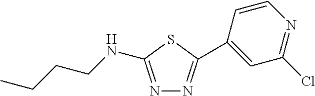

Obtained (18%) from 2-chloroisonicotinic acid and N-butylhydrazinecarbothioamide following the experimental procedure of Preparation 2.

LRMS: m/z 269 (M+1)⁺

Retention time: 3.15 min (method A)

### Preparation 36

### N-Butyl-5-(2-methoxypyridin-4-yl)-1,3,4-thiadiazol-2-amine

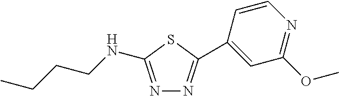

To a stirred solution of the title compound of Preparation 35 in methanol (20 ml), sodium (34 mg, 1.48 mmol) was added portionwise and the mixture was stirred at rt overnight. 10 ml of a solution of 100 mg of sodium in methanol were further added and the mixture was stirred at 60° C. for 4 days. The reaction mixture was poured onto water and extracted with dichloromethane. Solvent was removed to yield 196 mg (73% yield) of the title compound.

LRMS: m/z 265 (M+1)⁺

Retention time: 3.10 min (method A)

### Preparation 37

### N-(Cyclopropylmethyl)hydrazinecarbothioamide

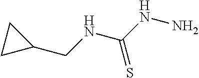

Obtained (88%) from (isothiocyanatomethyl)cyclopropane following the experimental procedure of Preparation 1.

LRMS: m/z 146 (M+1)⁺

Retention time: 3.58 min (method B)

### Preparation 38

### N-(Cyclopropylmethyl)-5-(4-methoxy-3,5-dimethylphenyl)-1,3,4-thiadiazol-2-amine

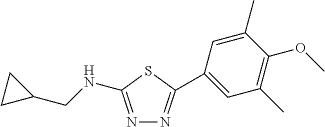

Obtained (100%) from 4-methoxy-3,5-dimethylbenzoic acid and the title compound of Preparation 37 following the experimental procedure of Preparation 2.

LRMS: m/z 290 (M+1)⁺

Retention time: 6.35 min (method B)

### Preparation 39

### 6-methoxynicotinic acid

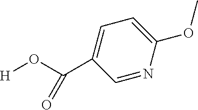

A reaction mixture of 6-metoxynicotinonitrile (4 g, 29.8 mmol) in 60 ml of MeOH and 5M NaOH (60 ml, 300 mmol) was stirred overnight at 100° C. The mixture was concentrated and redissolved in water (50 ml). A 2N solution of HCl was added until pH acid and a solid was formed, filtered and washed with water twice and with hexane twice. 3.04 g of the title compound were obtained as a white solid (yield=66%).

LRMS: m/z 154 (M+1)⁺

Retention time: 2.22 min (method A)

### Preparation 40

### N-butyl-5-(6-methoxypyridin-3-yl)-1,3,4-thiadiazol-2-amine

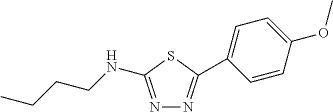

Obtained (31%) from the title compound of Preparation 39 and the title compound of Preparation 1 following the experimental procedure of Preparation 2.

LRMS: m/z 265 (M+1)⁺

Retention time: 3.06 min (method A)

¹H NMR (200 MHz, DMSO-d₆) ppm 0.91 (t, J=8 Hz, 3H), 1.39 (m, 2H), 1.58 (m, 2H), 3.31 (q, J=6 Hz, 2H), 3.90 (s, 3H), 6.93 (d, J=8 Hz, 1H), 7.94 (m, 1H), 8.09 (d, J=8 Hz, 1H), 8.52 (s, 1H)

### Preparation 41

### N-butyl-5-(imidazo[1,2-a]pyridin-6-yl)-1,3,4-thiadiazol-2-amine

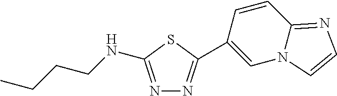

Obtained (47%) from imidazo[1,2-a]pyridine-6-carboxylic acid and the title compound of Preparation 1 following the experimental procedure of Preparation 2.

LRMS: m/z 274 (M+1)⁺

Retention time: 2.08 min (method A)

¹H NMR (200 MHz, DMSO-d₆) ppm 0.98 (t, J=8 Hz, 3H), 1.46 (m, 2H), 1.69 (m, 2H), 3.41 (t, J=8 Hz, 2H), 5.48 (brs, 1H), 7.65 (m, 4H), 8.64 (s, 1H)

### Preparation 42

### Methyl imidazo[1,2-a]pyridine-7-carboxylate

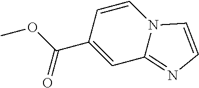

A mixture of 2-bromo-1,1-diethoxyethane (6.20 ml, 41.2 mmol) and HCl 35% (0.82 ml, 26.8 mmol) in water (68 ml) was stirred for 2.5 h, then heated at 80° C. and stirred at this temperature for 1.5 h. The mixture was cooled down to 20° C. and NaHCO₃ (4.49 g, 53.45 mmol) was added in four portions. Finally, methyl 2-aminoisonicotinate (5 g, 32.86 mmol) was added and the reaction mixture stirred at room temperature overnight. The solid formed was filtered, washed with water and dried in the vacuum oven to give 5.15 g of the title compound as a brown solid (yield=89%).

LRMS: m/z 177 (M+1)⁺

Retention time: 1.46 min (method A)

¹H NMR (200 MHz, CHCl₃) ppm 3.97 (s, 3H), 7.27 (s, 1H), 7.42 (d, J=6 Hz, 1H), 7.70 (s, 1H), 7.80 (s, 1H), 8.19 (d, J=6 Hz, 1H), 8.37 (s, 1H)

Yield 89%

### Preparation 43

### Imidazo[1,2-a]pyridine-7-carboxylic acid

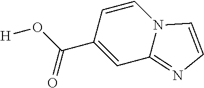

The title compound of Preparation 42 was dissolved in 100 ml of HCl 37%. The reaction mixture was heated at 50° C. for 64 h. After this time the mixture was concentrated to dryness and dried in the vacuum oven to give 1.95 g of the title compound as a solid (yield=100%).

LRMS: m/z 163 (M+1)⁺

Retention time: 0.41 min (method A)

¹H NMR (200 MHz, DMSO-d₆) ppm 7.79 (d, J=8 Hz, 1H), 8.31 (s, 1H), 8.38 (s, 1H), 8.48 (s, 1H), 8.97 (d, J=8 Hz, 1H)

Yield 99.9%

### Preparation 44

### N-butyl-5-(imidazo[1,2-a]pyridin-7-yl)-1,3,4-thiadiazol-2-amine

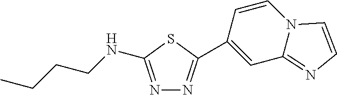

Obtained (76%) from the title compound of Preparation 43 and the title compound of Preparation 1 following the experimental procedure of Preparation 2.

LRMS: m/z 274 (M+1)⁺

Retention time: 1.97 min (method A)

¹H NMR (200 MHz, DMSO-D6) δ ppm 0.9 (t, J=6.2 Hz, 3H) 1.4 (m, 2H) 1.6 (m, 2H) 3.3 (m, 2H) 7.4 (d, J=6.6 Hz, 1H) 7.7 (s, 1H) 7.8 (s, 1H) 8.1 (m, 2H) 8.6 (d, J=5.1 Hz, 1H)

### Preparation 45

### (4-(5-(butylamino)-1,3,4-thiadiazol-2-yl)phenyl)metanol

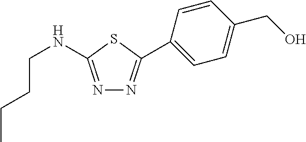

The title compound of preparation 18 (1.5 g, 5.15 mmol) was dissolved in THF (20 ml) and LiAlH₄ (0.2 g, 5.27 mmol) was added portionwise and the mixture stirred overnight at room temperature. Then to the mixture was added water (2 ml), NaOH 32% (4 ml), water (4 ml) and finally ethyl acetate. The organic layer was collected, dried and concentrated to yield the title compound as a yellow solid (75%).

LRMS: m/z 264 (M+1)⁺

Retention time: 2.75 min (method A)

¹H NMR (200 MHz, CDCl₃) δ ppm 0.97 (t, J=7.22 Hz, 3H) 1.45 (td, J=14.74, 7.22 Hz, 2H) 1.60-1.83 (m, 2H) 3.37 (t, J=7.03 Hz, 2H) 4.74 (s, 2H) 5.72 (br. s., 1H) 7.42 (d, J=8.20 Hz, 2H) 7.78 (d, J=8.59 Hz, 2H)

### Preparation 46

### 4-(5-(butylamino)-1,3,4-thiadiazol-2-yl)benzaldehyde

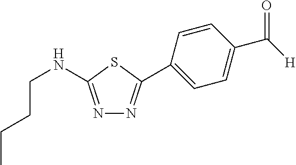

To a solution of oxalyl chloride (0.73 ml, 8.35 mmol) in DCM (20 ml) under argon at −60° C., DMSO (0.89 g, 11.39 mmol) was slowly added keeping the temperature at −60° C. and the mixture stirred at this temperature for 15 min. A suspension of the title compound of preparation 45 (1 g, 3.8 mmol) in 10 ml of DCM was slowly added to this mixture. Finally, diisopropylethylamine was added (4.40 ml, 25.3 mmol) and the mixture stirred at −60° C. for 1 h and at room temperature overnight. Solvent was removed and the residue was solved in ethyl acetate and washed with a 4% solution of NaHCO₃. The organic layer was dried, solvent was removed in vacuo and the crude was purified according to purification method A to yield the title compound as a solid (yield=18%).

LRMS: m/z 262 (M+1)⁺

Retention time: 5.87 min (method B)

¹H NMR (200 MHz, CDCl₃) δ ppm 0.98 (t, J=7.22 Hz, 3H) 1.46 (dd, J=15.03, 7.22 Hz, 2H) 1.72 (t, J=3.51 Hz, 2H) 3.41 (t, J=7.03 Hz, 2H) 7.85-8.05 (m, 4H) 10.04 (s, 1H)

### Preparation 47

### N-butyl-2-fluoro-N-(5-(4-formylphenyl)-1,3,4-thiadiazol-2-yl)benzamide

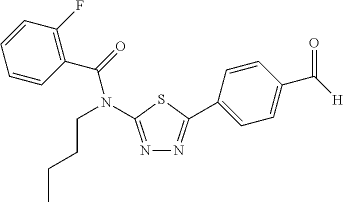

Obtained (18%) from 2-fluorobenzoyl chloride and the title compound of Preparation 46 following the experimental procedure of Preparation 11.

LRMS: m/z 384(M+1)⁺

Retention time: 7.09 min (method B)

### Preparation 48

### (E)-4-(3-methoxy-3-oxoprop-1-enyl)-2-methylbenzoic acid

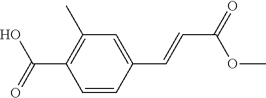

To a mixture of 4-bromo-2-methylbenzoic acid (2 g, 9.3 mmol), methyl acrylate (0.9 g, 10.5 mmol), N,N-dimethylalanine (0.1 g, 0.85 mmol), potassium carbonate (2.6 g, 18.8 mmol) in NMP (50 ml) was added Pd(OAc)₂ (0.1 g, 0.45 mmol) under nitrogen atmosphere. The mixture was stirred overnight at 120° C. The reaction mixture was then concentrated, redissolved in ethyl ether and washed with water. The organic layer was dried and evaporated to give the title compound as an oil (yield=77%).

LRMS: m/z 221(M+1)⁺

Retention time: 5.49 min (method B)

¹H NMR (200 MHz, CDCl₃) δ ppm 2.66 (s, 3H) 3.83 (s, 3H) 6.52 (d, J=16.01 Hz, 1H) 7.36-7.46 (m, 2H) 7.68 (d, J=16.01 Hz, 1H) 8.05 (d, J=7.81 Hz, 1H)

### Preparation 49

### 4-(3-methoxy-3-oxopropyl)-2-methylbenzoic acid

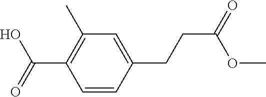

The title compound of preparation 48 (2 g, 9.08 mmol) was dissolved in methanol (50 ml) and after the addition of 10% Pd/C (0.1 g, 0.9 mmol) the mixture was hydrogenated at 20 psi for 1 h. The catalyst was filtered off and the filtrate concentrated to give the title compound as a brown oil (yield=85%) which was used without further purification.

LRMS: m/z 223(M+1)⁺

Retention time: 5.51 min (method B)

### Preparation 50

### Methyl 3-(4-(5-(butylamino)-1,3,4-thiadiazol-2-yl)-3-methylphenyl)propanoate

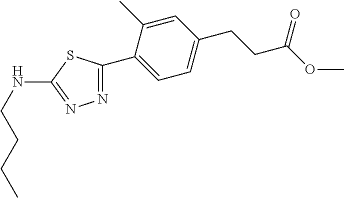

Obtained (15% yield) from the title compound of Preparation 1 and the title compound of Preparation 49 following the experimental procedure of Preparation 2. The crude was purified according to purification method A.

LRMS: m/z 334(M+1)⁺

Retention time: 3.44 min (method A)

### Preparation 51

### Methyl 3-(4-(5-(N-butyl-2-fluorobenzamido)-1,3,4-thiadiazol-2-yl)-3-methylphenyl)-propanoate

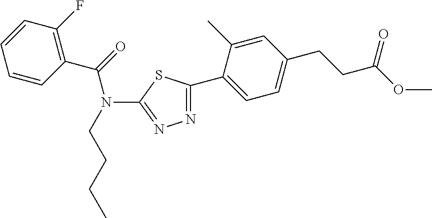

Obtained (60%) from 2-fluorobenzoyl chloride and the title compound of Preparation 50 following the experimental procedure of Preparation 11.

LRMS: m/z 456(M+1)⁺

Retention time: 7.38 min (method B)

### Preparation 52

### 5-(4-methoxy-3,5-dimethylphenyl)-N-phenethyl-1,3,4-thiadiazol-2-amine

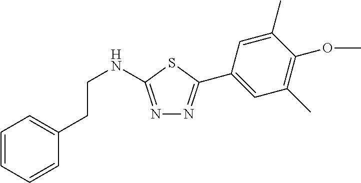

Obtained (97%) from 4-methoxy-3,5-dimethylbenzoic acid and N-phenethylhydrazine-carbothioamide following the experimental procedure of Preparation 2.

LRMS: m/z 340(M+1)⁺

Retention time: 6.94 min (method B)

¹H NMR (200 MHz, DMSO-d₆) δ ppm 2.27 (s, 6H) 2.81-2.99 (m, 2H) 3.56 (d, J=1.56 Hz, 2H) 3.68 (s, 3H) 7.12-7.49 (m, 7H) 7.98 (s, 1H).

### Preparation 53

### 4-cyano-2,6-dimethylphenyl trifluoromethanesulfonate

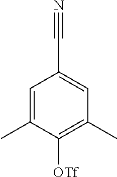

To a mixture of 4-hydroxy-3,5-dimethylbenzonitrile (7 g, 47.5 mmol), pyridine (70 ml) and DCM (12 ml) was added dropwise trifluoromethanesulfonic anhydride (14 g, 49.6 mmol). The reaction mixture was stirred overnight at room temperature and then concentrated to give the title compound as an oil (84% yield).

LRMS: m/z 280(M+1)⁺

Retention time: 7.04 min (method B)

### Preparation 54

### (E)-tert-butyl 3-(4-cyano-2,6-dimethylphenyl)acrylate

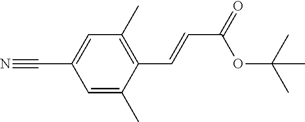

Obtained (38%) from tert-butyl acrylate and the title compound of Preparation 53 following the experimental procedure of Preparation 48.

LRMS: m/z 258(M+1)⁺

Retention time: 7.31 min (method B)

¹H NMR (200 MHz, CDCl₃) δ ppm 1.55 (s, 9H) 2.35 (s, 3H) 6.01 (d, J=16.40 Hz, 1H) 7.34 (s, 2H) 7.52-7.73 (m, 1H)

### Preparation 55

### (E)-4-(2-carboxyvinyl)-3,5-dimethylbenzoic acid

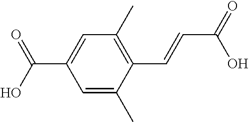

The title compound of Preparation 54 (1 g, 3.89 mmol) was dissolved in ethanol (20 ml) and NaOH 32% (20 ml) was added. The reaction mixture was stirred overnight at 110° C. 5N HCl was added until a solid was formed. This solid was filtered off and dried in the vacuum oven to give a solid as the title compound (yield=89%).

LRMS: m/z 221(M+1)⁺

Retention time: 4.91 min (method B)

### Preparation 56

### 4-(2-carboxyethyl)-3,5-dimethylbenzoic acid

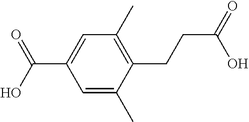

Obtained (84%) from the title compound of Preparation 55 following the experimental procedure of Preparation 49.

LRMS: m/z 223(M+1)⁺

Retention time: 4.95 min (method B)

### Preparation 57

### 3-(4-(5-(butylamino)-1,3,4-thiadiazol-2-yl)-2,6-dimethylphenyl)propanoic acid

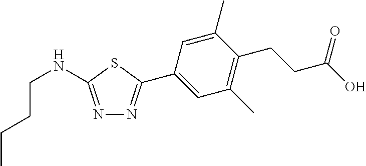

Obtained (40% yield) from the title compound of Preparation 1 and the title compound of Preparation 56 following the experimental procedure of Preparation 2.

LRMS: m/z 334(M+1)⁺

Retention time: 5.86 min (method B)

¹H NMR (200 MHz, DMSO-d₆) δ ppm 0.83-0.98 (m, 3H) 1.22-1.44 (m, 2H) 1.52 (d, J=7.81 Hz, 2H) 2.33 (s, 6H) 2.97 (s, 4H) 3.22 (d, J=5.47 Hz, 2H) 7.60 (s, 2H)

### Preparation 58

### tert-butyl 3-(4-(5-(butylamino)-1,3,4-thiadiazol-2-yl)-2,6-dimethylphenyl)-propanoate

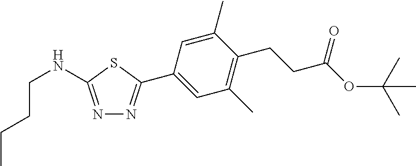

A suspension of the title compound of Preparation 57 (0.2 g, 0.6 mol) in toluene (10 ml) was heated to 100° C. and 1,1-di-tert-butoxy-N,N-dimethylmethanamine was added. The mixture was stirred at this temperature for 2 h and then concentrated to dryness. The crude mixture was dissolved in ethyl acetate and washed with a solution of potassium carbonate. The organic layer was dried and concentrated to give the title compound as a solid (yield=62%).

LRMS: m/z 390(M+1)⁺

Retention time: 7.34 min (method B)

¹H NMR (200 MHz, CDCl₃) δ ppm 0.96 (t, J=7.03 Hz, 3H) 1.45 (d, J=6.25 Hz, 2H) 1.59 (s, 9H) 1.66 (d, J=7.42 Hz, 2H) 2.37 (s, 6H) 2.94-3.14 (m, 4H) 3.31 (br. s., 2H) 7.64 (s, 2H)

### Preparation 59

### tert-butyl 3-(4-(5-(N-butyl-2-fluorobenzamido)-1,3,4-thiadiazol-2-yl)-2,6 dimethyl-phenyl)propanoate

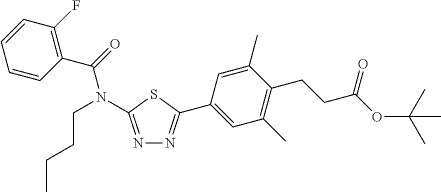

Obtained (20%) from 2-fluorobenzoyl chloride and the title compound of Preparation 58 following the experimental procedure of Preparation 11.

LRMS: m/z 512 (M+1)⁺

Retention time: 7.87 min (method B)

### Preparation 60

### (R)—N-butyl-5-(4-(2,2-dimethyl-1,3-dioxolan-4-yloxy)phenyl)-1,3,4-thiadiazol-2-amine

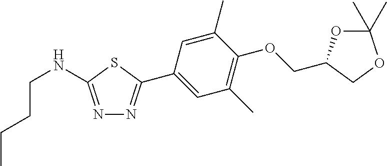

Obtained (12% yield) from the title compound of Preparation 19 (0.35 g, 1.26 mmol) and (R)-4-(chloromethyl)-2,2-dimethyl-1,3-dioxolane following the experimental procedure of Preparation 20. The crude was purified according to purification method A.

LRMS: m/z 392 (M+1)⁺

Retention time: 7.12 min (method B)

### Preparation 61

### (R)—N-butyl-N-(5-(4-(2,2-dimethyl-1,3-dioxolan-4-yloxy)phenyl)-1,3,4-thiadiazol-2-yl)-2-fluorobenzamide

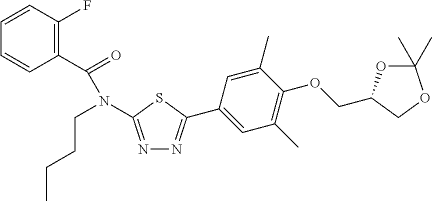

Obtained (20%) from 2-fluorobenzoyl chloride and the title compound of Preparation 60 following the experimental procedure of Preparation 11.

LRMS: m/z 514 (M+1)⁺

Retention time: 7.70 min (method B)

### Preparation 62

### Methyl 4-(5-(N-butyl-2-fluorobenzamido)-1,3,4-thiadiazol-2-yl)benzoate

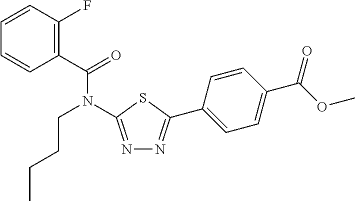

Obtained (41%) from 2-fluorobenzoyl chloride and the title compound of Preparation 18 following the experimental procedure of Preparation 11.

LRMS: m/z 414 (M+1)⁺

Retention time: 7.37 min (method B)

### Preparation 63

### 4-(5-(N-butyl-2-fluorobenzamido)-1-1,3,4-thiadiazol-2-yl)benzoic acid

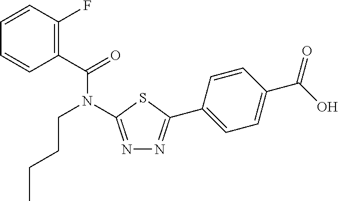

Obtained (20%) from the title compound of Preparation 62 following the experimental procedure of Preparation 43.

LRMS: m/z 400 (M+1)⁺

Retention time: 6.91 min (method B)

¹H NMR (200 MHz, DMSO-D6) δ ppm 0.7 (t, J=7.2 Hz, 3H) 1.2 (m, 2H) 1.7 (m, 2H) 4.1 (m, 2H) 7.5 (m, 2H) 7.7 (m, 2H) 8.1 (m, 4H).

### Preparation 64

### 2-Methoxyisonicotinic Acid

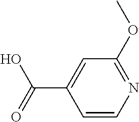

To a solution of 2-chloro-6-methoxyisonicotinic acid (1 g, 5.33 mmol) in methanol (125 mL), Pd/C 10% (0.10 g, 0.09 mmol) and triethylamine (1 mL, 7.21 mmol) were added. The resulting suspension was stirred under hidrogen atmosphere at room temperature for 2 days. Additional catalyst (0.13 g, 0.12 mmol) and triethylamine (1 mL, 7.21 mmol) were needed and mixture stirred under 3 psi of hydrogen atmosphere overnight until completion of reaction. Solid was filtered off, solvent was removed, crude redissolved in dichloromethane, washed with a 4% solution of NaHCO₃, aquous fraction acidified until pH 7-8 and product extracted with diethyl ether to yield 0.82 g (78%) of the title compound.

LRMS: m/z 154(M+1)⁺

Retention time: 3.75 min (method B)

### Preparation 65

### 4-[5-(Butylamino)-1,3,4-thiadiazol-2-yl]-6-chloropyridin-2(1H)-one

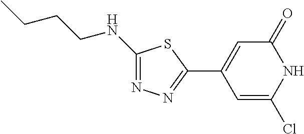

To a suspension of the title compound of Preparation 22 (1.94 g, 6.49 mmol) in acetonitrile (150 mL), sodium iodide (4.90 g, 32.69 mmol) and trimethylsilyl chloride (4.10 mL, 32.42 mmol) were added. The resulting suspension was stirred under nitrogen atmosphere at 40° C. for 3 days. The mixture was cooled to 0° C. and the solid was filtered off, washed with acetonitrile and dried to yield 1.66 g (90%) of the title compound.

LRMS: m/z 285(M+1)⁺

Retention time: 2.86 min (method B)

### Preparation 66

### 4-[5-(Butylamino)-1,3,4-thiadiazol-2-yl]pyridin-2(1H)-one

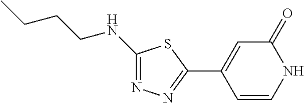

To a mixture of the title compound of Preparation 1 (0.69 g, 4.69 mmol) and the title compound of Preparation 64 (0.72 g, 4.70 mmol) in dioxane (20 mL), phosphorous oxychloride (0.86 mL, 9.42 mmol) was added and the mixture was stirred at 70° C. for 2 hours. The mixture was cooled to room temperature, poured into ice/water and the pH was adjusted to 9 with an aqueous saturated solution of potassium carbonate. It was extracted with ethyl acetate, washed with water and brine, dried, filtered and the solvent was evaporated under vacuum to yield 0.4 g (32%) of the title compound.

LRMS: m/z 251(M+1)⁺

Retention time: 2.40 min (method A)

### Preparation 67

### 4-[5-(Butylamino)-1,3,4-thiadiazol-2-yl]-1-methylpyridin-2(1H)-one

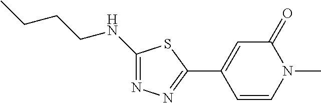

To a solution of the title compound of Preparation 66 (0.40 g, 1.59 mmol) in dimethylformamide (15 mL), cesium carbonate (0.59 g, 1.81 mmol) and methyl iodide (0.12 mL, 1.93 mmol) were added. The resulting suspension was stirred at room temperature for 2 hours, poured into water and extracted with ethyl acetate. The organic layer was washed with water (×2) and brine, dried, filtered and the solvent was evaporated under vacuum. The residue was triturated with a mixture of isopropyl alcohol and diisopropyl ether to yield 110 mg (17%) of the title compound as a solid.

LRMS: m/z 265(M+1)⁺

Retention time: 8.93 min (method C)

¹H NMR (200 MHz, CHLOROFORM-d) δ ppm 0.97 (t, 3H) 1.33-1.58 (m, J=14.88, 7.37, 7.22, 7.22 Hz, 2H) 1.58-1.81 (m, 2H) 3.40 (t, J=7.03 Hz, 2H) 3.57 (s, 3H) 6.01 (s, 1H) 6.72 (d, J=1.95 Hz, 1H) 6.92 (dd, J=7.42, 1.95 Hz, 1H) 7.32 (d, J=7.03 Hz, 1H)

### Preparation 68

### 1-Methyl-6-oxo-1,6-dihydropyridine-3-carboxylic acid

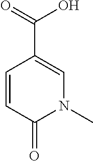

A suspension of sodium hydride (2.70 g, 67.50 mmol) in methanol (60 mL) was stirred under nitrogen atmosphere at room temperature for 5 min. 6-hydroxynicotinic acid (4.70 g, 33.79 mmol) was added, the mixture was stirred at 60° C. for 30 min and then methyl iodide (8.4 mL, 134.93 mmol) was added and the reaction mixture was tirred at 60° C. overnight. 2N sodium hydroxide (20 mL) was added and the mixture was tirred at 60° C. for 30 min. The solvent was evaporated under vacuum, water (200 mL) was added to the residue and it was acidified with 2N hydrochloric acid. The solid was filtered, washed with water and dried and the filtrate was extracted with diethyl ether, dried, filtered and evaporated under vacuum. The resulting crude altogether with the solid was triturated with isopropyl alcohol, filtered, washed with hexanes and dried to give 3.58 g (69%) of the title compound as a solid.

LRMS: m/z 154(M+1)⁺

Retention time: 1.58 min (method A)

¹H NMR (200 MHz, DMSO-d₆) δ ppm 3.49 (s, 3H) 6.40 (d, J=9.76 Hz, 1H) 7.78 (dd, J=9.57, 2.54 Hz, 1H) 8.48 (d, J=2.34 Hz, 1H) 12.78 (br. s., 1H)

### Preparation 69

### 5-[5-(Butylamino)-1,3,4-thiadiazol-2-yl]-1-methylpyridin-2(1H)-one

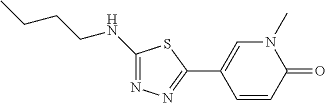

Obtained (36% yield) from the title compound of Preparation 1 and the title compound of Preparation 68 following the procedure described for the synthesis of Preparation 66.

LRMS: m/z 265(M+1)⁺

Retention time: 4.74 min (method B)

### Preparation 70

### tert-Butyl 3-(4-cyano-2,6-dimethylphenyl)propanoate

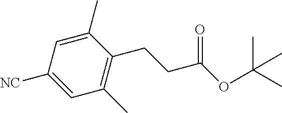

To a solution of the title compound of Preparation 54 (11.5 g, 44.7 mmol) in methanol (350 mL), sodium acetate (5.52 g, 67.3 mmol) and PdCl₂ (2.42 g, 13.65 mmol) were added and the reaction mixture was stirred under hydrogen atmosphere at rt for 1 h. The catalyst was filtered off, the solvent evaporated under vacuum and the crude thus obtained was purified by column chromatography using a 75M Biotage Column and a gradient from 100% hexane to hexane/ethyl acetate 9:1. 3 g (26%) of the title compound were obtained as a white solid.

Retention time: 6.99 min (method B)

### Preparation 71

### 3-(4-(5-Amino-1,3,4-thiadiazol-2-yl)-2,6-dimethylphenyl)propanoic acid

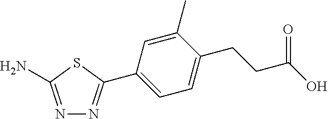

To a mixture of the title compound of Preparation 70 (0.9 g, 3.47 mmol) and hydrazinecarbothioamide (0.35 g, 3.84 mmol), trifluoroacetic acid (1.3 mL, 16.87 mmol) was added and the reaction mixture was stirred at 60° C. for 30 min. Acid was removed and the crude was partitioned between water and ethyl acetate, organic layer was dried, filtered and the solvent evaporated under vacuum. Crude was purified according to purification method A to yield 0.7 g (51%) of the title compound as a white solid.

LRMS: m/z 278(M+1)⁺

Retention time: 2.45 min (method A)

### Preparation 72

### Ethyl 3-[4-(5-amino-1,3,4-thiadiazol-2-yl)-2,6-dimethylphenyl]propanoate

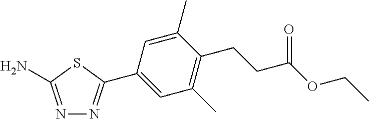

To a solution of the title compound of Preparation 71 (0.7 g, 2.7 mmol) in ethanol (30 mL), concentrated sulphuric acid (1 mL, 18 mmol) was added and the reaction mixture was heated under nitrogen atmosphere at 80° C. for two days. The solvent was evaporated in vacuo, dichloromethane was added, washed with Na₂CO₃ 4% and with water. It was dried, filtered and evaporated in vacuo to yield 0.77 g (99% yield) of the title compound.

LRMS: m/z 306(M+1)⁺

Retention time: 3.32 min (method A)

### Preparation 73

### Ethyl 3-(4-{5-[(2-fluorobenzoyl)amino]-1,3,4-thiadiazol-2-yl}-2,6-dimethylphenyl)propanoate

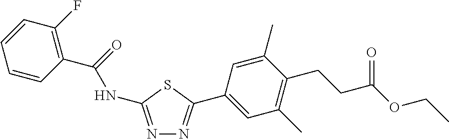

Obtained (73% yield) from the title compound of Preparation 72 and 2-fluorobenzyl chloride following the procedure described for the synthesis of Example 7.

LRMS: m/z 428(M+1)⁺

Retention time: 3.66 min (method A)

### Preparation 74

### Ethyl 3-(4-{5-[ethyl(2-fluorobenzoyl)amino]-1,3,4-thiadiazol-2-yl}-2,6-dimethyl-phenyl)propanoate

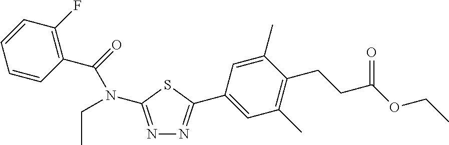

To a solution of the title compound of Preparation 73 (100 mg, 0.23 mmol) in dimethylformamide (3 mL), cesium carbonate (83 mg, 0.25 mmol) was added and the reaction mixture was stirred at room temperature for 10 min. Iodoethane (40 mg, 0.26 mmol) was added and the reaction was stirred at room temperature for 2 hours. The mixture was poured into water and it was extracted with ethyl acetate, dried, filtered and the solvent evaporated under vacuum to yield 42 mg (37%) of the title compound as a brown oil.

LRMS: m/z 456(M+1)⁺

Retention time: 7.80 min (method B)

### Preparation 75

### N-Butyl-N-[5-(4-{[(4R)-2,2-dimethyl-1,3-dioxolan-4-yl]methoxy}-3,5-dimethyl-phenyl)-1,3,4-thiadiazol-2-yl]-2-fluorobenzamide

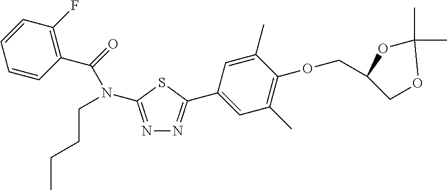

Obtained (40% yield) from the title compound of Preparation 20 and 2-fluorobenzoyl chloride following the procedure described in Preparation 21.

LRMS: m/z 392(M+1)⁺

Retention time: 6.94 min (method B)

### Preparation 76

### N-Butyl-5-(4-vinylphenyl)-1,3,4-thiadiazol-2-amine

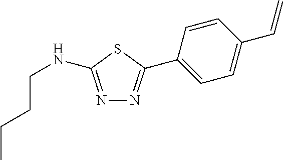

Obtained (34% yield) from the title compound of Preparation 1 and 4-vinylbenzoic acid following the procedure described for the synthesis of Preparation 2.

LRMS: m/z 260(M+1)⁺

Retention time: 6.68 min (method B)

¹H NMR (200 MHz, DMSO-d₆) δ ppm 0.91 (t, J=7.03 Hz, 3H) 1.16-1.48 (m, 2H) 1.46-1.73 (m, 2H) 5.34 (d, J=10.15 Hz, 1H) 5.91 (d, J=17.96 Hz, 1H) 6.78 (dd, J=17.57, 10.93 Hz, 1H) 7.29-7.86 (m, 3H) 7.85-8.12 (m, 1H)

### Preparation 77

### N-Butyl-2-methoxy-N-[5-(4-vinylphenyl)-1,3,4-thiadiazol-2-yl]benzamide

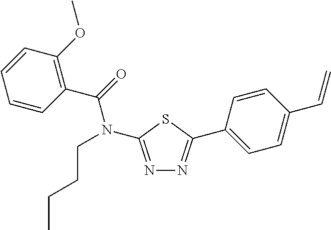

To a solution of the title compound of Preparation 6A2 (1.00 g, 3.86 mmol) in pyridine (10 mL), DMAP (0.24 g, 1.93 mmol) and 2-methoxybenzoyl chloride (0.79 g, 4.63 mmol) were added and the reaction mixture was heated in a microwave system at 110° C. for 35 minutes. The solvent was evaporated in vacuo and the crude was purified following purification method A to give 640 mg (100%) of the title compound.

LRMS: m/z 394(M+1)⁺

Retention time: 7.62 min (method B)

### Preparation 78

### N-Butyl-N-[5-(4-formylphenyl)-1,3,4-thiadiazol-2-yl]-2-methoxybenzamide

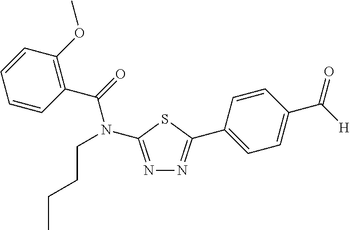

To a suspension of the title compound of Preparation 77 (1.10 g, 2.80 mmol) in acetone (20 mL) and water (2 mL), sodium periodate (1.79 g, 8.37 mmol) and osmium tetroxide (2 mL, 0.31 mmol) were added and the reaction mixture was stirred at room temperature overnight. The reaction mixture was filtered through Celite and the solvent was evaporated under vacuum to give 1.10 g (100%) of the title compound as a yellow gummy solid.

LRMS: m/z 396(M+1)⁺

Retention time: 7.1 min (method B)

### Preparation 79

### 4-{5-[(Cyclopropylmethyl)amino]-1,3,4-thiadiazol-2-yl}-2,6-dimethylphenol

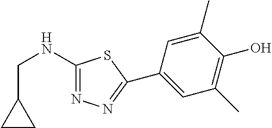

Obtained (87% yield) from the title compound of Preparation 38 following the procedure described in Preparation 19.

LRMS: m/z 276(M+1)⁺

Retention time: 2.9 min (method A)

### Preparation 80

### (S)—N-(cyclopropylmethyl)-5-(4-((2,2-dimethyl-1,3-dioxolan-4-yl)methoxy)-3,5-dimethylphenyl)-1,3,4-thiadiazol-2-amine

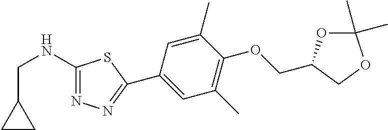

A mixture of the title compound of Preparation 79 (0.50 g, 1.82 mmol), (S)-(+)-(2,2-dimethyl-1,3-dioxolan-4-yl)methanol (0.28 mL, 2.27 mmol), triphenylphosphine (0.60 g, 2.29 mmol) and DIAD (0.45 mL, 2.58 mmol) in THF (2.5 mL) was heated in a microwave system at 80° C. for 40 minutes. The solvent was evaporated under vacuum and the residue was redissolved with dichloromethane, washed with water, brine, dried, filtered and concentrated in vacuo. The crude was purified on a Biotage 40S chromatography column of silica gel using mixtures of hexane/ethyl acetate as eluent to yield 0.61 g (86%) of the title compound.

LRMS: m/z 390(M+1)⁺

Retention time: 6.70 min (method B)

¹H NMR (200 MHz, CHLOROFORM-d) δ ppm 0.18-0.41 (m, 2H) 0.50-0.69 (m, 2H) 1.04-1.32 (m, 1H) 1.44 (d, J=10.93 Hz, 6H) 2.32 (s, 6H) 3.24 (d, J=7.03 Hz, 2H) 3.66-4.04 (m, 3H) 4.06-4.26 (m, 1H) 4.40-4.60 (m, 1H) 5.30 (s, 1H) 7.46 (s, 2H)

### Preparation 81

### N-Butyl-N-[5-(4-vinylphenyl)-1,3,4-thiadiazol-2-yl]nicotinamide

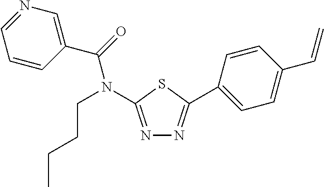

To a solution of nicotinic acid (0.45 g, 1.53 mmol) in DMF (20 mL), EDC.HCl (670 mg, 3.50 mmol) and HOBt (470 mg, 3.48 mmol) were added and the reaction mixture was stirred at room temperature for 15 min. The title compound of Preparation 76 (0.45 g, 1.73 mmol) was added and the reaction mixture was stirred at room temperature overnight. The solvent was removed in vacuo and the residue was dissolved in ethyl acetate, washed with water, brine, dried, filtered and concentrated under vacuum. The crude was purified on a Biotage 40S chromatography column of silica gel using mixtures of hexane/ethyl acetate as eluent to yield 0.46 g (69%) of the title compound as a white solid.

LRMS: m/z 365(M+1)⁺

Retention time: 6.94 min (method B)

### Preparation 82

### N-Butyl-N-[5-(4-formylphenyl)-1,3,4-thiadiazol-2-yl]nicotinamide

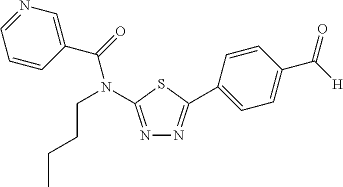

Obtained (100% yield) from the title compound of Preparation 81 following the procedure described in Preparation 78.

LRMS: m/z 367(M+1)⁺

Retention time: 6.25 min (method B)

### Preparation 83

### N-Ethylhydrazinecarbothioamide

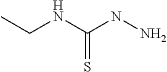

Obtained (95% yield) from 1-isothiocyanatoethane following the experimental procedure of Preparation 1.

LRMS: m/z 120(M+1)⁺

Retention time: 1.27 min (method B)

### Preparation 84

### N-Ethyl-5-(4-vinylphenyl)-1,3,4-thiadiazol-2-amine

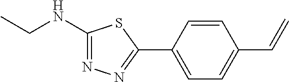

Obtained (20% yield) from the title compound of Preparation 83 and 4-vinylbenzoic acid following the procedure described for the synthesis of Preparation 2.

LRMS: m/z 232(M+1)⁺

Retention time: 5.97 min (method B)

### Preparation 85

### N-Ethyl-2-fluoro-N-[5-(4-vinylphenyl)-1,3,4-thiadiazol-2-yl]benzamide

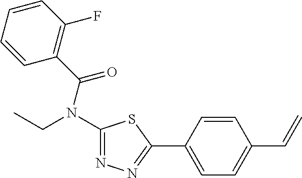

To a 0° C. cooled solution of the title compound of Preparation 84 (0.36 g, 1.56 mmol) in THF (20 mL), sodium hydride (38 mg, 1.58 mmol) was added portionwise and the resulting mixture was stirred for 30 min. 2-fluorobenzoyl chloride (278 mg, 1.75 mmol) was added dropwise and the mixture was stirred at room temperature for 2 hours. 2N hydrochloric acid (5 mL) and ethyl acetate were added, the organic layer was washed with water, brine, dried, filtered and the solvent was removed in vacuo. The crude was purified through purification method A to yield 300 mg (49%) of the title compound as a solid.

LRMS: m/z 354(M+1)⁺

Retention time: 7.12 min (method B)

### Preparation 86

### N-Ethyl-2-fluoro-N-[5-(4-formylphenyl)-1,3,4-thiadiazol-2-yl]benzamide

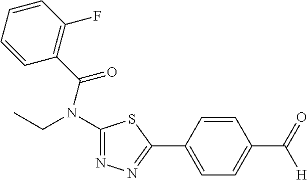

Obtained (100% yield) from the title compound of Preparation 85 following the procedure described in Preparation 78.

LRMS: m/z 356(M+1)⁺

Retention time: 6.48 min (method B)

### Preparation 87

### tert-Butyl N-(4-{5-[butyl(2-fluorobenzoyl)amino]-1,3,4-thiadiazol-2-yl}benzoyl)-b-alaninate

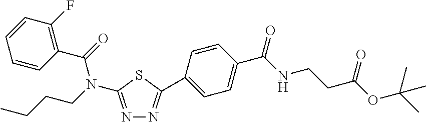

Obtained (85% yield) from the title compound of Preparation 63 and tert-butil-3-aminopropanoate following the experimental procedure of Preparation 81.

LRMS: m/z 527(M+1)⁺

Retention time: 3.71 min (method B)

### Preparation 88

### N-(Cyclopropylmethyl)-5-(4-{[(4R)-2,2-dimethyl-1,3-dioxolan-4-yl]methoxy}-3,5-dimethylphenyl)-1,3,4-thiadiazol-2-amine

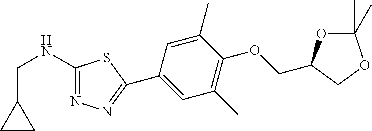

A mixture of the title compound of Preparation 79 (0.50 g, 1.82 mmol), (R)-(+)-(2,2-dimethyl-1,3-dioxolan-4-yl)methanol (482 mg, 3.65 mmol), supported triphenylphosphine (2.3 g, 1.6 mmol/g, 3.67 mmol) and DIAD (1 mL, 3.67 mmol) in THF (15 mL) was heated in a microwave system at 80° C. for 40 minutes. The solvent was evaporated under vacuum and the residue was redissolved with dichloromethane, washed with water, brine, dried, filtered and concentrated in vacuo. The crude was purified on a Biotage 40S chromatography column of silica gel using mixtures of hexane/ethyl acetate as eluent to yield 0.36 g (51%) of the title compound.

LRMS: m/z 390(M+1)⁺

Retention time: 6.6 min (method B)

¹H NMR (200 MHz, CHLOROFORM-d) δ ppm 0.15-0.43 (m, 2H) 0.49-0.73 (m, 2H) 0.97-1.32 (m, 2H) 1.44 (d, J=10.93 Hz, 6H) 2.32 (s, 6H) 3.24 (d, J=7.03 Hz, 2H) 3.69-4.05 (m, 3H) 4.08-4.27 (m, 1H) 4.37-4.60 (m, 1H) 5.34 (br. s., 1H) 7.46 (s, 2H)

### Preparation 89

### N-Propyl-5-(4-vinylphenyl)-1,3,4-thiadiazol-2-amine

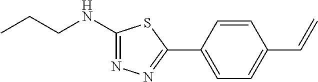

Obtained (17% yield) from the title compound of Preparation 32 and 4-vinylbenzoic acid following the procedure described in Preparation 2.

LRMS: m/z 258(M+1)⁺

Retention time: 6.42 min (method B)

### Preparation 90

### 2-Fluoro-N-propyl-N-[5-(4-vinylphenyl)-1,3,4-thiadiazol-2-yl]benzamide

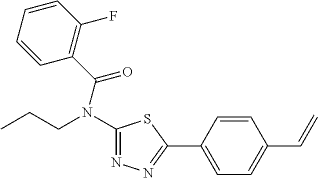

Obtained (37% yield) from the title compound of Preparation 89 and 2-fluorobenzoyl chloride following the procedure described for the synthesis of Preparation 85.

LRMS: m/z 380(M+1)⁺

Retention time: 7.39 min (method B)

### Preparation 91

### 2-Fluoro-N-[5-(4-formylphenyl)-1,3,4-thiadiazol-2-yl]-N-propylbenzamide

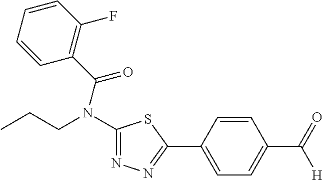

Obtained (38% yield) from the title compound of Preparation 90 following the procedure described in Preparation 78.

LRMS: m/z 370(M+1)⁺

Retention time: 6.78 min (method B)

### Preparation 92

### tert-Butyl (3R)-3-[(4-{5-[butyl(2-fluorobenzoyl)amino]-1,3,4-thiadiazol-2-yl}benzoyl)amino]butanoate

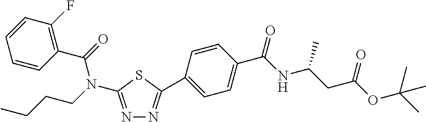

Obtained (42% yield) from the title compound of Preparation 63 and tert-butyl (3R)-3-aminobutanoate following the procedure described in Preparation 81.

LRMS: m/z 541(M+1)⁺

Retention time: 3.75 (method B)

### Preparation 93

### tert-Butyl (3S)-3-[(4-{5-[butyl(2-fluorobenzoyl)amino]-1,3,4-thiadiazol-2-yl}benzoyl)amino]butanoate

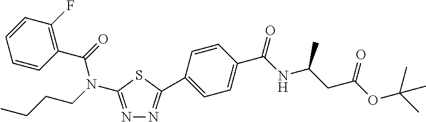

Obtained (100% yield) from the title compound of 63 and tert-butyl (3S)-3-aminobutanoate following the procedure described in Preparation 81.

LRMS: m/z 541(M+1)⁺

Retention time: 17.50 min (method C)

### Preparation 94

### 5-(4-Allylphenyl)-N-butyl-1,3,4-thiadiazol-2-amine

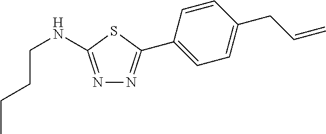

Obtained (70% yield) from the title compound of Preparation 1 and 4-allylbenzoic acid following the procedure described in Preparation 2.

LRMS: m/z 274(M+1)⁺

Retention time: 7.07 min (method B)

¹H NMR (200 MHz, CHLOROFORM-d) δ ppm 0.97 (t, J=7.22 Hz, 3H) 1.30-1.56 (m, J=14.74, 7.27, 7.27, 7.03 Hz, 2H) 1.58-1.79 (m, J=7.22, 7.22, 7.03, 6.64 Hz, 2H) 1.91 (d, J=5.47 Hz, 2H) 3.37 (t, J=6.83 Hz, 2H) 5.47 (br. s., 1H) 6.14-6.61 (m, 2H) 7.12-7.60 (m, 2H) 7.72 (d, J=8.20 Hz, 1H)

### Preparation 95

### N-[5-(4-Allylphenyl)-1,3,4-thiadiazol-2-yl]-N-butyl-2-fluorobenzamide

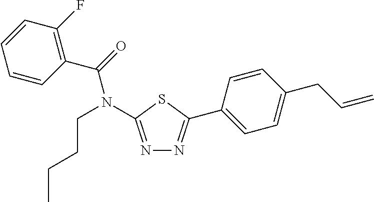

Obtained (9% yield) from the title compound of Preparation 94 and 2-fluorobenzoyl chloride following the procedure described in Preparation 85.

LRMS: m/z 396(M+1)⁺

Retention time: 7.68 min (method B)

### Preparation 96

### N-Butyl-2-fluoro-N-{5-[4-(2-oxoethyl)phenyl]-1,3,4-thiadiazol-2-yl}benzamide

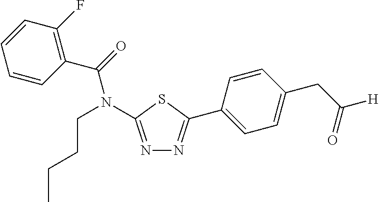

Obtained (60%) from the title compound of Preparation 95 following the procedure described in Preparation 78.

LRMS: m/z 398(M+1)⁺

Retention time: 6.7 min (method B)

### Preparation 97

### N-(Cyclopropylmethyl)-5-(4-vinylphenyl)-1,3,4-thiadiazol-2-amine

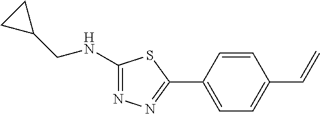

Obtained (78% yield) from the title compound of Preparation 37 and 4-vinylbenzoic acid following the procedure described in Preparation 2.

LRMS: m/z 258(M+1)⁺

Retention time: 6.42 min (method B)

### Preparation 98

### N-(Cyclopropylmethyl)-2-fluoro-N-[5-(4-vinylphenyl)-1,3,4-thiadiazol-2-yl]benzamide

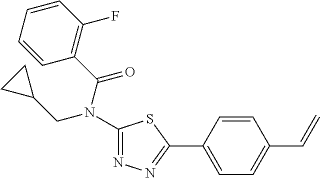

Obtained (37% yield) from the title compound of Preparation 97 and 2-fluorobenzoyl chloride following the procedure described in Preparation 85.

LRMS: m/z 380(M+1)⁺

Retention time: 7.42 min (method B)

### Preparation 99

### N-(Cyclopropylmethyl)-2-fluoro-N-[5-(4-formylphenyl)-1,3,4-thiadiazol-2-yl]benzamide

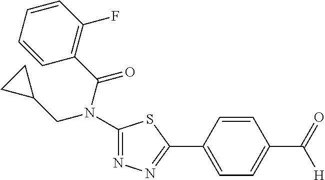

Obtained (83% yield) from the title compound of Preparation 98 following the procedure described in Preparation 78.

LRMS: m/z 382(M+1)⁺

Retention time: 6.80 min (method B)

### Preparation 100

### N-(3-Methylbutyl)-5-(4-vinylphenyl)-1,3,4-thiadiazol-2-amine

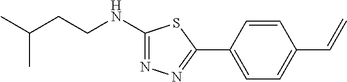

Obtained (45% yield) from the title compound of Preparation 27 and 4-vinylbenzoic acid following the experimental procedure described in Preparation 2.

LRMS: m/z 274(M+1)⁺

Retention time: 7.29 min (method B)

### Preparation 101

### 2-Fluoro-N-(3-methylbutyl)-N-[5-(4-vinylphenyl)-1,3,4-thiadiazol-2-yl]benzamide

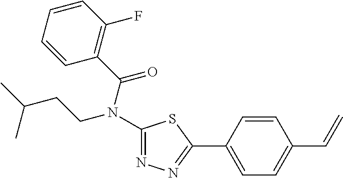

Obtained (48% yield) from the title compound of Preparation 100 and 2-fluorobenzoyl chloride following the procedure described in Preparation 85.

LRMS: m/z 396(M+1)⁺

Retention time: 7.64 min (method B)

### Preparation 102

### 2-Fluoro-N-[5-(4-formylphenyl)-1,3,4-thiadiazol-2-yl]-N-(3-methylbutyl)benzamide

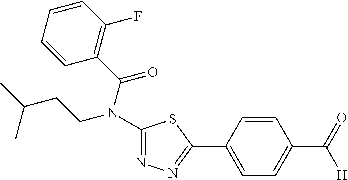

Obtained (46% yield) from the title compound of Preparation 101 following the procedure described in Preparation 78.

LRMS: m/z 398(M4-1)⁺

Retention time: 7.26 min (method B)

### Preparation 103

### N-Methyl-5-(4-vinylphenyl)-1,3,4-thiadiazol-2-amine

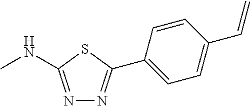

Obtained (66% yield) from N-methylhydrazinecarbothioamide and 4-vinylbenzoic acid following the experimental procedure described in Preparation 2.

Retention time: 7.01 min (method B)

### Preparation 104

### 2-Fluoro-N-methyl-N-[5-(4-vinylphenyl)-1,3,4-thiadiazol-2-yl]benzamide

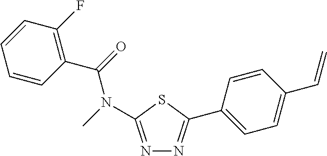

Obtained (37% yield) from the title compound of Preparation 103 and 2-fluorobenzoyl chloride following the procedure described in Preparation 85.

LRMS: m/z 340(M+1)⁺

Retention time: 7.03 min (method B)

### Preparation 105

### 2-Fluoro-N-[5-(4-formylphenyl)-1,3,4-thiadiazol-2-yl]-N-methylbenzamide

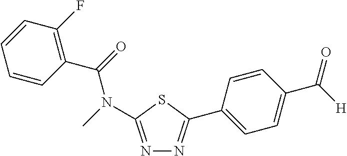

Obtained (29% yield) from the title compound of Preparation 18A1 following the procedure described in Preparation 78.

LRMS: m/z 342(M+1)⁺

Retention time: 6.26 min (method B)

### Preparation 106

### Ethyl 4-bromo-2-methylbenzoate

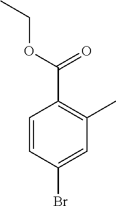

To a solution of 4-bromo-2-methylbenzoic acid (35.4 g, 0.16 mol) in ethanol (250 mL), concentrated sulphuric acid (8.77 mL, 0.16 mol) was added and the reaction mixture was heated under nitrogen atmosphere at 100° C. overnight. The solvent was evaporated in vacuo; ethyl acetate was added, washed with water, 2N sodium hydroxide (×2) and again with water. It was dried, filtered and evaporated in vacuo to yield 38.30 g (92% yields) of the title compound as colourless oil.

LRMS: m/z 243(M+1)⁺

Retention time: 7.08 min (method B)

### Preparation 107

### Ethyl 2-methyl-4-vinylbenzoate

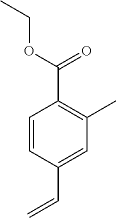

To a degassed solution of the title compound of Preparation 106 (38.3 g, 0.16 mol) in DMF (320 mL), tributyl(vinyl)stannane (58.70 g, 0.19 mol) was added. Tetrakis(triphenylphosphine)palladium (18.21 g, 0.02 mol) was added and the mixture was stirred under nitrogen atmosphere at 90° C. overnight. The solvent was partially evaporated in vacuo, ethyl acetate and water were added, the organic phase was separated and the organic layer was extracted with ethyl acetate (×2). The organic layers were washed with brine, dried, filtered and the solvent was evaporated under vacuum to yield 8.69 g (29% yield) of the title compound.

LRMS: m/z 191(M+1)⁺

Retention time: 6.90 min (method B)

### Preparation 108

### 2-Methyl-4-vinylbenzoic acid

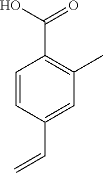

Obtained (83% yield) from the title compound of Preparation 107 following the procedure described in Preparation 55.

LRMS: m/z 161(M−1)⁻

Retention time: 5.92 min (method B)

¹H NMR (200 MHz, DMSO-d₆) δ ppm 2.56 (s, 3H) 5.29 (d, 1H) 5.86 (d, 1H) 2.25 (s, 6H) 6.77 (dd, 2H) 7.4-7.95 (m, 3H) 12.8 (s, 1H)

### Preparation 109

### N-Butyl-5-(2-methyl-4-vinylphenyl)-1,3,4-thiadiazol-2-amine

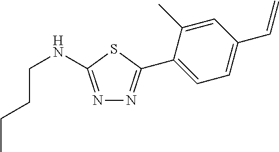

Obtained (66% yield) from the title compound of Preparation 108 and Preparation 1 following the experimental procedure described in Preparation 2.

LRMS: m/z 274(M+1)⁺

Retention time: 6.97 min (method B)

### Preparation 110

### N-Butyl-2-fluoro-N-[5-(2-methyl-4-vinylphenyl)-1,3,4-thiadiazol-2-yl]benzamide

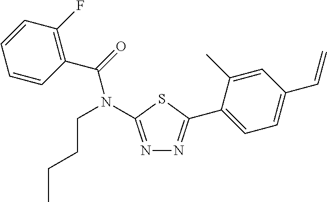

Obtained (37% yield) from the title compound of Preparation 109 and 2-fluorobenzoyl chloride following the procedure described in Preparation 85.

LRMS: m/z 396(M+1)⁺

Retention time: 7.67 min (method B)

### Preparation 111

### N-Butyl-2-fluoro-N-[5-(4-formyl-2-methylphenyl)-1,3,4-thiadiazol-2-yl]benzamide

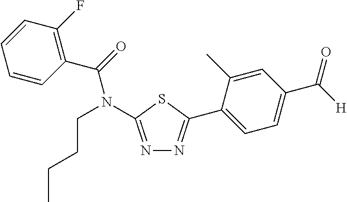

Obtained (84% yield) from the title compound of Preparation 110 following the procedure described in Preparation 78.

LRMS: m/z 398(M+1)⁺

Retention time: 7.15 min (method B)

### Preparation 112

### tert-Butyl 4-[(4-{5-[butyl(2-fluorobenzoyl)amino]-1,3,4-thiadiazol-2-yl}benzoyl)-amino]butanoate

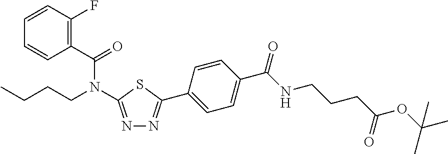

Obtained (77% yield) from the title compound of Preparation 63 and tert-buty-4-aminobutanoate following the procedure described in Preparation 81.

LRMS: m/z 541(M+1)⁺

Retention time: 7.31 min (method B)

### Preparation 113

### N-(2-Methoxyethyl)-5-(4-vinylphenyl)-1,3,4-thiadiazol-2-amine

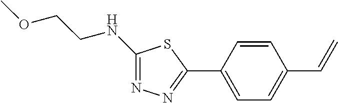

Obtained (66% yield) from the title compound of Preparation 23 and 4-vinylbenzoic acid following the experimental procedure described in Preparation 2.

LRMS: m/z 262(M+1)⁺

Retention time: 5.78 min (method B)

### Preparation 114

### 2-Fluoro-N-(2-methoxyethyl)-N-[5-(4-vinylphenyl)-1,3,4-thiadiazol-2-yl]benzamide

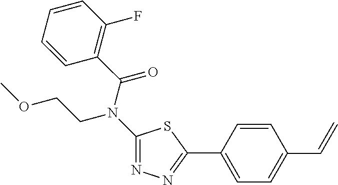

Obtained (13% yield) from the title compound of Preparation 113 and 2-fluorobenzoyl chloride following the procedure described in Preparation 85.

LRMS: m/z 384(M+1)⁺

Retention time: 7.13 (method B)

### Preparation 115

### 2-Fluoro-N-[5-(4-formylphenyl)-1,3,4-thiadiazol-2-yl]-N-(2-methoxyethyl)benzamide

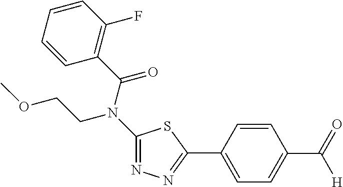

Obtained (62% yield) from the title compound of Preparation 114 following the procedure described in Preparation 78.

LRMS: m/z 3861)⁺

Retention time: 6.47 (method B)

### Preparation 116

### N-Butyl-N-[5-(4-formylphenyl)-1,3,4-thiadiazol-2-yl]-2-phenylacetamide

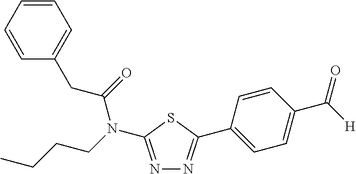

Obtained (67% yield) from the title compound of Preparation 46 and 2-phenylacetyl chloride following the procedure described in Example 7.

LRMS: m/z 380(M+1)⁺

Retention time: 7.17 min (method B)

### Preparation 117

### N-Butyl-2,6-difluoro-N-[5-(4-formylphenyl)-1,3,4-thiadiazol-2-yl]benzamide

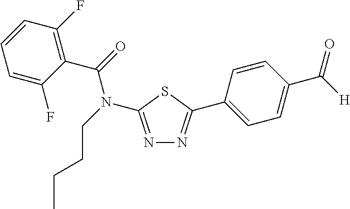

Obtained (68% yield) from the title compound of Preparation 46 and 2,6-difluorophenyl chloride following the procedure described in Example 7.

### Preparation 118

### N-Butyl-2-(2-fluorophenyl)-N-[5-(4-formylphenyl)-1,3,4-thiadiazol-2-yl]acetamide

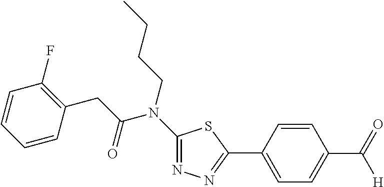

Obtained (100% yield) from the title compound of Preparation 46 and 2-fluorophenylacetyl chloride following the procedure described in Preparation 85.

LRMS: m/z 398(M+1)⁺

Retention time: 7.15 min (method B)

### Preparation 119

### N-(cyclopropylmethyl)-2-methyl-N-[5-(4-vinylphenyl)-1,3,4-thiadiazol-2-yl]benzamide

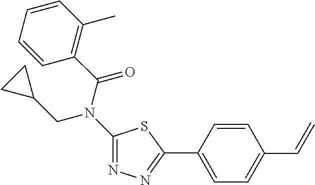

Obtained (46% yield) from the title compound of Preparation 97 and 2-methylbenzoyl chloride following the procedure described in Preparation 85.

LRMS: m/z 376(M+1)⁺

Retention time: 7.45 min (method B)

### Preparation 120

### N-(Cyclopropylmethyl)-N-[5-(4-formylphenyl)-1,3,4-thiadiazol-2-yl]-2-methylbenzamide

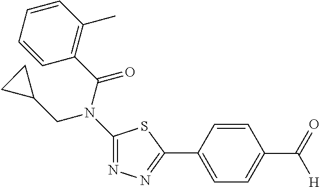

Obtained (48% yield) from the title compound of Preparation 119 following the procedure described in Preparation 78.

LRMS: m/z 378(M+1)⁺

Retention time: 7.05 min (method B)

### Preparation 121

### N-(Cyclopropylmethyl)-2-phenyl-N-[5-(4-vinylphenyl)-1,3,4-thiadiazol-2-yl]acetamide

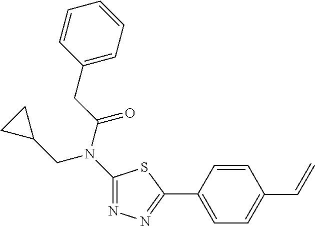

Obtained (90% yield) from the title compound of Preparation 97 and 2-phenylacetyl chloride following the procedure described in Preparation 85.

LRMS: m/z 376(M+1)⁺

Retention time: 7.55 min (method B)

### Preparation 122

### N-(Cyclopropylmethyl)-N-[5-(4-formylphenyl)-1,3,4-thiadiazol-2-yl]-2-phenylacetamide

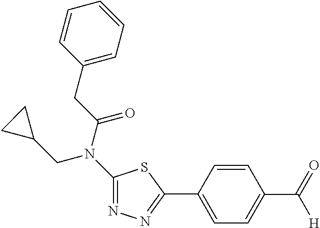

Obtained (29% yield) from the title compound of Preparation 121 following the procedure described in Preparation 78.

LRMS: m/z 378(M+1)⁺

Retention time: 6.90 min (method B)

### Preparation 123

### N-(Cyclopropylmethyl)-4-methoxy-N-[5-(4-vinylphenyl)-1,3,4-thiadiazol-2-yl]benzamide

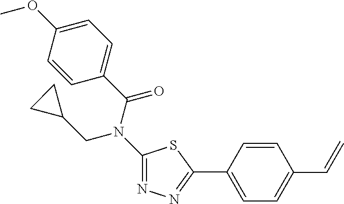

Obtained (74% yield) from the title compound of Preparation 97 and 2-methoxybenzoyl chloride following the procedure described in Preparation 85.

LRMS: m/z 392(M+1)⁺

Retention time: 7.42 min (method B)

### Preparation 124

### N-(Cyclopropylmethyl)-N-[5-(4-formylphenyl)-1,3,4-thiadiazol-2-yl]-4-methoxybenzamide

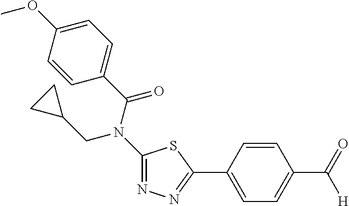

Obtained (29% yield) from the title compound of Preparation 123 following the procedure described in Preparation 78.

LRMS: m/z 394(M+1)⁺

Retention time: 6.88 min (method B)

### Preparation 125

### N-Butyl-N-[5-(4-formylphenyl)-1,3,4-thiadiazol-2-yl]benzamide

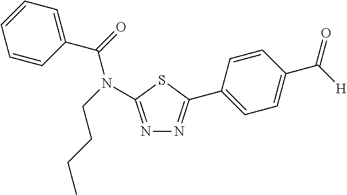

Obtained (9% yield) from the title compound of Preparation 46 and benzoyl chloride following the procedure described in Example 7.

LRMS: m/z 366(M+1)⁺

Retention time: 7.07 min (method B)

### Preparation 126

### 4-Cyano-3-methylbenzoic acid

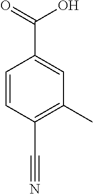

To a −78° C. cooled solution of 4-bromo-2-methylbenzonitrile (4.00 g, 20.40 mmol) in THF (200 mL) under nitrogen atmosphere, 2.5M solution of n-BuLi in hexanes (10 mL, 25 mmol) was slowly added and the resulting solution left to stir at −78° C. for 1 hour. This solution was slowly poured into a mixture of solid CO2 (10 g) in THF (100 mL) and it was stirred at room temperature overnight. The solvent was concentrated in vacuo, water was added, it was extracted with diethyl ether (×3) and the aqueous phase was acidified with 5N hydrochloric acid. The solid was filtered and dried to yield 1.11 g (34% yield) of the title compound.

LRMS: m/z 160(M−1)⁻

Retention time: 4.82 min (method B)

### Preparation 127

### 4-[5-(Butylamino)-1,3,4-thiadiazol-2-yl]-2-methylbenzonitrile

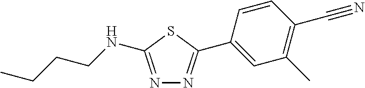

Obtained (96% yield) from the title compound of Preparation 1 and the title compound of Preparation 126 following the procedure described in Preparation 2.

LRMS: m/z 273(M+1)⁺

Retention time: 3.27 min (method A)

### Preparation 128

### 4-[5-(Butylamino)-1,3,4-thiadiazol-2-yl]-2-methylbenzaldehyde

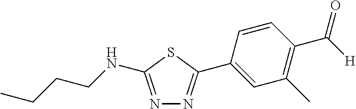

To a solution of the title compound of Preparation 127 (2.40 g, 8.81 mmol) in formic acid (24 mL) and water (7 mL) under nitrogen atmosphere, a nickel alluminium alloy (4.80 g) was added and the reaction mixture was stirred at 100° C. overweekend. More nickel aluminium alloy (4.80 g) was added and the reaction mixture left under stirring at 100° C. for 24 h more. The reaction mixture was filtered through Celite, washed with methanol and concentrated under vacuum. The crude producte was purified on a Merck chromatography column of 70 g silica gel using mixtures of hexane/ethyl acetate as eluent. 0.72 g (25% yield).

LRMS: m/z 276(M+1)⁺

Retention time: 6.15 min (method B)

### Preparation 129

### N-Butyl-2-fluoro-N-[5-(4-formyl-3-methylphenyl)-1,3,4-thiadiazol-2-yl]benzamide

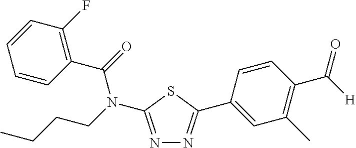

Obtained (28% yield) from the title compound of Preparation 128 and 2-fluorobenzoyl chloride following the procedure described in Example 7.

LRMS: m/z 398(M+1)⁺

Retention time: 7.30 min (method B)

### Preparation 130

### N-Butyl-2-chloro-N-[5-(4-formylphenyl)-1,3,4-thiadiazol-2-yl]benzamide

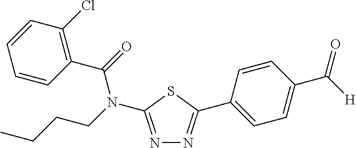

Obtained (26% yield) from the title compound of Preparation 46 and 2-chlorobenzoyl chloride following the procedure described in Preparation 85.

LRMS: m/z 400 (M+1)⁺

Retention time: 7.28 min (method B)

### Preparation 131

### N-Ethyl-2-phenyl-N-[5-(4-vinylphenyl)-1,3,4-thiadiazol-2-yl]acetamide

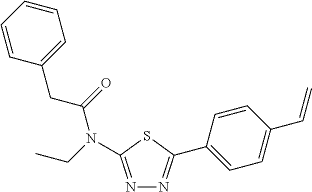

Obtained (15% yield) from the title compound of Preparation 84 and 2-phenylacetyl chloride following the experimental procedure described in Preparation 85.

LRMS: m/z 350(M+1)⁺

Retention time: 7.18 min (method B)

### Preparation 132

### N-Ethyl-N-[5-(4-formylphenyl)-1,3,4-thiadiazol-2-yl]-2-phenylacetamide

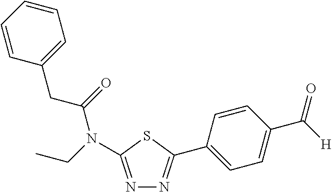

Obtained (39% yield) from the title compound of Preparation 131 following the experimental procedure described in Preparation 78.

LRMS: m/z 352(M+1)⁺

Retention time: 6.57 min (method B)

### Preparation 133

### 4-[5-(Ethylamino)-1,3,4-thiadiazol-2-yl]benzonitrile

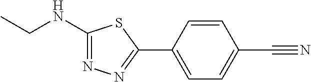

Obtained (19% yield) from the title compound of Preparation 83 and 4-cyanobenzoic acid following the experimental procedure described in Preparation 2.

LRMS: m/z 231(M+1)⁺

Retention time: 5.18 min (method B)

### Preparation 134

### 4-[5-(Ethylamino)-1,3,4-thiadiazol-2-yl]benzaldehyde

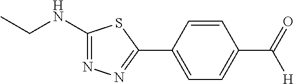

Obtained (61% yield) from the title compound of Preparation 133 following the experimental procedure described in Preparation 128.

LRMS: m/z 234(M+1)⁺

Retention time: 5.00 min (method B)

### Preparation 135

### N-Ethyl-N-[5-(4-formylphenyl)-1,3,4-thiadiazol-2-yl]-2-methoxybenzamide

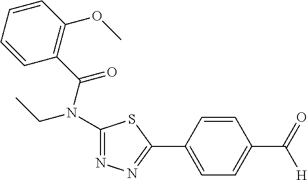

Obtained (82% yield) from the title compound of Preparation 134 and 2-methoxybenzoyl chloride following the procedure described in Example 7.

LRMS: m/z 368(M+1)⁺

Retention time: 6.48 min (method B)

### Preparation 136

### 2-Chloro-N-ethyl-N-[5-(4-vinylphenyl)-1,3,4-thiadiazol-2-yl]benzamide

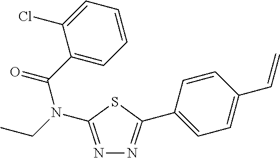

Obtained (8% yield) from the title compound of Preparation 84 and 2-chlorobenzoyl chloride following the experimental procedure described in Preparation 85.

LRMS: m/z 370(M+1)⁺

Retention time: 7.35 min (method B)

### Preparation 137

### 2-Chloro-N-ethyl-N-[5-(4-formylphenyl)-1,3,4-thiadiazol-2-yl]benzamide

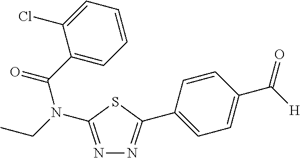

Obtained (100% yield) from the title compound of Preparation 136 following the experimental procedure described in Preparation 78.

LRMS: m/z 372(M+1)⁺

Retention time: 6.80 min (method B)

### Preparation 138

### N-Ethyl-N-[5-(4-formylphenyl)-1,3,4-thiadiazol-2-yl]benzamide

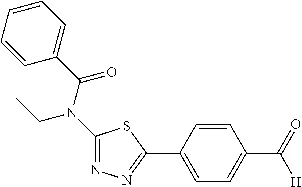

Obtained (65% yield) from the title compound of Preparation 134 and benzoyl chloride following the experimental procedure described in Example 7.

LRMS: m/z 338(M+1)⁺

Retention time: 6.52 min (method B)

### Preparation 139

### N-Butyl-N-[5-(2-methyl-4-vinylphenyl)-1,3,4-thiadiazol-2-yl]-2-phenylacetamide

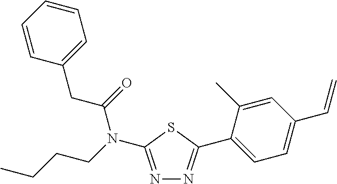

Obtained (18% yield) from the title compound of Preparation 109 and 2-phenylacetyl chloride following the experimental procedure described in Preparation 85.

LRMS: m/z 398(M+1)⁺

Retention time: 18.90 min (method C)

¹H NMR (200 MHz, CHLOROFORM-d) δ ppm 0.99 (t, 3H) 1.27-1.64 (m, J=14.88, 7.37, 7.22, 7.22 Hz, 2H) 1.65-2.03 (m, 2H) 2.62 (s, 3H) 4.05 (s, 2H) 4.16-4.49 (m, 2H) 5.32 (d, J=10.93 Hz, 1H) 5.82 (d, J=17.96 Hz, 1H) 6.71 (dd, J=17.57, 10.93 Hz, 1H) 7.06-7.55 (m, 7H) 7.68 (d, J=8.59 Hz, 1H)

### Preparation 140

### N-Butyl-N-[5-(4-formyl-2-methylphenyl)-1,3,4-thiadiazol-2-yl]-2-phenylacetamide

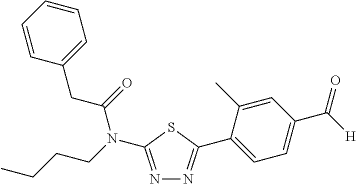

Obtained (90% yield) from the title compound of Preparation 139 following the experimental procedure described in Preparation 78.

LRMS: m/z 394 (M+1)⁺

Retention time: 7.27 min (method B)

### Preparation 141

### 4-Cyano-2-methylbenzoic acid

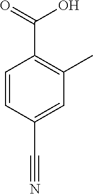

Obtained (55%) yield from 4-bromo-3-methylbenzonitrile following the procedure described in Preparation 126.

LRMS: m/z 162(M+1)⁺

Retention time: 4.70 min (method B)

¹H NMR (300 MHz, DMSO-d₆) δ ppm 2.57 (s, 3H) 7.48-8.13 (m, 3H)

### Preparation 142

### 4-[5-(Butylamino)-1,3,4-thiadiazol-2-yl]-3-methylbenzonitrile

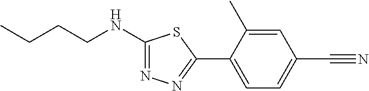

Obtained (80% yield) from the title compound of Preparation 1 and the title compound of Preparation 141 following the experimental procedure described in Preparation 2.

LRMS: m/z 273(M+1)⁺

Retention time: 6.23 min (method B)

¹H NMR (300 MHz, CHLOROFORM-d) δ ppm 0.98 (t, J=7.28 Hz, 3H) 1.35-1.59 (m, J=7.35, 7.35, 7.35, 7.35, 7.14 Hz, 2H) 1.62-1.85 (m, 2H) 2.64 (s, 3H) 3.40 (t, J=7.00 Hz, 2H) 6.24 (br. s., 1H) 7.42-7.86 (m, 3H)

### Preparation 143

### 4-[5-(Butylamino)-1,3,4-thiadiazol-2-yl]-3-methylbenzaldehyde

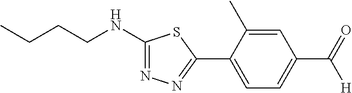

Obtained (57% yield) from the title compound of Preparation 142 following the experimental procedure described in Preparation 128.

LRMS: m/z 276(M+1)⁺

Retention time: 6.12 min (method B)

¹H NMR (300 MHz, CHLOROFORM-d) δ ppm 0.98 (t, J=7.28 Hz, 3H) 1.33-1.57 (m, J=15.00, 7.45, 7.31, 7.31 Hz, 2H) 1.61-1.82 (m, 2H) 2.69 (s, 3H) 3.40 (t, J=7.14 Hz, 2H) 6.34 (br. s., 1H) 7.66-7.85 (m, 3H)

### Preparation 144

### N-Butyl-2-(2-chlorophenyl)-N-[5-(4-formyl-2-methylphenyl)-1,3,4-thiadiazol-2-yl]acetamide

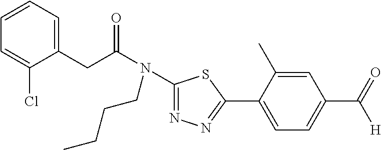

Obtained (34% yield) from the title compound of Preparation 143 and 2-(2-chlorophenyl)acetyl chloride following the experimental procedure described in Example 7.

LRMS: m/z 428(M+1)⁺

Retention time: 7.40 min (method B)

### Preparation 145

### N-Ethyl-3-fluoro-N-[5-(4-formylphenyl)-1,3,4-thiadiazol-2-yl]benzamide

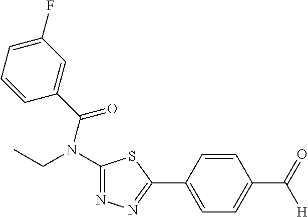

Obtained (49% yield) from the title compound of Preparation 134 and 2-fluorobenzoyl chloride following the experimental procedure described in Example 7.

### Preparation 146

### N-butyl-2-chloro-N-[5-(2-methyl-4-vinylphenyl)-1,3,4-thiadiazol-2-yl]benzamide

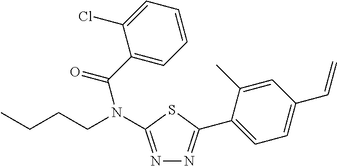

Obtained (52% yield) from the title compound of Preparation 109 and 2-chlorobenzoyl chloride following the procedure described in Preparation 85.

LRMS: m/z 412(M+1)⁺

Retention time: 7.75 min (method B)

¹H NMR (200 MHz, CHLOROFORM-d) δ ppm 0.69-0.90 (m, 3H) 1.04-1.38 (m, 2H) 1.50-1.76 (m, 2H) 2.67 (s, 3H) 4.05-4.59 (m, 2H) 5.34 (dd, J=10.93, 0.78 Hz, 1H) 5.85 (dd, J=17.57, 0.78 Hz, 1H) 6.74 (dd, J=17.57, 10.93 Hz, 1H) 7.30-7.60 (m, 6H) 7.73 (d, J=8.59 Hz, 1H)

### Preparation 147

### N-Butyl-2-chloro-N-[5-(4-formyl-2-methylphenyl)-1,3,4-thiadiazol-2-yl]benzamide

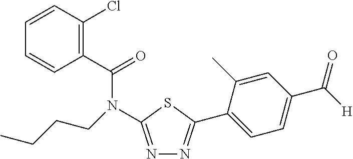

Obtained (85% yield) from the title compound of Preparation 146 following the procedure described in Preparation 78.

LRMS: m/z 414(M+1)⁺

Retention time: 7.33 min (method B)

### Preparation 148

### tert-Butyl[2-(4-{5-[butyl(2-fluorobenzoyl)amino]-1,3,4-thiadiazol-2-yl}-2,6-dimethylphenoxy)ethyl]carbamate

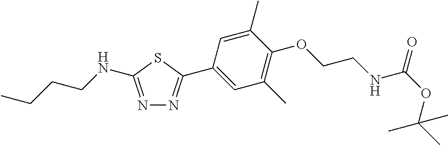

Obtained (36% yield) from the title compound of Preparation 19 and tert-butyl 2-hydroxyethylcarbamate following the procedure described in Preparation 80.

LRMS: m/z 421(M+1)⁺

Retention time: 6.97 min (method B)

¹H NMR (200 MHz, DMSO-d₆) δ ppm 0.91 (t, J=7.22 Hz, 3H) 1.39 (s, 11H) 1.45-1.70 (m, 2H) 2.25 (s, 6H) 3.55-3.88 (m, 2H) 7.06 (t, 1H) 7.85 (t, J=5.47 Hz, 1H)

### Preparation 149

### 4-[5-(Butylamino)-1,3,4-thiadiazol-2-yl]-2,6-dimethylphenyl trifluoromethanesulfonate

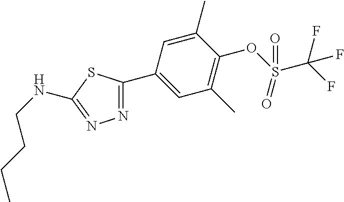

To a solution of the title compound of Preparation 19 (8.59 g, 30.97 mmol) in THF (100 mL), DIEA (8.1 mL, 58.84 mmol) and a solution of N-phenil-bis(trifluorometanosulfonamida) (16.59 g, 58.84 mmol) in THF (125 mL) were added and it was stirred at 75° C. overnight and at 105° C. for 5 days. The reaction mixture was cooled to room temperature, the solvent was evaporated under vacuum and diethyl ether (300 mL) was added to the residue. This solution was washed with 1N sodium hydroxide (5×200 mL), brine, dried, filtered and the solvent concentrated in vacuo to give 11.68 g (92% yield) of the title compound as a brown solid.

LRMS: m/z 410(M+1)⁺

Retention time: 7.45 min (method B)

¹H NMR (300 MHz, CHLOROFORM-d) δ ppm 0.98 (t, J=7.28 Hz, 2H) 1.35-1.46 (m, 2H) 1.60-1.79 (m, 2H) 2.43 (s, 6H) 3.38 (t, J=7.14 Hz, 2H) 5.70 (br. s., 1H) 7.57 (s, 2H)

### Preparation 150

### 4-{5-[Butyl(2-fluorobenzoyl)amino]-1,3,4-thiadiazol-2-yl}-2,6-dimethylphenyl trifluoromethanesulfonate

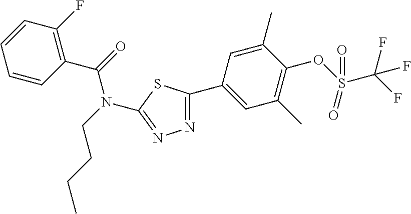

Obtained (37% yield) from the title compound of Preparation 149 and 2-fluorobenzoyl chloride following the procedure described in Preparation 85.

LRMS: m/z 532(M+1)⁺

Retention time: 7.93 min (method B)

¹H NMR (400 MHz, DMSO-d₆) δ ppm 0.68 (t, J=7.23 Hz, 3H) 1.01-1.21 (m, 2 H) 1.52-1.75 (m, 3H) 2.42 (s, 6H) 4.04 (t, 1H) 7.29-7.53 (m, 2H) 7.56-7.81 (m, 2H) 7.94 (s, 2H)

### Preparation 151

### N-Butyl-N-[5-(3,5-dimethyl-4-vinylphenyl)-1,3,4-thiadiazol-2-yl]-2-fluorobenzamide

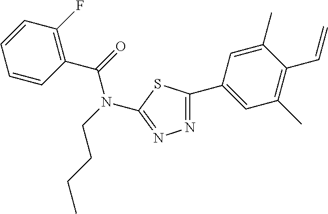

To a solution of the title compound of Preparation 150 (1.91 g, 3.59 mmol) and lithium chloride (0.50 g, 1.80 mmol) in dry DMF (40 mL) under nitrogen atmosphere, tributyl(vinyl)stannane (5.00 mL, 5.40 mmol) and tetrakis(triphenylphosphine) palladium (0.12 g, 0.10 mmol) were added. The reaction mixture was heated to 90° C. under nitrogen atmosphere and it was stirred at this temperature for 2 h. The mixture was cooled to room temperature, water and ethyl acetate were added and the aqueous layer was extracted with ethyl acetate (×2), the organic layers were washed with brine, dried, filtered and the solvent was evaporated un vacuo. The resulting crude was purified according to purification method A. 1.17 g (79% yield) of the title product were obtained.

LRMS: m/z 410(M+1)⁺

Retention time: 7.85 min (method B)

### Preparation 152

### N-Butyl-2-fluoro-N-[5-(4-formyl-3,5-dimethylphenyl)-1,3,4-thiadiazol-2-yl]benzamide

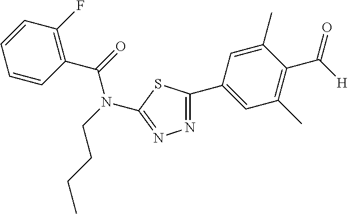

Obtained (98% yield) from the title compound of Preparation 151 following the procedure described in Preparation 78.

LRMS: m/z 412(M+1)⁺

Retention time: 7.45 min (method B)

### Preparation 153

### 4-(Allyloxy)-3,5-dimethylbenzonitrile

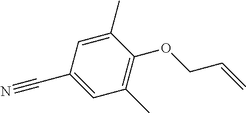

Obtained (69% yield) from 4-hydroxy-3,5-dimethylbenzonitrile and 3-bromoprop-1-ene following the procedure described in Preparation 20.

LRMS: m/z 188(M+1)⁺

Retention time: 6.53 min (method B)

¹H NMR (400 MHz, CHLOROFORM-d) δ ppm 2.30 (s, 6H) 4.34 (d, J=5.6 Hz, 2H), 5.43 (dd, J=17.1, 1.4 Hz, 2H), 6.08 (ddd, J=22.6, 10.8, 5.6 Hz, 1H), 7.33 (s, 2H)

### Preparation 154

### 5-[4-(Allyloxy)-3,5-dimethylphenyl]-N-butyl-1,3,4-thiadiazol-2-amine

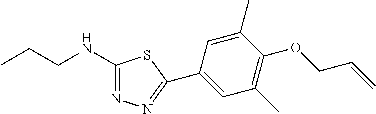

Obtained (17% yield) from the title compound of Preparation 1 and the title compound of Preparation 153 following the procedure described in Preparation 71.

LRMS: m/z 318(M+1)⁺

Retention time: 7.12 min (method B)

¹H NMR (200 MHz, CHLOROFORM-d) δ ppm 0.97 (t, 3H) 1.25-1.57 (m, 2H) 1.57-1.81 (m, 2H) 2.31 (s, 6H) 3.36 (t, J=7.03 Hz, 2H) 4.33 (dt, J=5.47, 1.37 Hz, 2H) 5.13-5.57 (m, 3H) 5.88-6.33 (m, 1H) 7.47 (s, 2H)

### Preparation 155

### N-{5-[4-(Allyloxy)-3,5-dimethylphenyl]-1,3,4-thiadiazol-2-yl}-N-butyl-2-methylbenzamide

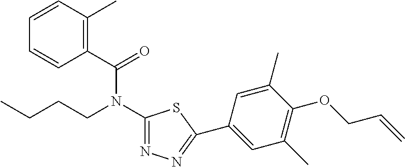

Obtained (94% yield) from the title compound of Preparation 154 and 2-methylbenzoyl chloride following the experimental procedure described in Preparation 85.

LRMS: m/z 436(M+1)⁺

Retention time: 7.87 min (method B)

### Preparation 156

### 3-Chloro-N-ethyl-2-fluoro-N-[5-(4-vinylphenyl)-1,3,4-thiadiazol-2-yl]benzamide

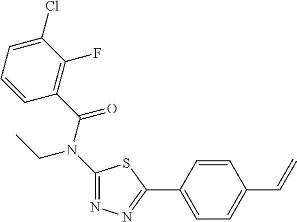

Obtained (8% yield) from the title compound of Preparation 84 and 3-chloro-2-fluorobenzoyl chloride following the experimental method described in Preparation 85.

LRMS: m/z 388(M+1)⁺

Retention time: 7.48 min (method B)

### Preparation 157

### 3-Chloro-N-ethyl-2-fluoro-N-[5-(4-formylphenyl)-1,3,4-thiadiazol-2-yl]benzamide

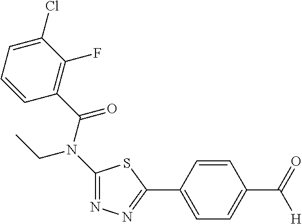

Obtained (35% yield) from the title compound of Preparation 156 following the procedure described for Preparation 78.

LRMS: m/z 390(M+1)⁺

Retention time: 6.95 min (method B)

### Preparation 158

### Benzyl 5-((4-(5-(butylamino)-1,3,4-thiadiazol-2-yl)-2,6-dimethylphenoxy)methyl)-2,2-dimethyloxazolidine-3-carboxylate

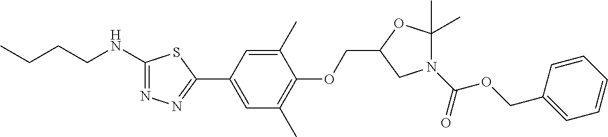

To a 0° C. cooled solution of the title compound of Preparation 19 (1.2 g, 4.33 mmol) in DMF (20 mL), sodium hydride (153 mg, 3.83 mmol) was added and the resulting mixture was stirred for 30 min at room temperature. Benzyl-2,2-dimethyl-5-((methylsulfonyloxy)methyl)oxazolidine-3-carboxylate (0.65 g, 1.91 mmol) was added and the mixture was stirred at 50° C. for 2 hours. Solvent was removed and crude purified following method A to yield 0.23 g (23%) of the title compound.

LRMS: m/z 425(M+1)⁺

Retention time: 7.57 min (method B)

### Preparation 159

### Benzyl 3-(4-(5-(N-butyl-2-fluorobenzamido)-1,3,4-thiadiazol-2-yl)-2,6-dimethylphenoxy)-2-hydroxypropylcarbamate

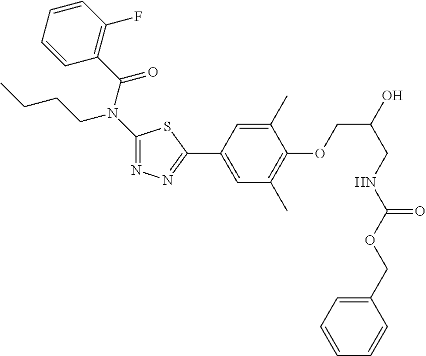

Obtained (44% yield) from the title compound of Preparation 158 and 2-fluorobenzoyl chloride following the procedure described for Example 105.

LRMS: m/z 607(M+1)⁺

Retention time: 7.40 min (method B)

### Preparation 160

### N-Butyl-3-chloro-2-fluoro-N-[5-(4-formyl-2-methylphenyl)-1,3,4-thiadiazol-2-yl]benzamide

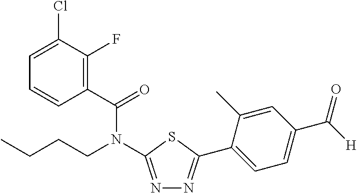

Obtained (88% yield) from tht title compound of Preparation 143 and 3-chloro-2-fluoro benzoic acid following the experimental procedure described in Preparation 81.

LRMS: m/z 432(M+1)⁺

Retention time: 7.45 min (method B)

### Preparation 161

### N-{5-[4-(Allyloxy)-3,5-dimethylphenyl]-1,3,4-thiadiazol-2-yl}-N-butyl-3-chloro-2-fluorobenzamide

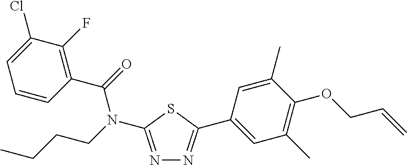

Obtained (72% yield) from the title compound of Preparation 154 and 3-chloro-2-fluorobenzoyl chloride following the procedure described in Preparation 85.

LRMS: m/z 474(M+1)⁺

Retention time: 7.95 min (method B)

### Preparation 162

### 3-Chloro-N-(cyclopropylmethyl)-2-fluoro-N-[5-(4-vinylphenyl)-1,3,4-thiadiazol-2-yl]benzamide

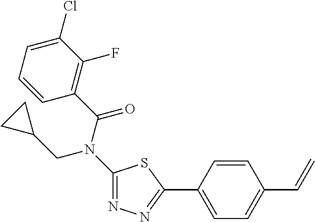

Obtained (85% yield) from the title compound of Preparation 97 and 3-chloro-2-fluorobenzoyl chloride following the procedure described in Preparation 85.

LRMS: m/z 414 (M+1)⁺

Retention time: 7.63 min (method B)

### Preparation 163

### 3-Chloro-N-(cyclopropylmethyl)-2-fluoro-N-[5-(4-formylphenyl)-1,3,4-thiadiazol-2-yl]benzamide

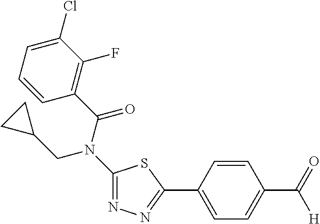

Obtained (92% yield) from the title compound of Preparation 162 following the procedure described in Preparation 78.

LRMS: m/z 416(M+1)⁺

Retention time: 7.63 min (method B)

### Preparation 164

### 4-{5-[Butyl(3-chloro-2-fluorobenzoyl)amino]-1,3,4-thiadiazol-2-yl}-2,6-dimethylphenyl trifluoromethanesulfonate

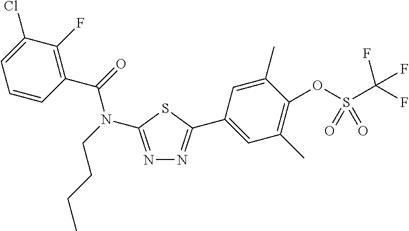

Obtained (34% yield) from the title compound of Preparation 149 and 3-chloro-2-fluorobenzoyl chloride following the procedure described in Preparation 85.

LRMS: m/z 566(M+1)⁺

Retention time: 8.05 min (method B)

### Preparation 165

### N-Butyl-3-chloro-N-[5-(3,5-dimethyl-4-vinylphenyl)-1,3,4-thiadiazol-2-yl]-2-fluorobenzamide

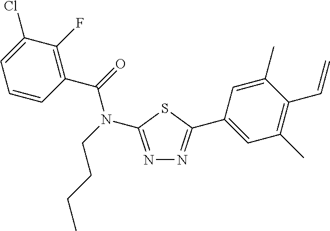

Obtained (85% yield) from the title compound of Preparation 164 following the procedure described in Preparation 151.

LRMS: m/z 444(M+1)⁺

Retention time: 8.03 min (method B)

### Preparation 166

### N-Butyl-3-chloro-2-fluoro-N-[5-(4-formyl-3,5-dimethylphenyl)-1,3,4-thiadiazol-2-yl]benzamide

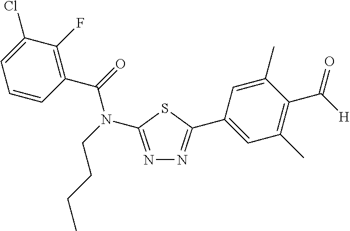

Obtained (41% yield) from the title compound of Preparation 165 following the procedure described in Preparation 78.

LRMS: m/z 446(M+1)⁺

Retention time: 7.68 min (method B)

### Preparation 167

### N-butyl-5-(3,5-dimethyl-4-vinylphenyl)-1,3,4-thiadiazol-2-amine

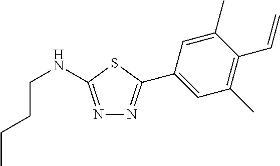

Obtained (84% yield) from the title compound of Preparation 149 following the procedure described in Preparation 151.

LRMS: m/z 288(M+1)⁺

Retention time: 7.28 min (method B)

### Preparation 168

### N-Butyl-2-chloro-N-[5-(3,5-dimethyl-4-vinylphenyl)-1,3,4-thiadiazol-2-yl]benzamide

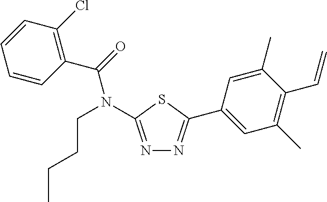

Obtained (76% yield) from the title compound of Preparation 167 and 2-chlorobenzoyl chloride following the procedure described in Preparation 85.

LRMS: m/z 426(M+1)⁺

Retention time: 7.95 min (method B)

### Preparation 169

### N-Butyl-2-chloro-N-[5-(4-formyl-3,5-dimethylphenyl)-1,3,4-thiadiazol-2-yl]benzamide

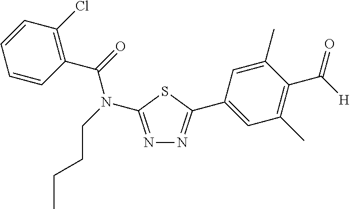

Obtained (100% yield) from the title compound of Preparation 168 following the procedure described in Preparation 78.

LRMS: m/z 428(M+1)⁺

Retention time: 7.60 min (method B)

¹H NMR (400 MHz, DMSO-d₆) δ ppm 0.70 (t, J=7.42 Hz, 3H) 1.07-1.23 (m, 2H) 1.68 (br. s., 2H) 2.65 (s, 6H) 3.60-4.40 (m, 2H) 7.52-7.67 (m, J=15.53, 7.67, 7.67, 5.86 Hz, 2H) 7.67-7.81 (m, 2H) 7.83 (s, 2H) 10.56 (s, 1H)

### EXAMPLES

### Example 1

### N-{5-[4-(aminosulfonyl)phenyl]-1,3,4-thiadiazol-2-yl}-N-butyl-3-methoxy-benzamide

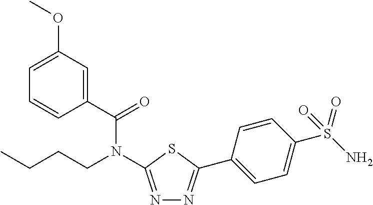

To a stirred solution of the title compound of preparation 4 (100 mg, 0.18 mmol) in dioxane (5 mL), 5 mL of HCl 4M in dioxane were added and the mixture was stirred at rt for 24 h. Solvent was removed in vacuo and resulting crude was purified following the purification method A to yield 40 mg (50% yield) of the title product.

LRMS: m/z 447 (M+1)⁺

Retention time: 16.34 (method C)

1H NMR (200 MHz, DMSO-d₆) δ ppm 0.71 (t, J=7.22 Hz, 3H) 0.99-1.27 (m, 2H) 1.67 (m, 2H) 3.80 (s, 3H) 4.09 (m, 2H) 7.19 (m, 4H) 7.48 (t, J=7.81 Hz, 2H) 7.96 (d, J=8.20 Hz, 2H) 8.19 (d, J=8.20 Hz, 2H)

### Example 2

### N-{5-[4-(aminosulfonyl)phenyl]-1,3,4-thiadiazol-2-yl}-N-butyl-2-naphthamide

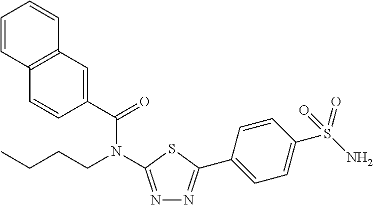

Obtained (43% yield) from the title compound of Preparation 5 following the procedure described in example 1.

LRMS: m/z 467 (M+1)⁺

Retention time: 17.90 min (method C)

¹H NMR (200 MHz, DMSO-d₆) δ ppm 0.67 (t, J=7.22 Hz, 3H) 1.14 (m, 2H) 1.73 (m, 2H) 4.22 (t, J=7.03 Hz, 2H) 7.28-7.86 (m, 5H) 7.86-8.53 (m, 8H).

### Example 3

### N-{5-[4-(aminosulfonyl)phenyl]-1,3,4-thiadiazol-2-yl}-N-butylbiphenyl-4-carboxamide

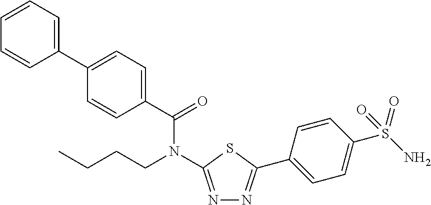

Obtained (41% yield) from the title compound of Preparation 6 following the procedure described in example 1.

LRMS: m/z 493 (M+1)⁺

Retention time: 18.78 min (method C)

¹H NMR (200 MHz, DMSO-d₆) δ ppm 0.75 (t, J=7.22 Hz, 3H) 0.95-1.39 (m, 2H) 1.53-1.91 (m, 2H) 4.21 (t, J=7.03 Hz, 2H) 7.26-7.63 (m, 5H) 7.64-8.09 (m, 8H) 8.22 (d, J=8.20 Hz, 2H).

### Example 4

### N-{5-[4-(aminosulfonyl)phenyl]-1,3,4-thiadiazol-2-yl}-4-butoxy-N-butylbenzamide

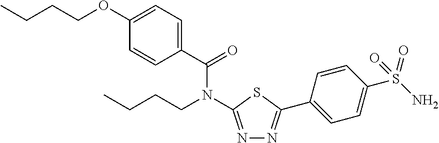

Obtained (14% yield) from the title compound of Preparation 7 following the procedure described in example 1.

LRMS: m/z 489 (M+1)⁺

Retention time: 19.24 min (method C)

¹H NMR (200 MHz, DMSO-d₆) δ ppm 0.75 (t, J=7.22 Hz, 3H) 0.95 (t, J=7.22 Hz, 3H) 1.25 (m, 2H) 1.45 (m, 2H) 1.65 (m, 4H) 4.07 (t, J=6.25 Hz, 2H) 4.20 (t, J=6.83 Hz, 2H) 7.10 (d, J=8.20 Hz, 2H) 7.51 (s, 2H) 7.62 (d, J=8.20 Hz, 2H) 7.97 (d, J=8.20 Hz, 2H) 8.19 (d, J=8.20 Hz, 2H).

### Example 5

### N-{5-[4-(aminosulfonyl)phenyl]-1,3,4-thiadiazol-2-yl}-N-butylbenzamide

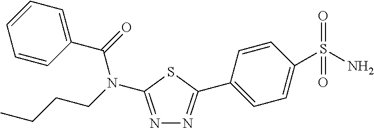

Obtained (90% yield) from the title compound of Preparation 8 following the procedure described in example 1.

LRMS: m/z 417 (M+1)⁺

Retention time: 16.02 min (method C)

¹H NMR (200 MHz, DMSO-d₆) δ ppm 0.71 (t, J=7.03 Hz, 3H) 1.16 (m, 2H) 1.69 (m, 2H) 4.13 (m, 2H) 7.63 (m, 5H) 7.98 (d, J=8.20 Hz, 2H) 8.21 (d, J=8.20 Hz, 2H) 8.44 (br. s., 2H)

### Example 6

### 3-(4-(5-(N-butyl-3-methoxybenzamido)-1,3,4-thiadiazol-2-yl)phenyl)propanoic acid

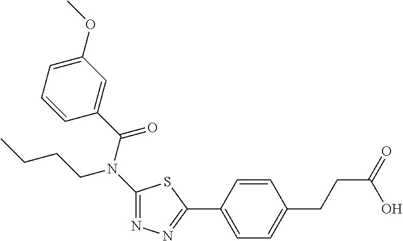

To a stirred solution of the title product of Preparation 14 (74 mg, 0.15 mmol) in dichloromethane (2 mL) at 0° C., 0.5 mL of trifluoroacetic acid were added and the mixture was stirred at rt overnight. Solvent was removed in vacuo and resulting crude was treated with diethyl ether. The solid thus obtained was filtered and washed with cold diethyl ether to yield 14 mg (21% yield) of the title product.

LRMS: m/z 440 (M+1)⁺

Retention time: 17.76 min (method C)

¹H NMR (200 MHz, DMSO-D6) δ ppm 0.7 (t, J=7.4 Hz, 3H) 1.1 (m, 2H) 1.7 (m, 2H) 2.6 (t, J=7.2 Hz, 2H) 2.9 (t, J=7.4 Hz, 2H) 3.8 (s, 3H) 4.1 (m, 2H) 7.2 (m, 3H) 7.5 (m, 3H) 7.9 (d, J=8.2 Hz, 2H) 12.2 (s, 1H)

### Example 7

### N-butyl-2-chloro-N-[5-(4-methoxy-3,5-dimethylphenyl)-1,3,4-thiadiazol-2-yl]benzamide

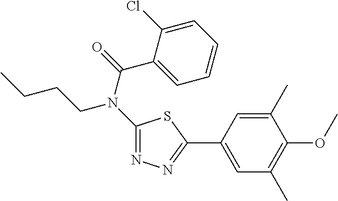

To a suspension of the title product of Preparation 9 (1 g, 3.43 mmol) in dichloromethane (40 mL), DIEA (2.4 mL, 13.72 mmol) was added and the mixture was stirred for 5 min. Then a solution of 2-chlorobenzoyl chloride (0.87 mL, 6.85 mmol) in dichloromethane (5 mL) was added dropwise and the final mixture was stirred overnight. The reaction mixture was diluted with dichloromethane and washed with HCl 2M, sat Na₂CO₃ solution and brine. The organic phase was dried on Na₂SO₄, filtered and the solvent was removed in vacuo to yield a solid that was purified according to purification method A. 620 mg of the title product were obtained (42% yield).

LRMS: m/z 430 (M+1)⁺

Retention time: 20.52 min (method C)

¹H NMR (200 MHz, DMSO-d₆) δ ppm 0.63 (t, J=7.20 Hz, 3H) 1.12 (m, 2H) 1.70 (m, 2H) 2.30 (s, 6H) 3.72 (s, 3H) 4.10 (m, 2H) 7.60-7.90 (m, 6H)

### Example 8

### N-butyl-2-chloro-N-[5-(4-hydroxy-3,5-dimethylphenyl)-1,3,4-thiadiazol-2-yl]benzamide

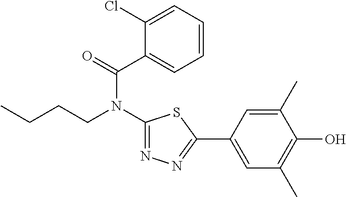

To a stirred suspension of the title product of example 7 (300 mg, 0.70 mmol) at −78° C. a solution of boron tribromide in dichloromethane 1M (1.40 mL, 1.40 mmol) was added and the mixture was stirred at that temperature for 15 min and the it was let to slowly reach rt during 2 h. Ice water was added to the reaction mixture and it was stirred at rt for 30 min. The organic layer was washed with brine, dried on Na₂SO₄ and solvent was removed in vacuo. The solid thus obtained was recrystallized from diisopropyl ether. 203 mg (70% yield) of the title product were obtained.

LRMS: m/z 416 (M+1)⁺

Retention time: 18.80 min (method C)

¹H NMR (200 MHz, DMSO-d₆) δ ppm 0.69 (t, J=7.22 Hz, 3H) 1.10 (m, 2H) 1.66 (m, 2H) 2.25 (s, 6H) 4.05 (m, 2H) 7.48-7.83 (m, 6H) 8.94 (br. s., 1H).

### Example 9

### N-butyl-2-fluoro-N-[5-(4-methoxy-3,5-dimethylphenyl)-1,3,4-thiadiazol-2-yl]benzamide

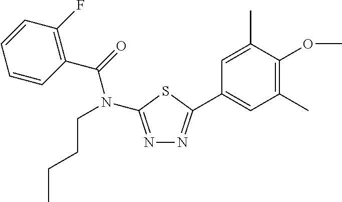

Obtained (22% yield) from 2-fluorobenzoyl chloride and the title compound of Preparation 9 following the procedure described in example 7.

LRMS: m/z 414 (M+1)⁺

Retention time: 19.94 min (method C)

¹H NMR (200 MHz, CDCl₃) δ ppm 0.8 (t, J=7.6 Hz, 3H) 1.2 (m, 2H) 1.7 (m, 2H) 2.4 (s, 6H) 3.8 (s, 3H) 4.1 (m, 2H) 7.3 (m, 2H) 7.5 (m, 2H) 7.7 (s, 2H).

### Example 10

### N-Butyl-2-fluoro-N-[5-(4-hydroxy-3,5-dimethylphenyl)-1,3,4-thiadiazol-2-yl]benzamide

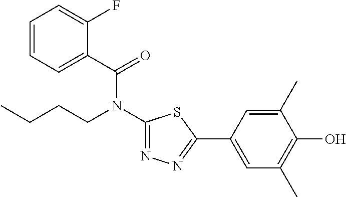

Obtained (14% yield) from the title compound of Example 9 following the procedure described in example 8.

LRMS: m/z 400 (M+1)⁺

Retention time: 18.00 min (method C)

¹H NMR (400 MHz, CDCl₃) δ ppm 0.77 (t, J=7.43 Hz, 3H) 1.09-1.32 (m, 2H) 1.74 (m, 2H) 2.32 (s, 6H) 4.14 (t, J=7.24 Hz, 2H) 7.03-7.60 (m, 4H) 7.70 (s, 2H).

### Example 11

### N-butyl-N-[5-(4-methoxy-3,5-dimethylphenyl)-1,3,4-thiadiazol-2-yl]benzamide

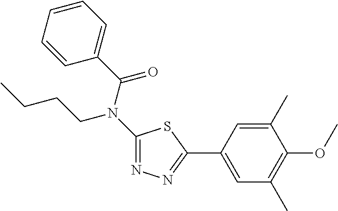

Obtained (27% yield) from benzoyl chloride and the title compound of Preparation 9 following the procedure described in example 7.

LRMS: m/z 396 (M+1)⁺

Retention time: 20.10 min (method C)

¹H NMR (200 MHz, DMSO-d₆) δ ppm 0.72 (t, J=7.26 Hz, 3H) 1.13 (sxt, J=7.26 Hz, 2H) 1.68 (quin, J=7.26 Hz, 2H) 2.31 (s, 6H) 3.78 (s, 3H) 4.09 (t, J=7.42 Hz, 2H) 7.58-7.93 (m, 7H).

### Example 12

### N-butyl-3-methoxy-N-[5-(4-methoxy-3,5-dimethylphenyl)-1,3,4-thiadiazol-2-yl]benzamide

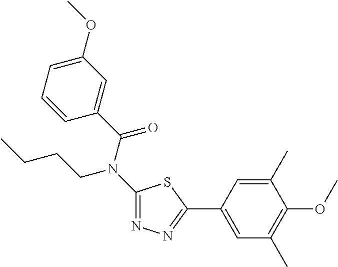

Obtained (31% yield) from 3-methoxybenzoyl chloride and the title compound of Preparation 9 following the procedure described in example 7.

LRMS: m/z 426 (M+1)⁺

Retention time: 20.15 min (method C)

¹H NMR (200 MHz, DMSO-D6) δ ppm 0.7 (t, J=7.4 Hz, 3H) 1.1 (m, 2H) 1.7 (m, 2H) 2.3 (s, 6H) 3.7 (s, 3H) 3.8 (s, 3H) 4.1 (m, 2H) 7.2 (m, 3H) 7.5 (t, J=8.0 Hz, 1H) 7.7 (s, 2H).

### Example 13

### N-butyl-N-[5-(4-methoxy-3,5-dimethylphenyl)-1,3,4-thiadiazol-2-yl]-1-naphthamide

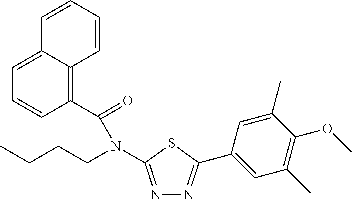

To a 0° C. stirred solution of the title compound of Preparation 9 (350 mg, 1.2 mmol) in THF (4 mL), triethylamine (0.52 mL, 3.6 eq), 1-naphthoyl chloride (360 mg, 1.44 mmol) and 4-DMAP (4 mg) were added. The final mixture was stirred at rt for 1 h and then at 60° C. overnight. It was let to cool down to rt and HCl 2M was added to pH 7. Solvent was removed in vacuo and the final crude was purified following method A. 348 mg (63% yield) of the title product were obtained.

LRMS: m/z 446 (M+1)⁺

Retention time: 21.00 min (method C)

¹H NMR (200 MHz, CDCl₃) δ ppm 0.62 (t, J=7.26 Hz, 3H) 1.05 (m, 2H) 2.37 (s, 6H) 3.79 (s, 3H) 7.42-7.84 (m, 7H) 7.98 (m, 2H)

### Example 14

N-butyl-2,6-dichloro-N-(5-(4-methoxy-3,5-dimethylphenyl)-1,3,4-thiadiazol-2-yl)benzamide

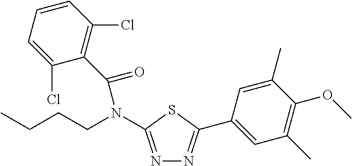

Obtained (31% yield) from 2,6-dichlorobenzoyl chloride and the title compound of Preparation 9 following the procedure described in example 13.

LRMS: m/z 464 (M+1)⁺

Retention time: 21.11 min (method C)

¹H NMR (200 MHz, CDCl₃) δ ppm 0.82 (t, J=7.22 Hz, 3H) 1.30 (m, 2H) 1.81 (m, 2H) 2.36 (s, 6H) 3.78 (s, 3H) 4.01 (m, 2H) 7.41 (m, 3H) 7.80 (s, 2H)

### Example 15

### N-butyl-N-[5-(4-methoxy-3,5-dimethylphenyl)-1,3,4-thiadiazol-2-yl]-2-(trifluoromethyl)benzamide

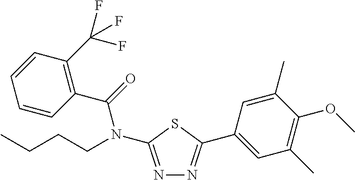

Obtained (45% yield) from 2-(trifluoromethyl)benzoyl chloride and the title compound of Preparation 9 following the procedure described in example 13.

LRMS: m/z 464 (M+1)⁺

Retention time: 20.40 min (method C)

¹H NMR (200 MHz, CDCl₃) 5 ppm 0.81 (t, J=7.22 Hz, 3H) 1.24 (m, 2H) 1.65-1.95 (m, 2H) 2.34 (s, 6H) 3.76 (s, 3H) 4.13 (m, 2H) 7.51-7.96 (m, 6H)

### Example 16

### N-butyl-N-[5-(4-methoxy-3,5-dimethylphenyl)-1,3,4-thiadiazol-2-yl]-2-phenylacetamide

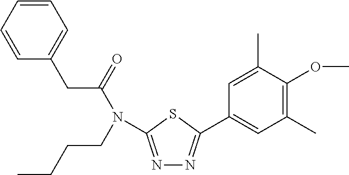

A microwave reactor containing a mixture of the title compound of preparation 9 (200 mg, 0.69 mmol), 2-phenylacetyl chloride (118 mg, 0.76 mmol), triethylamine (290 μl, 2.08 mol) and DMAP (10 mg, 0.08 mmol) in THF (2 ml) mixture was heated at a Biotage Initiator device at 90° C. and medium absorbance for 15 min in the microwave. Then 2N HCl was added and the solvent removed under reduced pressure. The crude obtained was purified according to purification method A to yield the desired product as a yellow solid (yield=29%)

LRMS: m/z 410 (M+1)⁺

Retention time: 20.14 min (method C)

¹H NMR (200 MHz, CDCl₃) δ ppm 0.98 (t, J=7.22 Hz, 3H) 1.45 (m, 2H) 1.80 (m, 2H) 2.30 (s, 6H) 3.76 (s, 3H) 4.05 (s, 2H) 4.28 (m, 2H) 7.30 (m, 5H) 7.60 (s, 2H).

### Example 17

### N-butyl-N-[5-(4-methoxy-3,5-dimethylphenyl)-1,3,4-thiadiazol-2-yl]-2-naphthamide

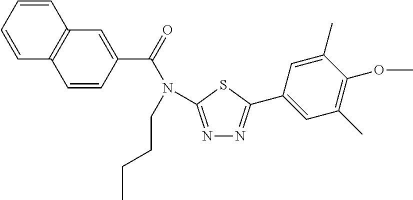

Obtained (3% yield) from 2-naphthoyl chloride and the title compound of Preparation 9 following the procedure described in example 16.

LRMS: m/z 446 (M+1)⁺

Retention time: 21.10 min (method C)

¹H NMR (200 MHz, CDCl₃) δ ppm 0.8 (t, J=7.2 Hz, 3H) 1.2 (m, 2H) 1.8 (m, 2 H) 2.4 (s, 6H) 3.8 (s, 3H) 4.3 (m, 2H) 7.6 (m, 3H) 7.7 (s, 2H) 8.0 (m, 4H).

### Example 18

### N-butyl-N-[5-(4-methoxy-3,5-dimethylphenyl)-1,3,4-thiadiazol-2-yl]-2,3-dihydro-1,4-benzodioxine-6-carboxamide

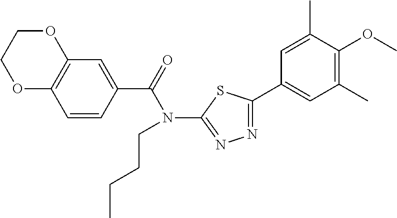

In a microwave oven vessel the title compound of Preparation 9 (250 mg, 0.86 mmol), 2,3-dihydrobenzo[b][1,4]dioxine-6-carboxylic acid (170 mg, 0.94 mmol), HBTU (325 mg, 0.86 mmol) and diisopropylethylamine (0.300 ml, 1.71 mmol) were placed and DMF (3 ml) was added. The mixture was heated at a Biotage Initiator device at 150° C. and high absorvance for 15 min. Then HCl 2M was added and the solvent removed in vacuo. The reaction crude was purified according to purification method A to yield 108 mg (27%) of the title compound.

LRMS: m/z 454 (M+1)⁺

Retention time: 19.90 min (method C)

¹H NMR (200 MHz, CDCl₃) δ ppm 0.80 (m, J=7.42 Hz, 3H) 1.28 (m, 2H) 1.79 (m, 2H) 2.35 (s, 6H) 3.77 (s, 3H) 4.32 (m, 6H) 6.88-7.17 (m, 2H) 7.23 (s, 2H) 7.66 (s, 1H).

### Example 19

### N-butyl-2-methoxy-N-[5-(4-methoxy-3,5-dimethylphenyl)-1,3,4-thiadiazol-2-yl]benzamide

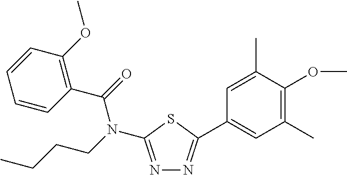

Obtained (27% yield) from 2-methoxybenzoyl chloride and the title compound of Preparation 9 following the procedure described in example 16.

LRMS: m/z 426 (M+1)⁺

Retention time: 19.60 min (method C)

¹H NMR (200 MHz, CDCl₃) δ ppm 0.8 (m, 3H) 1.2 (m, 2H) 1.7 (m, 2H) 2.3 (m, 6H) 3.8 (s, 3H) 3.9 (s, 3H) 4.2 (m, J=14.3, 6.4 Hz, 2H) 7.0 (d, J=8.6 Hz, 1H) 7.1 (d, J=7.8 Hz, 1H) 7.3 (m, 1H) 7.5 (m, 1H) 7.7 (s, 2H).

### Example 20

### N-Butyl-3-fluoro-N-[5-(4-methoxy-3,5-dimethylphenyl)-1,3,4-thiadiazol-2-yl]benzamide

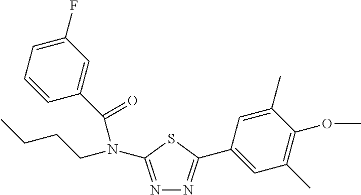

Obtained (36% yield) from the title compound of Preparation 9 and 3-fluorobenzoylchloride following the procedure described in example 16. Pyridine was used as solvent.

LRMS: m/z 414 (M+1)⁺

Retention time: 19.80 min (method C)

¹H NMR (400 MHz, CDCl₃) δ ppm 0.82 (t, J=7.24 Hz, 3H) 1.24 (m, 2H) 1.79 (m, 2H) 2.36 (s, 6H) 3.77 (s, 3H) 4.19 (m, 2H) 7.38 (m, 3H) 7.49 (m, 1H) 7.66 (s, 2H).

### Example 21

### N-Butyl-N-[5-(4-methoxy-3,5-dimethylphenyl)-1,3,4-thiadiazol-2-yl]nicotinamide

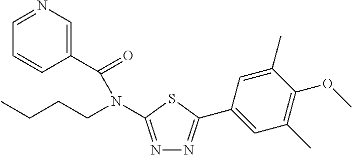

Obtained (34% yield) from the title compound of Preparation 9 and nicotinoyl chloride following the procedure described in example 16. Pyridine was used as solvent.

LRMS: m/z 397 (M+1)⁺

Retention time: 17.60 min (method C)

¹H NMR (200 MHz, CDCl₃) δ ppm 0.8 (t, J=7.4 Hz, 3H) 1.3 (m, 2H) 1.8 (m, 2H) 2.4 (s, 6H) 3.8 (s, 3H) 4.2 (m, 2H) 7.5 (dd, J=7.8, 5.9 Hz, 1H) 7.7 (s, 2H) 7.9 (m, 1H) 8.8 (m, 2H).

### Example 22

### N-Butyl-N-[5-(4-methoxy-3,5-dimethylphenyl)-1,3,4-thiadiazol-2-yl]pyridine-2-carboxamide

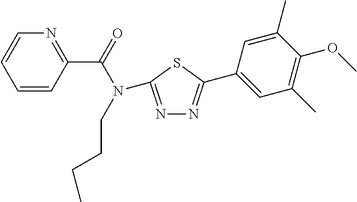

Obtained (21% yield) from the title compound of Preparation 9 and picolinoyl chloride following the procedure described in example 16. Pyridine was used as solvent.

LRMS: m/z 397 (M+1)⁺

Retention time: 19.00 min (method C)

¹H NMR (200 MHz, CDCl₃) δ ppm 0.83 (t, J=7.22 Hz, 13H) 1.26 (m, 2H) 1.87 (m, 2H) 2.36 (s, 6H) 3.77 (s, 3H) 4.41 (m, 2H) 7.49 (d, J=5.47 Hz, 1H) 7.67 (s, 2H) 7.74-8.00 (m, 2H) 8.69 (d, J=4.69 Hz, 1H)

### Example 23

### N-Butyl-6-fluoro-N-[5-(4-methoxy-3,5-dimethylphenyl)-1,3,4-thiadiazol-2-yl]pyridine-2-carboxamide

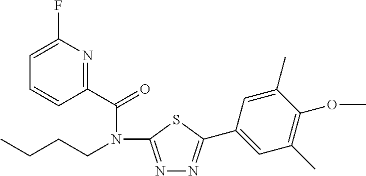

Obtained (5% yield) from the title compound of Preparation 9 and 6-fluoropicolinic acid following the procedure described in example 18.

LRMS: m/z 415 (M+1)⁺

Retention time: 19.70 min (method C)

¹H NMR (200 MHz, CDCl₃) δ ppm 0.87 (t, J=7.22 Hz, 3H) 1.31 (m, 2H) 1.93 (m, 2H) 2.35 (s, 6H) 3.77 (s, 3H) 4.37 (m, 2H) 7.00-7.40 (m, 2H) 7.45-8.25 (m, 3H)

### Example 24

### N-butyl-N-[5-(4-methoxy-3,5-dimethylphenyl)-1,3,4-thiadiazol-2-yl]-2-methylbenzamide

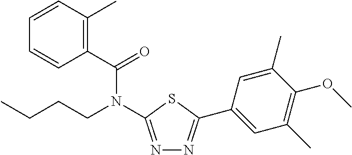

Obtained (30% yield) from the title compound of Preparation 9 and 2-methylbenzoyl chloride following the procedure described in example 16. Pyridine was used as solvent.

LRMS: m/z 410 (M+1)⁺

Retention time: 20.50 min (method C)

¹H NMR (200 MHz, CDCl₃) δ ppm 0.8 (t, J=7.2 Hz, 3H) 1.2 (m, 2H) 1.7 (m, 2 H) 2.3 (s, 6H) 2.4 (s, 3H) 3.8 (s, 3H) 4.1 (s, 2H) 7.3 (m, 4H) 7.7 (s, 2H).

### Example 25

### 2-chloro-N-[5-(4-methoxy-3,5-dimethylphenyl)-1,3,4-thiadiazol-2-yl]-Nmethylbenzamide

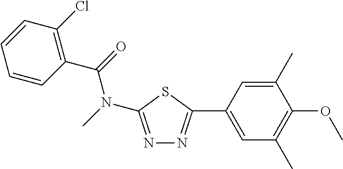

Obtained (60% yield) from the title compound of Preparation 15 and 2-chlorobenzoylchloride following the procedure described in example 13.

LRMS: m/z 388 (M+1)⁺

Retention time: 18.60 min (method C)

¹H NMR (200 MHz, CDCl₃) δ ppm 2.36 (s, 6H) 3.52 (s, 3H) 3.91 (s, 3H) 7.34-7.80 (m, 6H)

### Example 26

N-butyl-2-chloro-N-[5-(4-methoxy-3-methylphenyl)-1,3,4-thiadiazol-2-yl]benzamide

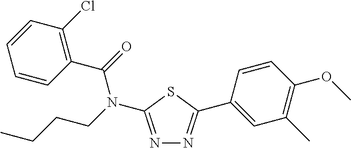

Obtained (44% yield) from the title compound of Preparation 16 and 2-chlorobenzoylchloride following the procedure described in example 13.

LRMS: m/z 416 (M+1)⁺

Retention time: 20.20 min (method C)

¹H NMR (200 MHz, DMSO-d₆) δ ppm 0.69 (t, J=7.22 Hz, 3H) 1.21 (m, 2H) 1.66 (m, 2H) 2.24 (s, 3H) 3.88 (s, 3H) 4.20 (m, 2H) 7.11 (d, J=8.20 Hz, 1H) 7.35-8.01 (m, 6H)

### Example 27

### N-butyl-2-chloro-N-[5-(3-chloro-4-methoxyphenyl)-1,3,4-thiadiazol-2-yl]benzamide

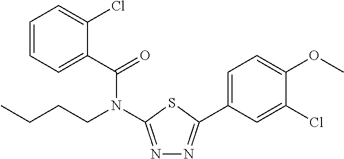

Obtained (49% yield) from the title compound of Preparation 17 and 2-chlorobenzoylchloride following the procedure described in example 13.

LRMS: m/z 437 (M+1)⁺

Retention time: 20.10 min (method C)

¹H NMR (200 MHz, DMSO-d₆) δ ppm 0.69 (t, J=7.42 Hz, 3H) 1.14 (m, 2H) 1.65 (m, 2H) 3.96 (s, 3H) 4.10 (m, 2H) 7.33 (d, J=8.98 Hz, 1H) 7.46-7.84 (m, 4H) 7.95 (dd, J=8.59, 1.95 Hz, 1H) 8.03 (s, 1H)

### Example 28

### Methyl 4-{5-[butyl(2-chlorobenzoyl)amino]-1,3,4-thiadiazol-2-yl}benzoate

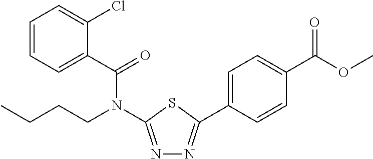

Obtained (58% yield) from the title compound of Preparation 18 and 2-chlorobenzoylchloride following the procedure described in example 13.

LRMS: m/z 430 (M+1)⁺

Retention time: 18.66 min (method C)

¹H NMR (200 MHz, DMSO-d₆) δ ppm 0.69 (t, J=7.22 Hz, 3H) 1.2 (m, 2H) 1.6 (m, 2H) 3.9 (s, 3H) 4.1 (m, 2H) 7.6 (m, 4H) 8.2 (m, 4H)

### Example 29

### 4-{5-[Butyl(2-chlorobenzoyl)amino]-1,3,4-thiadiazol-2-yl}benzoic acid

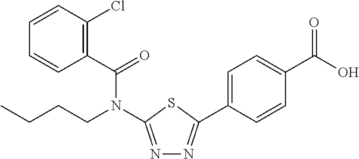

A suspension of the title compound of example 28 (200 mg, 0.47 mmol) in HCl conc (10 ml) was stirred at 50° C. overnight. The reaction mixture was cooled down and filtered. The solid thus obtained was washed with water and hexane and dried. 137 mg (71% yield) of the title product were obtained as a white solid.

LRMS: m/z 416 (M+1)⁺

Retention time: 18.00 min (method C)

¹H NMR (200 MHz, DMSO-d₆) δ ppm 0.70 (t, J=7.22 Hz, 3H) 1.16 (m, 2H) 1.68 (m, 2H) 3.93 (m, 2H) 7.47-7.86 (m, 4H) 8.01-8.27 (m, 4H)

### Example 30

### N-Butyl-2-chloro-N-{5-[4-(2,3-dihydroxypropoxy)-3,5-dimethylphenyl]-1,3,4-thiadiazol-2-yl}benzamide

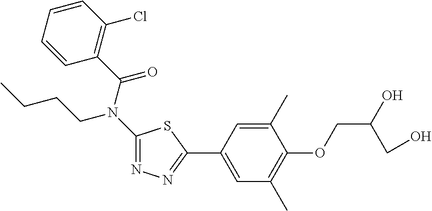

To a stirred solution of the title compound of Preparation 21 (56 mg, 0.11 mmol) in acetonitrile (2 ml), HCl 2M (0.1 ml, 0.20 mmol) was added and the mixture was stirred at 40° C. for 2 h. Solvent was removed and the resulting crude product was purified according to purification method A. 25 mg (47% yield) of the title compound were obtained.

LRMS: m/z 491 (M+1)⁺

Retention time: 17.40 min (method C)

¹H NMR (200 MHz, DMSO-d₆) δ ppm 0.69 (t, J=7.22 Hz, 3H) 1.12 (m, 2H) 1.65 (m, 2H) 2.33 (s, 6H) 3.45-3.96 (m, 5H) 4.66 (t, J=5.47 Hz, 1H) 4.98 (d, J=4.69 Hz, 1H) 7.40-7.94 (m, 6H).

### Example 31

### N-Butyl-2-chloro-N-[5-(2-chloro-6-methoxypyridin-4-yl)-1,3,4-thiadiazol-2-yl]benzamide

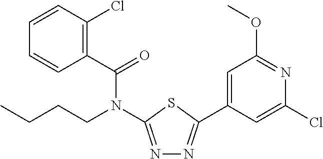

Obtained (58% yield) from the title compound of Preparation 22 and 2-chlorobenzoylchloride following the procedure described in example 13.

LRMS: m/z 438 (M+1)⁺

Retention time: 20.80 min (method C)

¹H NMR (200 MHz, DMSO-d₆) δ ppm 0.69 (t, J=7.42 Hz, 3H) 1.12 (m, 2H) 1.67 (m, 2H) 3.79 (m, 2H) 4.13 (s, 3H) 7.41 (s, 2H) 7.48-7.91 (m, 4H)

### Example 32

### N-butyl-2-chloro-N-{5-[4-(2-methoxyethoxy)-3,5-dimethylphenyl]-1,3,4-thiadiazol-2-yl}benzamide

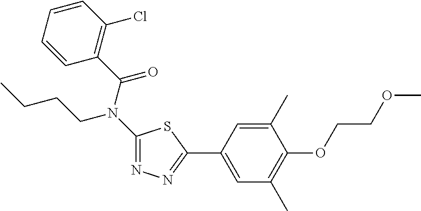

Obtained (21% yield) from the title compound of Preparation 26 and 2-chlorobenzoylchloride following the procedure described in example 13.

LRMS: m/z 475 (M+1)⁺

Retention time: 19.90 min (method C)

¹H NMR (200 MHz, CDCl₃) δ ppm 0.8 (t, J=7.4 Hz, 3H) 1.2 (m, 2H) 1.8 (m, 2 H) 2.4 (s, 6H) 3.5 (s, 3H) 3.8 (dd, J=6.1, 3.3 Hz, 2H) 4.0 (m, 2H) 4.3 (m, 2H) 7.5 (m, 4H) 7.7 (s, 2H).

### Example 33

### 2-Chloro-N-[5-(4-methoxy-3,5-dimethylphenyl)-1,3,4-thiadiazol-2-yl]-N-(2-methoxyethyl)benzamide

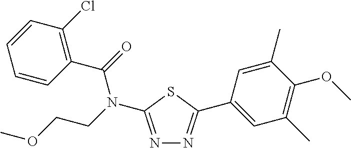

Obtained (16% yield) from the title compound of Preparation 24 and 2-chlorobenzoylchloride following the procedure described in example 16.

LRMS: m/z 432 (M+1)⁺

Retention time: 18.85 min (method C)

¹H NMR (200 MHz, CDCl₃) δ ppm 2.4 (s, 6H) 3.2 (s, 3H) 3.6 (m, 1H) 3.8 (s, 3 H) 3.9 (m, 1H) 4.1 (m, 1H) 4.5 (m, 1H) 7.4 (m, 4H) 7.7 (s, 2H).

### Example 34

### 2-Chloro-N-ethyl-N-[5-(4-methoxy-3,5-dimethylphenyl)-1,3,4-thiadiazol-2-yl]benzamide

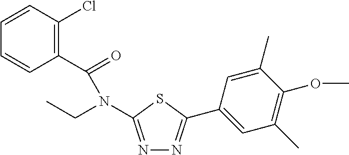

Obtained (20% yield) from the title compound of Preparation 25 and 2-chlorobenzoylchloride following the procedure described in example 16.

LRMS: m/z 402 (M+1)⁺

Retention time: 19.00 min (method C)

¹H NMR (200 MHz, CDCl₃) δ ppm 1.32 (t, J=7.03 Hz, 3H) 2.36 (s, 6H) 3.78 (s, 3H) 7.45 (m, 4H) 7.67 (s, 2H)

### Example 35

### tert-Butyl (4-{5-[butyl(2-chlorobenzoyl)amino]-1,3,4-thiadiazol-2-yl}-2,6-dimethyl-phenoxy)acetate

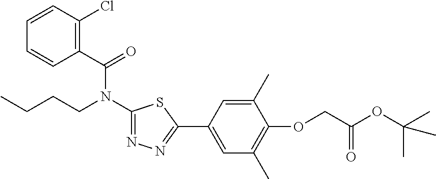

Obtained (70% yield) from the title compound of Preparation 34 and 2-chlorobenzoylchloride following the procedure described in example 13.

LRMS: m/z 530 (M+1)⁺

Retention time: 21.01 min (method C)

¹H NMR (200 MHz, DMSO-D6) δ ppm 0.7 (t, J=7.4 Hz, 3H) 1.2 (m, 2H) 1.5 (s, 9H) 1.7 (m, 2H) 2.3 (s, 6H) 3.8 (m, 1H) 4.1 (m, 1H) 4.5 (s, 2H) 7.7 (m, 6H).

### Example 36

### (4-{5-[Butyl(2-chlorobenzoyl)amino]-1,3,4-thiadiazol-2-yl}-2,6-dimethylphenoxy)acetic acid

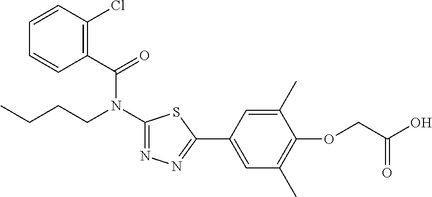

Obtained (45% yield) from the title compound of Example 35 following the procedure described in example 6.

LRMS: m/z 474 (M+1)⁺

Retention time: 18.31 min (method C)

¹H NMR (200 MHz, DMSO-D6) δ ppm 0.7 (t, J=7.22 Hz, 3H) 1.2 (m, 2H) 1.7 (m, 2H) 2.33 (s, 6H) 3.60 (t, J=6.25 Hz, 2H) 4.47 (s, 2H) 7.7 (m, 6H).

### Example 37

### 2-Chloro-N-[5-(4-methoxy-3,5-dimethylphenyl)-1,3,4-thiadiazol-2-yl]-N-(3-methylbutyl)benzamide

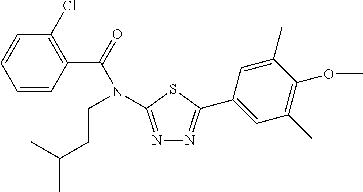

Obtained (30% yield) from the title compound of Preparation 28 and 2-chlorobenzoylchloride following the procedure described in example 16.

LRMS: m/z 444 (M+1)⁺

Retention time: 20.90 min (method C)

¹H NMR (200 MHz, CDCl₃) δ ppm 0.74 (s, 6H) 1.50 (m, 2H) 2.36 (s, 6H) 3.78 (s, 3H) 4.12 (m, 2H) 7.72 (m, 6H)

### Example 38

### 2-Chloro-N-[3-(diethylamino)propyl]-N-[5-(4-methoxy-3,5-dimethylphenyl)-1,3,4-thiadiazol-2-yl]benzamide

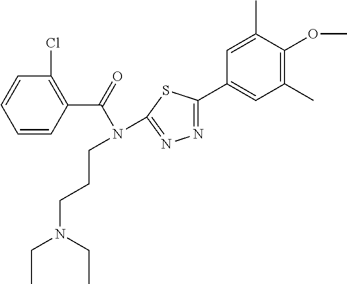

Obtained (30% yield) from the title compound of Preparation 30 and 2-chlorobenzoylchloride following the procedure described in example 16.

LRMS: m/z 488 (M+1)⁺

Retention time: 13.70 min (method C)

¹H NMR (200 MHz, CDCl₃) δ ppm 0.9 (t, J=7.2 Hz, 6H) 2.4 (m, 12H) 3.8 (s, 3H) 3.9 (m, 1H) 4.3 (m, 1H) 7.5 (m, 4H) 7.7 (s, 2H).

### Example 39

### N-Butyl-2-chloro-N-[5-(2-chloro-6-methylpyridin-4-yl)-1,3,4-thiadiazol-2-yl]benzamide

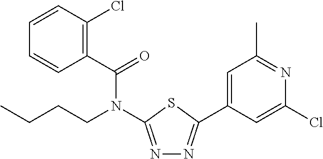

Obtained (25% yield) from the title compound of Preparation 31 and 2-chlorobenzoylchloride following the procedure described in example 13.

LRMS: m/z 422 (M+1)⁺

Retention time: 19.80 min (method C)

¹H NMR (200 MHz, DMSO-d₆) δ ppm 0.69 (t, J=7.22 Hz, 3H) 1.15 (m, 2H) 1.65 (m, 2H) 2.51 (s, 3H) 3.95-4.24 (m, 2H) 7.42-7.80 (m, 4H) 7.95 (s, 2H)

### Example 40

### N-Butyl-2-chloro-N-[5-(6-chloro-2-oxo-1,2-dihydropyridin-4-yl)-1,3,4-thiadiazol-2-yl]benzamide

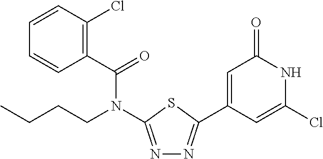

To a stirred solution of the title compound of Example 31 (100 mg, 0.23 mmol) in acetonitrile (5 ml) under argon, sodium iodide (172 mg, 1.15 mmol) and trimethylsilyl chloride (145 μl, 1.15 mmol) were added. The mixture was stirred at R.T. for 1 h and then water (50 ml) was added. The aqueous solution was extracted with dichloromethane, then basified and extracted with dichloromethane again. The combined organic layers were dried and solvent was removed. The crude product thus obtained was purified according to purification method A to yield 82 mg (85%) of the title compound.

LRMS: m/z 424 (M+1)⁺

Retention time: 18.40 min (method C)

¹H NMR (200 MHz, DMSO-d₆) δ ppm 0.69 (t, J=7.26 Hz, 3H) 1.14 (sxt, J=7.26 Hz, 2H) 1.67 (m, 2H) 3.85-4.18 (m, 2H) 7.18 (s, 2H) 7.45-7.88 (m, 4H).

### Example 41

### N-Butyl-N-[5-(2-chloro-6-methoxypyridin-4-yl)-1,3,4-thiadiazol-2-yl]-2-fluorobenzamide

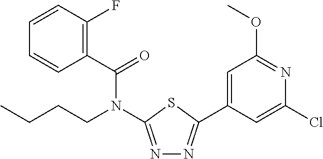

Obtained (68% yield) from the title compound of Preparation 22 and 2-fluorobenzoylchloride following the procedure described in example 13.

LRMS: m/z 421 (M+1)⁺

Retention time: 20.40 min (method C)

¹H NMR (200 MHz, CDCl₃) δ ppm 0.87 (t, J=7.22 Hz, 3H) 1.31 (m, 2H) 1.93 (m, 2H) 3.77 (s, 3H) 4.37 (m, 2H) 7.42 (m, 3H) 7.75 (m, 3H).

### Example 42

### N-Butyl-N-[5-(2-chloro-6-methylpyridin-4-yl)-1,3,4-thiadiazol-2-yl]-2-fluorobenzamide

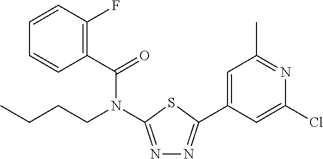

Obtained (40% yield) from the title compound of Preparation 31 and 2-fluorobenzoylchloride following the procedure described in example 13.

LRMS: m/z 405 (M+1)⁺

Retention time: 19.20 min (method C)

¹H NMR (200 MHz, DMSO-d₆) δ ppm 0.70 (t, J=7.22 Hz, 3H) 1.10 (m, 2H) 1.65 (m, 2H) 2.53 (s, 3H) 4.09 (t, J=7.22 Hz, 2H) 7.42 (m, 2H), 7.68 (m, 2H) 7.92 (s, 2H)

### Example 43

### 2-fluoro-N-[5-(4-methoxy-3,5-dimethylphenyl)-1,3,4-thiadiazol-2-yl]-N-propylbenzamide

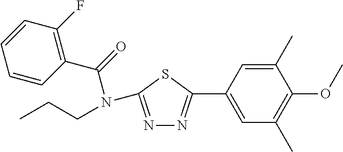

Obtained (40% yield) from the title compound of Preparation 33 and 2-fluorobenzoylchloride following the procedure described in example 16.

LRMS: m/z 400 (M+1)⁺

Retention time: 19.30 min (method C)

¹H NMR (200 MHz, CDCl₃) δ ppm 0.80 (t, J=7.42 Hz, 3H) 1.78 (m, 2H) 2.36 (s, 6H) 3.78 (s, 3H) 4.08 (m, 2H) 7.23 (m, 2H) 7.45 (m, 2H) 7.66 (s, 2H).

### Example 44

### N-Butyl-2-fluoro-N-[5-(2-methylpyridin-4-yl)-1,3,4-thiadiazol-2-yl]benzamide

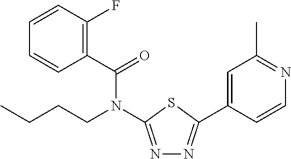

To a stirred solution of title compound of Example 42 (60 mg, 0.15 mmol) in ethanol (10 ml), 10% Pd/C (12 mg) and HCl in dioxane (0.1 ml) were added and the mixture was stirred under hydrogen (30 psi) for 4 days. The reaction mixture was filtered and solvent removed in vacuo to yield a crude product that was purified according to purification method A to yield 36 mg (65%) of the title compound.

LRMS: m/z 371 (M+1)⁺

Retention time: 15.90 min (method C)

¹H NMR (200 MHz, DMSO-d₆) δ ppm 0.70 (t, J=7.42 Hz, 3H) 1.05 (m, 2H) 1.64 (m, 2H) 2.54 (s, 3H) 4.09 (t, J=7.42 Hz, 2H) 7.40 (m, 2H) 7.64 (m, 2H) 7.82 (s, 2H) 8.63 (d, J=5.08 Hz, 1H)

### Example 45

### N-butyl-2-fluoro-N-[5-(2-methoxypyridin-4-yl)-1,3,4-thiadiazol-2-yl]benzamide

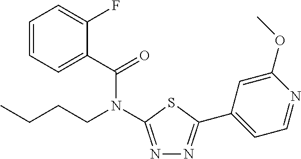

Obtained (16% yield) from the title compound of Preparation 36 and 2-fluorobenzoylchloride following the procedure described in example 13.

LRMS: m/z 387 (M+1)⁺

Retention time: 18.70 min (method C)

¹H NMR (200 MHz, DMSO-d₆) δ ppm 0.70 (t, J=7.22 Hz, 3H) 1.16 (m, 2H) 1.66 (m, 2H) 3.85 (s, 3H) 4.09 (m, 2H) 7.37-7.80 (m, 6H) 8.35 (d, J=5.08 Hz, 1H).

### Example 46

### N-(cyclopropylmethyl)-2-fluoro-N-[5-(4-methoxy-3,5-dimethylphenyl)-1,3,4-thiadiazol-2-yl]benzamide

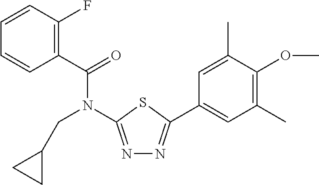

Obtained (10% yield) from the title compound of Preparation 38 and 2-fluorobenzoylchloride following the procedure described in example 16.

LRMS: m/z 419 (M+1)⁺

Retention time: 19.44 min (method C)

¹H NMR (200 MHz, CDCl₃) δ ppm 0.1 (q, J=5.3 Hz, 2H) 0.4 (m, 2H) 1.3 (m, 1 H) 2.4 (s, 6H) 3.8 (s, 3H) 4.1 (d, J=7.0 Hz, 2H) 7.3 (m, 2H) 7.5 (m, 2H) 7.7 (s, 2H).

### Example 47

### 3-(4-{5-[butyl(2-fluorobenzoyl)amino]-1,3,4-thiadiazol-2-yl}-2,6-dimethylphenyl)propanoic acid

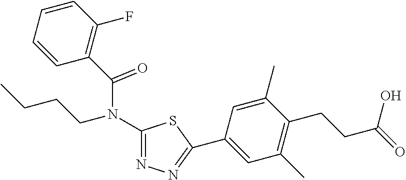

Obtained (6% yield) from the title compound of Preparation 59 following the procedure described in example 1.

LRMS: m/z 456 (M+1)⁺

Retention time: 18.01 min (method C)

### Example 48

### N-butyl-2-fluoro-N-(5-(6-methoxypyridin-3-yl)-1,3,4-thiadiazol-2-yl)benzamide

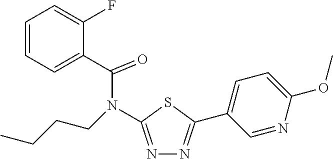

Obtained (20% yield) from the title compound of Preparation 40 and 2-fluorobenzoylchloride following the procedure described in example 13.

LRMS: m/z 387 (M+1)⁺

Retention time: 18.26 min (method C)

¹H NMR (200 MHz, CDCl₃) δ ppm 0.77 (t, J=8 Hz, 3H) 1.23 (m, 2H) 1.74 (m, 2 H) 4.01 (s, 3H) 4.16 (m, 2H) 6.87 (d, J=10 Hz, 1H) 7.35 (m, 2H) 7.52 (m, 2 H) 8.23 (m, 1H) 8.73 (s, 1H).

### Example 49

### N-butyl-2-fluoro-N-(5-(imidazo[1,2-a]pyridin-6-yl)-1,3,4-thiadiazol-2-yl)benzamide

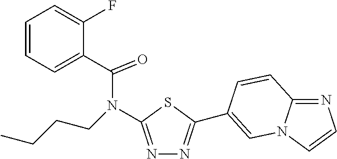

Obtained (36% yield) from the title compound of Preparation 41 and 2-fluorobenzoylchloride following the procedure described in example 13.

LRMS: m/z 396 (M+1)⁺

Retention time: 11.96 min (method C)

¹H NMR (200 MHz, DMSO-d₆) δ ppm 0.71 (t, J=8 Hz, 3H) 1.17 (m, 2H) 1.67 (m, 2H) 4.08 (t, J=8 Hz, 2H) 7.46 (m, 2H) 7.75 (m, 4H) 8.07 (s, 1H) 8.36 (s, 1H) 9.41 (s, 1H).

### Example 50

### N-butyl-2-fluoro-N-(5-(imidazo[1,2-a]pyridin-7-yl)-1,3,4-thiadiazol-2-yl)benzamide

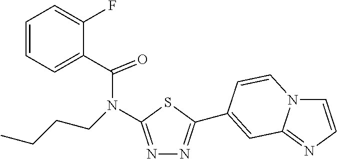

Obtained (34% yield) from the title compound of Preparation 44 and 2-fluorobenzoylchloride following the procedure described in example 13.

LRMS: m/z 396 (M+1)⁺

Retention time: 11.91 min (method C)

¹H NMR (200 MHz, DMSO-d₆) δ ppm 0.71 (t, J=8 Hz, 3H) 1.17 (m, 2H) 1.67 (m, 2H) 4.08 (t, J=6 Hz, 2H) 7.46 (m, 2H) 7.57 (m, 2H) 7.74 (m, 2H) 8.15 (d, J=16 Hz, 2H) 8.70 (d, J=6 Hz, 1H).

### Example 51

### 3-(4-(5-(N-butyl-2-chlorobenzamido)-1,3,4-thiadiazol-2-yl)benzamido)propanoic acid

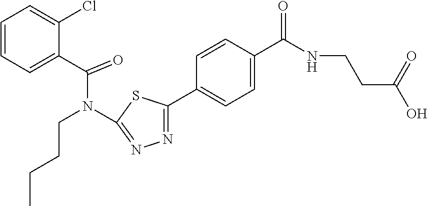

A mixture of the title compound of Example 29 (50 mg, 0.12 mmol), EDC (25 mg, 0.13 mmol) and HOBt (18 mg, 0.13 mmol) in THF (2 ml) was stirred at room temperature for 2 h. Then ethyl 3-aminopropanoate (16 mg, 0.14 mmol) was added and the reaction mixture stirred at rt overnight. Solvent was removed and the crude redissolved in ethyl acetate. The organic layer was washed with water, dried and solvent was removed. TFA (1 ml) and DCM (1 ml) were added and the final mixture was stirred at rt overnight. Solvent was removed in vacuo and the oil obtained purified according to purification method A. 15 mg of the title compound were obtained (yield=49%) as a white solid.

LRMS: m/z 487 (M+1)⁺

Retention time: 16.67 min (method C)

¹H NMR (200 MHz, CDCl₃) δ ppm 0.76 (t, J=7.03 Hz, 3H) 1.20 (m, 2H) 1.48-2.01 (m, 2H) 2.57-2.89 (m, 2H) 3.54-3.96 (m, 2H) 4.27 (d, J=8.20 Hz, 2H) 7.47 (m, 4H) 7.76-8.23 (m, 4H).

### Example 52

### Ethyl 3-(4-(5-(N-butyl-2-chlorobenzamido)-1,3,4-thiadiazol-2-yl)benzamido)propanoate

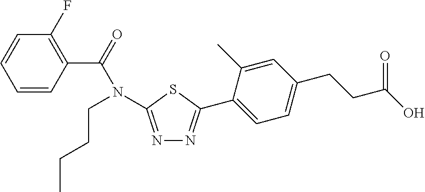

Obtained (2% yield) from the title compound of Preparation 51 following the procedure described in example 29.

LRMS: m/z 442 (M+1)⁺

Retention time: 18.14 min (method C)

¹H NMR (400 MHz, CHLOROFORM-d) δ ppm 0.77 (s, 3H) 1.10-1.33 (m, 2H) 1.64-1.83 (m, 2H) 2.70-2.80 (m, 2H) 2.93-3.08 (m, 2H) 4.09-4.22 (m, 2H) 7.13-7.24 (m, 2H) 7.24-7.25 (m, 1H) 7.29-7.34 (m, 1H) 7.43-7.49 (m, 1H) 7.50-7.58 (m, 1H) 7.64-7.68 (m, 1H).

### Example 53

### N-butyl-N-(5-(4-carbamoylphenyl)-1,3,4-thiadiazol-2-yl)-2-chlorobenzamide

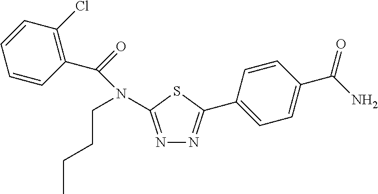

Obtained (23% yield) from the title compound of Example 29 and ammonia following the procedure described in example 51.

LRMS: m/z 415 (M+1)⁺

Retention time: 16.51 min (method C)

¹H NMR (200 MHz, DMSO-d₆) δ ppm 0.70 (t, J=7.03 Hz, 3H) 1.01-1.25 (m, 2 H) 1.65 (d, J=8.59 Hz, 2H) 3.86 (br. s., 2H) 7.43-7.84 (m, 4H) 7.93-8.22 (m, 4H) 8.39 (s, 2H).

### Example 54

### 1-(4-(5-(N-butyl-2-fluorobenzamido)-1,3,4-thiadiazol-2-yl)benzyl)azetidine-3-carboxylic acid

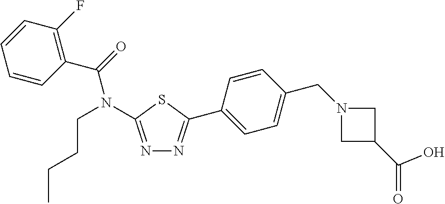

Obtained (23% yield) from the title compound of Preparation 47 and azetidine-3-carboxylic acid following the procedure described in Preparation 10.

LRMS: m/z 469 (M+1)⁺

Retention time: 13.02 min (method C)

¹H NMR (200 MHz, DMSO-d₆) δ ppm 0.72 (t, 3H) 1.16 (m, 2H) 1.66 (m, 2H) 3.24 (m., 1H) 3.34-3.53 (m, 2H) 3.64 (s, 6H) 4.06 (t, 2H) 7.46 (m, 4H) 7.61-7.86 (m, 2H) 7.95 (m, 2H).

### Example 55

### (R)—N-butyl-N-(5-(4-(2,3-dihydroxypropoxy)-3,5-dimethylphenyl)-1,3,4-thiadiazol-2-yl)-2-fluorobenzamide

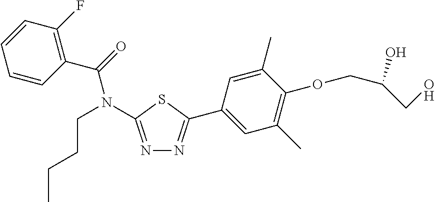

Obtained (16% yield) from the title compound of Preparation 61 following the procedure described in Example 30.

LRMS: m/z 475 (M+1)⁺

Retention time: 16.96 min (method C)

¹H NMR (200 MHz, CDCl₃) δ ppm 0.73 (t, J=7.22 Hz, 3H) 1.18 (m, 2H) 2.33 (s, 6H) 3.86 (m, 4H) 4.66 (m, 3H) 7.40-7.84 (m, 6H).

### Example 56

### 2-fluoro-N-(5-(4-methoxy-3,5-dimethylphenyl)-1,3,4-thiadiazol-2-yl)-N-phenethylbenzamide

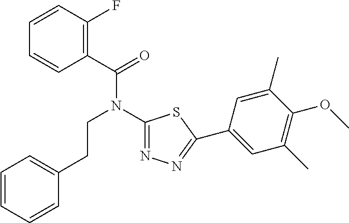

Obtained (27% yield) from the title compound of Preparation 52 and 2-fluorobenzoylchloride following the procedure described in example 13.

LRMS: m/z 462 (M+1)⁺

Retention time: 20.26 min (method C)

¹H NMR (200 MHz, CDCl₃) δ ppm 2.4 (s, 6H) 3.1 (t, J=7.0 Hz, 2H) 3.8 (s, 3H) 4.4 (m, 2H) 7.0 (m, 3H) 7.2 (m, 5H) 7.5 (m, 1H) 7.7 (s, 2H).

### Example 57

### 2-(4-(5-(N-butyl-2-fluorobenzamido)-1,3,4-thiadiazol-2-yl)benzamido)acetic acid

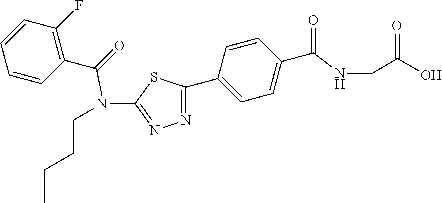

Obtained (5% yield) from the title compound of Preparation 63 and tert-butyl 2-aminoacetate following the procedure described in example 51

LRMS: m/z 457 (M+1)⁺

Retention time: 15.85 min (method C)

¹H NMR (200 MHz, CDCl₃) δ ppm 0.6 (t, J=7.0 Hz, 3H) 1.1 (m, 2H) 1.6 (m, 2H) 4.0 (m, 4H) 7.2 (m, 2H) 7.4 (m, 2H) 7.8 (m, 4H) 8.2 (s, 1H).

### Example 58

### N-Butyl-2-fluoro-N-[5-(1-methyl-2-oxo-1,2-dihydropyridin-4-yl)-1,3,4-thiadiazol-2-yl]benzamide

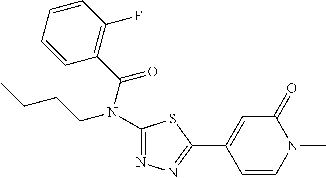

To a stirred suspension of title compound of Preparation 67 (114 mg, 0.43 mmol) in THF (3 mL), DMAP (10 mg, 0.08 mmol), DIEA (0.23 mL, 1.32 mmol) and 2-fluorobenzoyl chloride (82 mg, 0.52 mmol) were added. The resulting suspension was stirred under nitrogen atmosphere at room temperature overnight. The reaction mixture was diluted with dichloromethane, washed with water and brine, dried, filtered and evaporated under vacuum to give a crude which was purified using a Biotage 40+S column (5% methanol in dichloromethane) and then crystallized using diethyl ether to yield 55 mg of the title compound (49% yield) as a white crystalline solid.

LRMS: m/z 387 (M+1)⁺

Retention time: 15.41 min (method C)

¹H NMR (200 MHz, CHLOROFORM-d) δ ppm 0.77 (t, J=7.22 Hz, 3H) 1.04-1.38 (m, 2H) 1.63-1.84 (m, 2H) 3.60 (s, 3H) 4.07-4.27 (m, 2H) 6.96 (dd, J=7.03, 1.95 Hz, 1H) 7.05 (d, J=1.95 Hz, 1H) 7.15-7.67 (m, 5H)

### Example 59

### N-(4-{5-[Butyl(2-fluorobenzoyl)amino]-1,3,4-thiadiazol-2-yl}benzyl)-beta-alanine

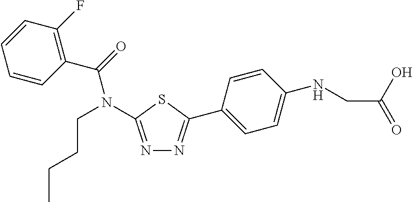

Obtained (28% yield) from the title compound of Preparation 47 and β-alanine following the procedure described in Preparation 10.

LRMS: m/z 457 (M+1)⁺

Retention time: 12.32 min (method C)

¹H NMR (200 MHz, DMSO-d6) δ ppm 0.70 (t, J=10.45 Hz, 3H) 1.06-1.23 (m, 2H) 1.64 (m, 2H) 2.35 (t, J=6.64 Hz, 3H) 2.76 (t, J=6.64 Hz, 2H) 3.83 (s, 2H) 4.01-4.12 (m, 2H) 7.37-7.56 (m, 4H) 7.58-7.81 (m, 3H) 7.96 (d, J=8.20 Hz, 2H).

### Example 60

### N-Butyl-2-fluoro-N-[5-(1-methyl-6-oxo-1,6-dihydropyridin-3-yl)-1,3,4-thiadiazol-2-yl]benzamide

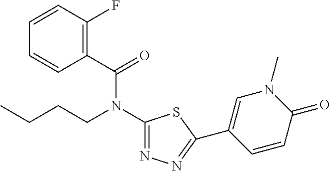

Obtained (10% yield) from the title compound of Preparation 69 and 2-fluorobenzoyl chloride following the procedure described in Example 58.

LRMS: m/z 387 (M+1)⁺

Retention time: 15.35 min (method C)

¹H NMR (200 MHz, CHLOROFORM-d) δ ppm 0.67-0.86 (m, 3H) 1.03-1.36 (m, 2H) 1.73 (quin, J=7.51 Hz, 2H) 3.65 (s, 3H) 4.13 (d, J=7.51 Hz, 2H) 6.69 (d, J=9.37 Hz, 1H) 7.16-7.37 (m, 2H) 7.39-7.62 (m, 2H) 7.92 (dd, J=9.57, 2.54 Hz, 1H) 8.08 (d, J=2.54 Hz, 1H).

### Example 61

### N-Butyl-2-fluoro-N-[5-(6-oxo-1,6-dihydropyridin-3-yl)-1,3,4-thiadiazol-2-yl]benzamide

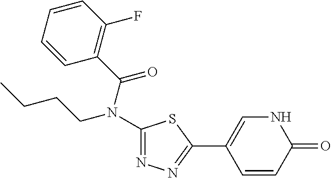

To a stirred solution of the title compound of Example 48 (84 mg, 0.16 mmol) in acetonitrile (3 mL), sodium iodide (160 mg, 1.08 mmol) and trimethylsilyl chloride (0.14 mL, 1.11 mmol) were added and the mixture was stirred at room temperature for 1.5 h. The reaction mixture was poured into ice/water, extracted with dichloromethane and the organic layer washed with water, brine, dried, filtered and the solvent removed in vacuo to yield a crude product which was purified according to purification method A to yield 21 mg (36%) of the title compound.

LRMS: m/z 373 (M+1)⁺

Retention time: 14.54 min (method C)

¹H NMR (200 MHz, CHLOROFORM-d) δ ppm 0.69 (t, J=7.22 Hz, 3H) 1.02-1.22 (m, 2H) 1.53-1.73 (m, 2H) 3.57 (s, 1H) 3.95-4.12 (m, 2H) 6.50 (d, J=9.37 Hz, 1H) 7.36-7.53 (m, 2H) 7.59-7.78 (m, 2H) 7.97-8.14 (m, 2H)

### Example 62

### N-Butyl-2-fluoro-N-[5-(6-oxo-1,6-dihydropyridin-3-yl)-1,3,4-thiadiazol-2-yl]benzamide

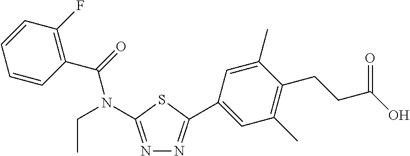

Obtained (28% yield) from the title compound of Preparation 74 following the procedure described in Example 6.

LRMS: m/z 428 (M+1)+

Retention time: 19.20 min (method C)

¹H NMR (200 MHz, DMSO-d₆) δ ppm 1.45 (t, 3H) 2.37 (s, 6H) 2.78-2.95 (m, 2H) 4.50 (q, 2H) 7.21-7.38 (m, 2H) 7.55 (s, 2H) 8.02-8.22 (m, 1H) 8.31-8.49 (m, 1H)

### Example 63

### N-Butyl-N-[5-(4-{[(2S)-2,3-dihydroxypropyl]oxy}-3,5-dimethylphenyl)-1,3,4-thiadiazol-2-yl]-2-fluorobenzamide

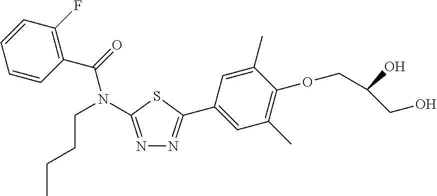

Obtained (96% yield) from the title compound of Preparation 75 following the procedure described in Example 30.

LRMS: m/z 474 (M+1)⁺

Retention time: 16.93 min (method C)

¹H NMR (200 MHz, CHLOROFORM-d) δ ppm 0.72-0.83 (m, 3H) 1.24 (q, J=7.29 Hz, 2H) 1.72 (d, J=7.42 Hz, 2H) 2.36 (s, 6H) 3.75-3.97 (m, 4H) 4.04-4.22 (m, 3H) 7.13-7.37 (m, 2H) 7.48 (dd, J=11.91, 6.05 Hz, 2H) 7.67 (s, 2H)

### Example 64

### 1-(4-{5-[butyl(2-fluorobenzoyl)amino]-1,3,4-thiadiazol-2-yl}benzyl)piperidine-4-carboxylic acid

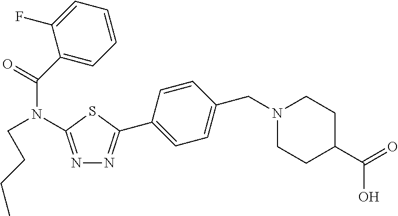

Obtained (10% yield) from the title compound of Preparation 47 and piperidine-4-carboxylic acid following the procedure described in Example 54.

LRMS: m/z 497 (M+1)⁺

Retention time: 12.12 min (method C)

¹H NMR (200 MHz, DMSO-d6) δ ppm 0.60-0.83 (m, 3H) 0.99-1.26 (m, 2H) 1.60 (m, 6H) 2.15 (m, 3H) 2.66-2.89 (m, 2H) 3.51 (s, 2H) 4.03 (d, J=8.20 Hz, 2H) 7.33-7.54 (m, 4H) 7.58-7.81 (m, 2H) 7.93 (d, J=8.20 Hz, 2H)

### Example 65

### 1-(4-{5-[Butyl(2-methoxybenzoyl)amino]-1,3,4-thiadiazol-2-yl}benzyl)azetidine-3-carboxylic acid

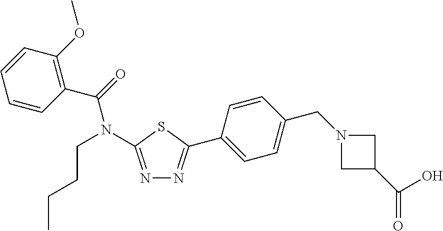

Obtained (82% yield) from the title compound of Preparation 78 and azetidine-3-carboxylic acid following the procedure described in Example 54.

LRMS: m/z 481 (M+1)⁺

Retention time: 13.02 min (method C)

¹H NMR (200 MHz, DMSO-d6) δ ppm 0.68 (t, 3H) 0.99-1.23 (m, 2H) 1.52-1.72 (m, 2H) 3.32-3.49 (m, 5H) 3.62 (s, 5H) 4.01-4.16 (m, 2H) 7.04-7.28 (m, 2H) 7.37-7.62 (m, 4H) 7.92 (d, J=8.59 Hz, 2H)

### Example 66

### N-(Cyclopropylmethyl)-N-[5-(4-{[(2R)-2,3-dihydroxypropyl]oxy}-3,5-dimethyl-phenyl)-1,3,4-thiadiazol-2-yl]-2-fluorobenzamide

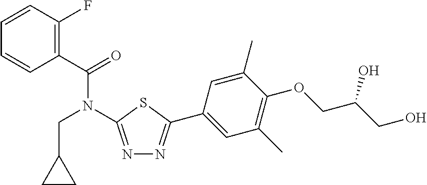

To a stirred solution of the title compound of Preparation 80 (360 mg, 0.92 mmol) in THF (4 ml) at 0° C., NaH 60% (75 mg, 1.88 mmol) was added in one portion and mixture stirred for 30 min. A solution of 2-fluorobenzoyl chloride (295 mg, 1.86 mmol) in THF (3 ml) was then added dropwise to this mixture and stirred at room temperature for 1 h. To this solution, HCl 2M (3.7 ml, 7.4 mmol) was added and the mixture was stirred at rt for 1 h and then at 60° C. for 1 h. Mixture was partitioned between water and dichloromethane, the organic layer was dried and solvent removed to yield a crude product that was purified according to purification method A. 140 mg (51% yield) of the title compound were obtained.

LRMS: m/z 472 (M+1)⁺

Retention time: 16.13 min (method C)

¹H NMR (200 MHz, CHLOROFORM-d) δ ppm 0.13 (m, J=5.34 Hz, 2H) 0.37-0.52 (m, 2H) 1.15-1.37 (m, 1H) 2.37 (s, 6H) 3.75-3.89 (m, 2H) 3.93 (d, J=5.08 Hz, 2H) 4.14 (m, J=4.49 Hz, 3H) 7.13-7.37 (m, 2H) 7.44-7.58 (m, 2 H) 7.67 (s, 2H)

### Example 67

### 1-(4-{5-[butyl(pyridin-3-ylcarbonyl)amino]-1,3,4-thiadiazol-2-yl}benzyl)azetidine-3-carboxylic acid

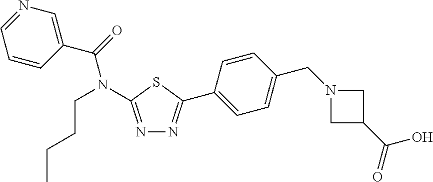

Obtained (7% yield) from the title compound of Preparation 82 and azetidine-3-carboxylic acid following the procedure described in Example 54.

LRMS: m/z 452 (M+1)⁺

Retention time: 10.04 min (method C)

¹H NMR (200 MHz, DMSO-d6) δ ppm 0.67-0.81 (m, 3H) 1.06-1.26 (m, 2H) 1.59-1.82 (m, 2H) 3.16-3.25 (m, 3H) 3.32-3.52 (m, 2H) 3.62 (s, 2H) 3.99-4.17 (m, 2H) 7.43 (s, 1H) 7.47 (s, 1H) 7.62 (dd, J=8.00, 4.88 Hz, 1H) 7.92 (s, 1H) 7.96 (s, 1H) 8.10-8.22 (m, 1H) 8.80 (dd, J=5.08, 1.17 Hz, 1H) 8.89 (d, J=2.34 Hz, 1H).

### Example 68

### 1-(4-{5-[Ethyl(2-fluorobenzoyl)amino]-1,3,4-thiadiazol-2-yl}benzyl)azetidine-3-carboxylic acid

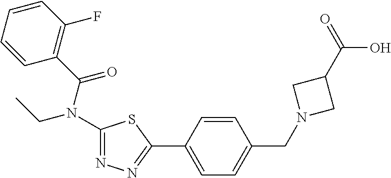

Obtained (44% yield) from the title compound of Preparation 86 and azetidine-3-carboxylic acid following the procedure described in Example 54.

LRMS: m/z 441 (M+1)⁺

Retention time: 11.13 min (method C)

¹H NMR (200 MHz, DMSO-d₆) δ ppm 0.72 (t, J=7.22 Hz, 3H) 3.09-3.50 (m, 5 H) 3.59-3.70 (m, 2H) 4.01 (d, J=9.37 Hz, 2H) 7.47 (m, 4H) 7.60-7.80 (m, 2 H) 7.96 (d, 2H)

### Example 69

### N-(4-{5-[Butyl(2-fluorobenzoyl)amino]-1,3,4-thiadiazol-2-yl}benzoyl)-beta-alanine

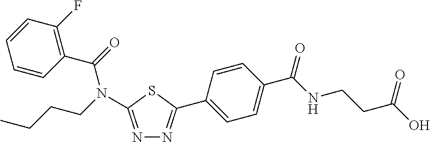

Obtained (76% yield) from the title compound of Preparation 87 following the same procedure described in Example 6.

LRMS: m/z 471 (M+1)⁺

Retention time: 15.94 min (method C)

¹H NMR (200 MHz, DMSO-d6) δ ppm 0.71 (t, 3H) 1.02-1.28 (m, 2H) 1.57-1.80 (m, 2H) 2.42-2.60 (m, 2H) 3.41-3.55 (m, 2H) 4.01-4.18 (m, 2H) 7.39-7.54 (m, 2H) 7.62-7.81 (m, 2H) 7.96-8.05 (m, 2H) 8.07-8.15 (m, 2H) 8.77 (t, J=5.66 Hz, 1H).

### Example 70

### N-(Cyclopropylmethyl)-N-[5-(4-{[(2S)-2,3-dihydroxypropyl]oxy}-3,5-dimethyl-phenyl)-1,3,4-thiadiazol-2-yl]-2-fluorobenzamide

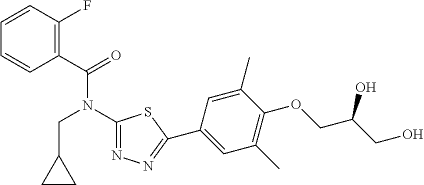

Obtained (50% yield) from the title compound of Preparation 88 and 2-fluorobenzyl chloride following the procedure described in Example 66.

LRMS: m/z 472 (M+1)⁺

Retention time: 16.31 min (method C)

¹H NMR (200 MHz, CHLOROFORM-d) δ ppm 0.07-0.20 (m, 2H) 0.44 (q, J=5.99 Hz, 2H) 1.16-1.36 (m, 1H) 2.36 (s, 6H) 3.86 (t, J=4.49 Hz, 2H) 3.93 (d, J=5.08 Hz, 2H) 4.05-4.21 (m, 3H) 7.13-7.37 (m, 2H) 7.44-7.61 (m, 2H) 7.67 (s, 2H)

### Example 71

### 1-(4-{5-[(2-Fluorobenzoyl)(propyl)amino]-1,3,4-thiadiazol-2-yl}benzyl)azetidine-3-carboxylic acid

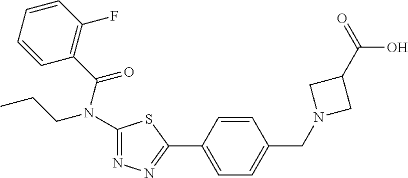

Obtained (81% yield) from the title compound of Preparation 91 and azetidine-3-carboxylic acid following the same procedure described in Example 54.

LRMS: m/z 455 (M+1)⁺

Retention time: 12.19 min (method C)

¹H NMR (200 MHz, DMSO-d₆) δ ppm 0.72 (t, J=7.22 Hz, 3H) 1.57-1.79 (m, 2H) 3.11-3.52 (m, 5H) 3.62-3.72 (m, 2H) 4.04 (d, J=9.37 Hz, 2H) 7.43 (d, J=3.12 Hz, 4H) 7.62-7.84 (m, 2H) 7.97 (d, 2H)

### Example 72

### (3R)-3-[(4-{5-[Butyl(2-fluorobenzoyl)amino]-1,3,4-thiadiazol-2-yl}benzoyl)amino]-butanoic acid

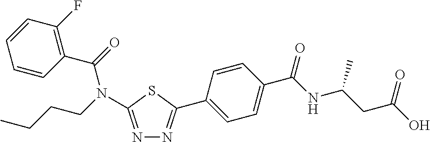

Obtained (69% yield) from the title compound of Preparation 92 following the same procedure described in Example 62.

LRMS: m/z 485 (M+1)⁺

Retention time: 16.43 min (method C)

¹H NMR (200 MHz, DMSO-d6) δ ppm 0.70 (t, J=7.22 Hz, 3H) 1.02-1.38 (m+d, 2H+3H) 1.56-1.80 (m, 2H) 2.31-2.70 (m, 2H) 3.96-4.19 (t, 2H) 4.21-4.53 (m, 1H) 7.31-7.58 (m, 2H) 7.62-7.86 (m, 2H) 7.87-8.20 (m, 4H) 8.58 (d, 1H)

### Example 73

### (3S)-3-[(4-{5-[butyl(2-fluorobenzoyl)amino]-1,3,4-thiadiazol-2-yl}benzoyl)amino]-butanoic acid

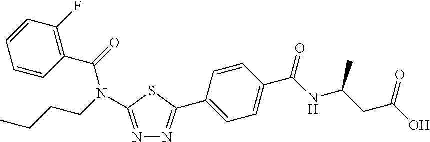

Obtained (93% yield) from the title compound of Preparation 93 following the same procedure described in Example 62.

LRMS: m/z 485 (M+1)⁺

Retention time: 16.43 min (method C)

¹H NMR (200 MHz, CHLOROFORM-d) δ ppm 0.77 (t, 3H) 1.09-1.32 (m, 2H) 1.39 (d, 3H) 1.63-1.85 (m, 2H) 2.75 (t, J=4.49 Hz, 2H) 4.11-4.27 (m, 2H) 4.52-4.73 (m, 1H) 7.05 (d, J=8.98 Hz, 1H) 7.17-7.38 (m, 2H) 7.41-7.63 (m, 2H) 7.83-7.95 (d, 2H) 8.07 (d, J=8.59 Hz, 2H)

### Example 74

### N-{5-[4-(Aminomethyl)phenyl]-1,3,4-thiadiazol-2-yl}-N-butyl-2-fluorobenzamide

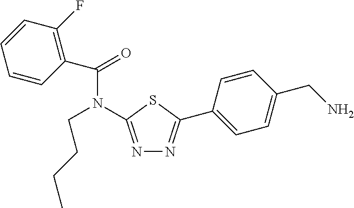

To a solution of the title compound of Preparation 47 (1.00 g, 2.61 mmol) in methanol (40 mL), hydroxylamine (0.39 g, 5.61 mmol) in water (2 mL) was added and the solution was stirred at room temperature for 2 hours. Zinc (0.37 g, 5.70 mmol) was added and the resulting suspension was stirred at room temperature overnight. The reaction mixture was filtered through Celite, washed with methanol and the solvent evaporated under vacuum. The resulting crude was purified according to purification method A and the resulting solid was dissolved in HCl 4N in dioxane (5 mL), stirred for 5 hours, concentrated and crystallized with diethyl ether to yield 220 mg (21%) of the title compound as hydrochloride salt.

LRMS: m/z 385 (M+1)⁺

Retention time: 10.75 min (method C)

¹H NMR (200 MHz, DMSO-d6) δ ppm 0.70 (t, 3H) 1.04-1.26 (m, 2H) 1.55-1.76 (m, 2H) 4.00-4.15 (m, 4H) 7.35-7.54 (m, 2H) 7.60-7.81 (m, 4H) 8.06 (d, J=8.20 Hz, 2H)

### Example 75

### 1-[2-(4-{5-[butyl(2-fluorobenzoyl)amino]-1,3,4-thiadiazol-2-yl}phenyl)ethyl]azetidine-3-carboxylic acid

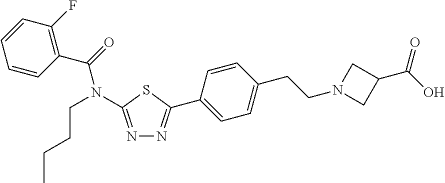

Obtained (11% yield) from the title compound of Preparation 96 following the same procedure described in Example 54.

LRMS: m/z 483 (M+1)⁺

Retention time: 13.54 min (method C)

### Example 76

### 1-(4-{5-[(cyclopropylmethyl)(2-fluorobenzoyl)amino]-1,3,4-thiadiazol-2-yl}benzyl)azetidine-3-carboxylic acid

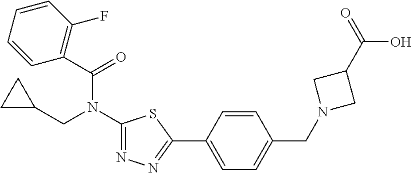

Obtained (21% yield) from the title compound of Preparation 99 and azetidine-3-carboxylic acid following the same procedure described for the synthesis of Example 54.

LRMS: m/z 467 (M+1)⁺

Retention time: 12.10 min (method C)

¹H NMR (200 MHz, DMSO-d₆) δ 0.04 (q, J=4.82 Hz, 2H) 0.26-0.47 (m, 2H) 0.94-1.31 (m, 2H) 3.18 (d, J=1.95 Hz, 4H) 3.37 (br. s., 2H) 3.57 (s, 2H) 3.98 (d, J=6.64 Hz, 2H) 7.38 (dd, J=8.00, 2.15 Hz, 3H) 7.52-7.79 (m, 3H) 7.88 (d, J=8.20 Hz, 2H)

### Example 77

### 1-(4-{5-[(2-fluorobenzoyl)(3-methylbutyl)amino]-1,3,4-thiadiazol-2-yl}benzyl)-azetidine-3-carboxylic acid

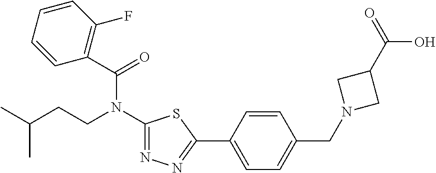

Obtained (16% yield) from the title compound of Preparation 102 and azetidine-3-carboxylic acid following the procedure described in Example 54.

LRMS: m/z 483 (M+1)⁺

Retention time: 14.03 min (method C)

¹H NMR (200 MHz, DMSO-d₆) δ ppm 0.68 (d, J=6.64 Hz, 6H) 1.30-1.67 (m, 3 H) 3.12-3.73 (m, 9H) 4.05 (d, J=7.81 Hz, 2H) 7.45 (d, J=8.20 Hz, 3H) 7.74 (t, J=7.22 Hz, 3H) 7.95 (d, J=8.20 Hz, 2H)

### Example 78

### 1-(4-{5-[(2-fluorobenzoyl)(methyl)amino]-1,3,4-thiadiazol-2-yl}benzyl)azetidine-3-carboxylic acid

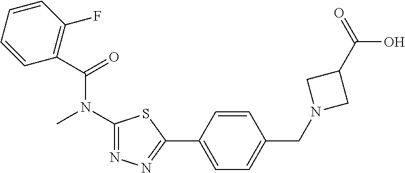

Obtained (5% yield) from the title compound of Preparation 105 and azetidine-3-carboxylic acid following the procedure described in Example 54.

LRMS: m/z 427 (M+1)⁺

Retention time: 10.16 min (method C)

¹H NMR (200 MHz, DMSO-d₆) δ ppm 2.97-3.83 (m, 10H) 7.45 (d, J=7.42 Hz, 4H) 7.58-7.81 (m, 2H) 7.94 (d, J=7.81 Hz, 2H)

### Example 79

### 1-(4-{5-[butyl(2-fluorobenzoyl)amino]-1,3,4-thiadiazol-2-yl}-3-methylbenzyl)-azetidine-3-carboxylic acid

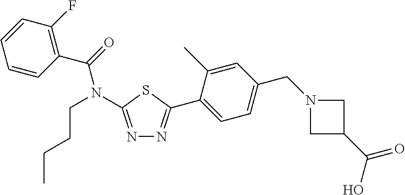

Obtained (27% yield) from the title compound of Preparation 111 and azetidine-3-carboxylic acid following the procedure described in Example 54.

LRMS: m/z 483(M+1)⁺

Retention time: 13.55 min (method C)

¹H NMR (200 MHz, DMSO-d₆) δ ppm 0.70 (t, J=7.22 Hz, 3H) 1.02-1.27 (m, J=7.32, 7.32, 7.32, 7.32, 7.03 Hz, 2H) 1.67 (quin, J=7.32 Hz, 2H) 3.22 (br. s., 3H) 3.43 (br. s., 3H) 3.49-3.65 (m, 4H) 4.07 (t, J=7.61 Hz, 2H) 7.26 (d, J=8.20 Hz, 1H) 7.36-7.58 (m, 2H) 7.67 (d, J=7.81 Hz, 2H)

### Example 80

### 4-[(4-{5-[butyl(2-fluorobenzoyl)amino]-1,3,4-thiadiazol-2-yl}benzoyl)amino]-butanoic acid

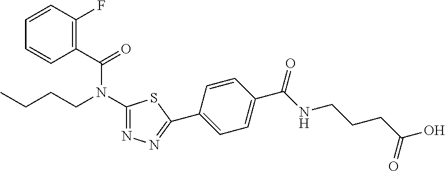

Obtained (76% yield) from the title compound of Preparation 112 following the same procedure described for the synthesis of Example 62.

LRMS: m/z 485(M+1)⁺

Retention time: 16.18 min (method C)

¹H NMR (200 MHz, CHLOROFORM-d) δ ppm 0.77 (t, J=7.22 Hz, 3H) 1.07-1.33 (m, 2H) 1.73 (s, 2H) 1.90-2.13 (m, 2H) 2.53 (t, J=6.44 Hz, 2H) 3.57 (q, J=5.86 Hz, 2H) 4.17 (t, 2H) 6.81-6.94 (m, 1H) 7.14-7.38 (m, 2H) 7.40-7.63 (m, 2H) 7.90 (d, 2H) 8.04 (d, 2H)

### Example 81

### 1-(4-{5-[(2-fluorobenzoyl)(2-methoxyethyl)amino]-1,3,4-thiadiazol-2-yl}benzyl)-azetidine-3-carboxylic acid

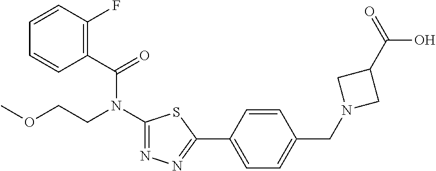

Obtained (28% yield) from the title compound of Preparation 115 and azetidine-3-carboxylic acid following the procedure described in Example 54.

LRMS: m/z 471(M+1)⁺

Retention time: 10.78 min (method C)

¹H NMR (300 MHz, DMSO-d₆) δ ppm 3.49-3.70 (m, 11H) 4.20-4.38 (m, 2H) 7.33-7.51 (m, 4H) 7.60-7.78 (m, 2H) 7.94 (d, J=8.24 Hz, 2H)

### Example 82

### 1-(4-{5-[butyl(phenylacetyl)amino]-1,3,4-thiadiazol-2-yl}benzyl)azetidine-3-carboxylic acid

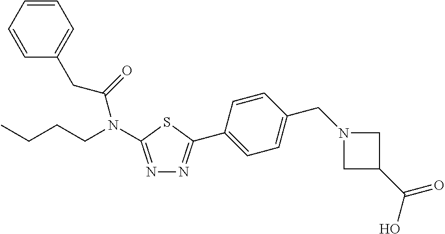

Obtained (7% yield) from the title compound of Preparation 116 and azetidine-3-carboxylic acid following the procedure described for the synthesis of Example 54.

LRMS: m/z 465(M+1)⁺

Retention time: 13.49 min (method C)

¹H NMR (200 MHz, DMSO-d6) δ ppm 0.93 (t, 3H) 1.28-1.52 (m, 2H) 1.69 (q, 2H) 3.31-3.50 (m, 5H) 3.63 (s, 2H) 4.20 (s, 2H) 4.30 (t, 2H) 7.28-7.47 (m, 7H) 7.86 (d, J=8.20 Hz, 2H)

### Example 83

### 1-(4-{5-[butyl(2,6-difluorobenzoyl)amino]-1,3,4-thiadiazol-2-yl}benzyl)azetidine-3-carboxylic acid

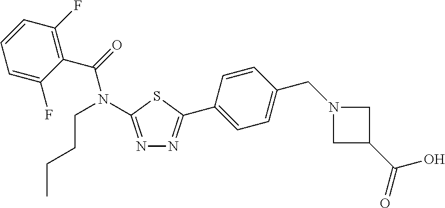

Obtained (9% yield) from the title compound of Preparation 117 and azetidine-3-carboxylic acid following the procedure described in Example 54.

LRMS: m/z 487(M+1)⁺

Retention time: 11.96 min (method C)

### Example 84

### N-(4-{5-[butyl(phenylacetyl)amino]-1,3,4-thiadiazol-2-yl}benzyl)-beta-alanine

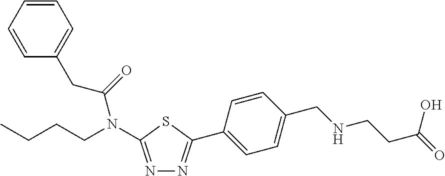

Obtained (41% yield) from the title compound of Preparation 116 and β-alanine following the procedure described for the synthesis of Example 54.

LRMS: m/z 453(M+1)⁺

Retention time: 13.10 min (method C)

¹H NMR (200 MHz, DMSO-d6) δ ppm 0.92 (t, 3H) 1.30-1.48 (m, 2H) 1.62-1.78 (m, 2H) 2.33 (t, J=6.64 Hz, 2H) 2.76 (t, 2H) 3.82 (s, 2H) 4.17 (s, 2H) 4.25 (t, 2H) 7.24-7.38 (m, 5H) 7.47 (d, J=8.20 Hz, 2H) 7.86 (d, J=8.20 Hz, 2H)

### Example 85

### N-(4-{5-[butyl(2-fluorobenzoyl)amino]-1,3,4-thiadiazol-2-yl}benzyl)glycine

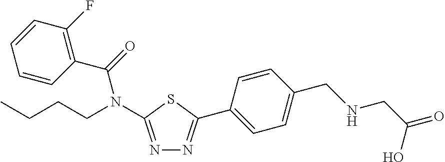

Obtained (2% yield) from the title compound of Preparation 47 and glycine following the procedure described in Example 54.

LRMS: m/z 443(M+1)⁺

Retention time: 14.48 min (method C)

¹H NMR (200 MHz, CHLOROFORM-d) δ ppm 0.68 (t, J=7.03 Hz, 3H) 0.99-1.32 (m, J=13.03, 6.78, 6.78, 6.59 Hz, 2H) 1.48-1.83 (m, 2H) 3.30-3.65 (m, 2H) 3.92-4.33 (m, 4H) 7.10-7.33 (m, 2H) 7.37-7.73 (m, 4H) 7.89 (br. s., 2H)

### Example 86

### Ethyl 1-(4-{5-[butyl(2-fluorobenzoyl)amino]-1,3,4-thiadiazol-2-yl}benzyl)azetidine-3-carboxylate

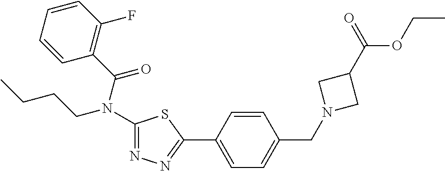

The title compound of Example 54 (3.00 g, 6.4 mmol) was dissolved in HCl 1.25N in ethanol (12 mL) and the solution was stirred at 75° C. for 3 hours and at room temperature overnight. The solvent was evaporated under vacuum and the crude was triturated with diisopropyl ether to give an impure compound which was purified according to purification method A to yield 20 mg of the title compound (0.6%) as a solid.

LRMS: m/z 497(M+1)⁺

Retention time: 12.50 min (method C)

¹H NMR (200 MHz, DMSO-d₆) δ ppm 0.61-0.77 (m, 3H) 1.19 (t, J=7.03 Hz, 5H) 1.53-1.75 (m, J=7.81, 7.42, 7.22, 7.22 Hz, 2H) 3.33 (s, 3H) 3.43 (d, J=4.69 Hz, 2H) 3.62 (s, 2H) 3.98-4.18 (m, 4H) 7.44 (d, J=7.81 Hz, 4H) 7.60-7.84 (m, 2H) 7.94 (d, J=8.20 Hz, 2H)

### Example 87

### 1-[4-(5-{Butyl[(2-fluorophenyl)acetyl]amino}-1,3,4-thiadiazol-2-yl)benzyl]azetidine-3-carboxylic acid

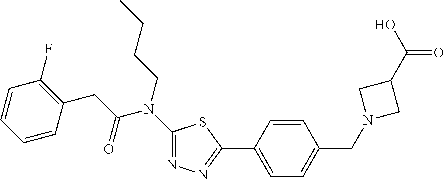

Obtained (28% yield) from the title compound of Preparation 118 and azetidine-3-carboxylic acid following the procedure described in Example 54.

LRMS: m/z 483(M+1)⁺

Retention time: 13.63 min (method C)

¹H NMR (200 MHz, DMSO-d6) δ ppm 0.97 (t, 3H) 1.43 (q, 2H) 1.72-1.88 (m, 2H) 3.52-3.66 (m, 7H) 4.26 (s, 2H) 4.27-4.41 (m, 2H) 7.11-7.30 (m, 3H) 7.31-7.47 (m, 3H) 7.85 (d, J=8.20 Hz, 2H)

### Example 88

### 1-(4-{5-[(Cyclopropylmethyl)(2-methylbenzoyl)amino]-1,3,4-thiadiazol-2-yl}benzyl)azetidine-3-carboxylic acid

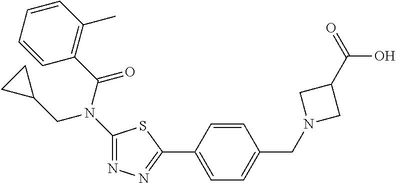

Obtained (10% yield) from the title compound of Preparation 120 and azetidine-3-carboxylic acid following the procedure described in Example 54.

LRMS: m/z 463(M+1)⁺

Retention time: 12.62 min (method C)

¹H NMR (200 MHz, DMSO-d₆) δ ppm 0.53 (d, J=6.64 Hz, 2H) 1.17 (t, J=7.03 Hz, 2H) 1.30-1.51 (m, 1H) 3.12-3.65 (m, 5H) 3.91-4.13 (m, 2H) 4.32 (d, J=6.64 Hz, 2H) 7.41 (d, J=7.81 Hz, 4H) 7.86 (d, J=8.20 Hz, 4H)

### Example 89

### 1-(4-{5-[(Cyclopropylmethyl)(phenylacetyl)amino]-1,3,4-thiadiazol-2-yl}benzyl)azetidine-3-carboxylic acid

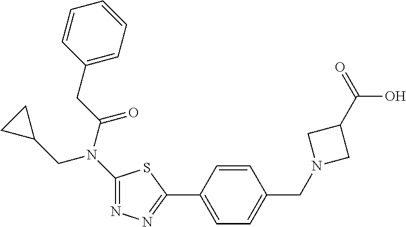

Obtained (24% yield) from the title compound of Preparation 122 and azetidine-3-carboxylic acid following the same procedure described in Example 54.

LRMS: m/z 463(M+1)⁺

Retention time: 12.94 min (method C) ¹H NMR (200 MHz, DMSO-d₆) δ ppm 0.05 (d, J=3.51 Hz, 2H) 0.41 (d, J=7.42 Hz, 2H) 1.04-1.26 (m, 2H) 3.42 (m, 2H) 3.09-3.42 (m, 3H) 3.62 (s, 2H) 3.96 (d, J=6.64 Hz, 2H) 7.27-7.63 (m, 5H) 7.94 (d, J=8.20 Hz, 4H)

### Example 90

### 1-(4-{5-[(Cyclopropylmethyl)(4-methoxybenzoyl)amino]-1,3,4-thiadiazol-2-yl}benzyl)azetidine-3-carboxylic acid

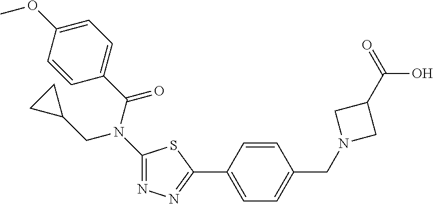

Obtained (29% yield) from the title compound of Preparation 124 and azetidine-3-carboxylic acid following the procedure described for the synthesis of Example 54.

LRMS: m/z 479(M+1)⁺

Retention time: 12.27 min (method C)

¹H NMR (200 MHz, DMSO-d₆) δ ppm 0.05-0.17 (m, 3H) 0.39 (d, J=7.03 Hz, 2 H) 1.18 (t, J=7.22 Hz, 3H) 3.12-3.50 (m, 5H) 3.62 (s, 2H) 3.85 (s, 3H) 4.17 (d, J=6.64 Hz, 2H) 7.09 (d, J=8.98 Hz, 2H) 7.44 (d, J=8.20 Hz, 2H) 7.64 (d, J=8.59 Hz, 2H) 7.92 (d, 2H)

### Example 91

### 1-(4-{5-[Benzoyl(butyl)amino]-1,3,4-thiadiazol-2-yl}benzyl)azetidine-3-carboxylic acid

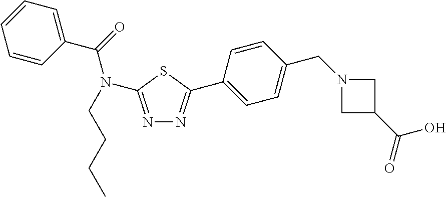

Obtained (20% yield) from the title compound of Preparation 125 and azetidine-3-carboxylic acid following the procedure described in Example 54.

LRMS: m/z 451(M+1)⁺

Retention time: 12.95 min (method C)

¹H NMR (200 MHz, DMSO-d₆) δ ppm 0.71 (t, 3H) 1.02-1.27 (m, 2H) 1.56-1.79 (m, 2H) 3.61 (s, 2H) 4.02-4.21 (m, 2H) 7.44 (d, J=8.20 Hz, 2H) 7.54-7.71 (m, 5H) 7.92 (d, J=8.59 Hz, 2H)

### Example 92

### 1-(4-{5-[Butyl(2-fluorobenzoyl)amino]-1,3,4-thiadiazol-2-yl}-2-methylbenzyl)-azetidine-3-carboxylic acid

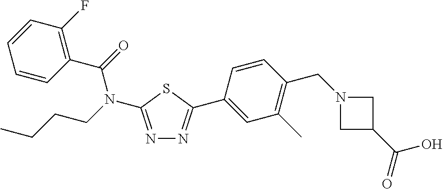

Obtained (21% yield) from the title compound of Preparation 129 and azetidine-3-carboxylic acid following the same procedure described in Example 54.

LRMS: m/z 483(M+1)⁺

Retention time: 13.55 min (method C)

¹H NMR (400 MHz, DMSO-d6) δ ppm 0.70 (t, J=7.24 Hz, 3H) 1.04-1.25 (m, 2H) 1.51-1.77 (m, 2H) 2.35 (s, 3H) 3.17-3.33 (m, 4H) 3.53-3.66 (m, 2H) 3.97-4.14 (m, 2H) 7.27-7.54 (m, 3H) 7.57-7.87 (m, 4H)

### Example 93

### 1-(4-{5-[Butyl(2-chlorobenzoyl)amino]-1,3,4-thiadiazol-2-yl}benzyl)azetidine-3-carboxylic acid

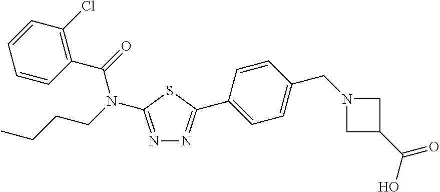

Obtained (48% yield) from the title compound of Preparation 130 and azetidine-3-carboxylic acid following the same procedure described in Example 54.

LRMS: m/z 485(M+1)⁺

Retention time: 13.69 min (method C)

¹H NMR (200 MHz, DMSO-d6) δ ppm 0.49-0.84 (m, 3H) 0.94-1.30 (m, 2H) 1.48-1.87 (m, 2H) 3.08-3.30 (m, 2H) 3.52-3.70 (m, 2H) 3.97-4.32 (m, 2 H) 7.36-7.50 (m, 2H) 7.51-7.83 (m, 3H) 7.85-8.06 (m, 2H)

### Example 94

### 1-(4-{5-[Ethyl(phenylacetyl)amino]-1,3,4-thiadiazol-2-yl}benzyl)azetidine-3-carboxylic acid

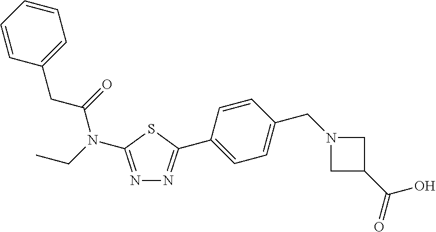

Obtained (20% yield) from the title compound of Preparation 132 and azetidine-3-carboxylic acid following the procedure described in Example 54.

LRMS: m/z 437(M+1)⁺

Retention time: 11.21 min (method C)

¹H NMR (400 MHz, DMSO-d6) δ ppm 1.34 (t, J=7.04 Hz, 3H) 3.17-3.25 (m, 4H) 3.59 (s, 2H) 4.19 (s, 2H) 4.36 (q, J=6.78 Hz, 2H) 7.18-7.50 (m, 7H) 7.85 (d, J=8.22 Hz, 2H)

### Example 95

### 1-(4-{5-[Ethyl(2-methoxybenzoyl)amino]-1,3,4-thiadiazol-2-yl}benzyl)azetidine-3-carboxylic acid

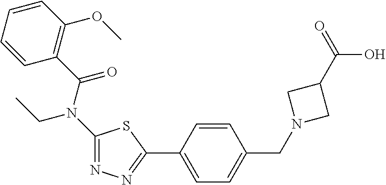

Obtained (5% yield) from the title compound of Preparation 135 and azetidine-3-carboxylic acid following the procedure described for the synthesis of Example 54.

LRMS: m/z 453(M+1)⁺

Retention time: 11.14 min (method C)

¹H NMR (400 MHz, DMSO-d₆) δ ppm 1.18 (t, J=6.85 Hz, 3H) 3.13-3.52 (m, 5 H) 3.62 (br. s., 2H) 3.84 (s, 3H) 4.14 (br. s., 2H) 7.12 (t, J=7.43 Hz, 2H) 7.23 (d, J=8.22 Hz, 1H) 7.45 (d, J=13.69 Hz, 2H) 7.56 (d, J=15.26 Hz, 2H) 7.92 (d, J=7.83 Hz, 1H)

### Example 96

### 1-(4-{5-[(2-Chlorobenzoyl)(ethyl)amino]-1,3,4-thiadiazol-2-yl}benzyl)azetidine-3-carboxylic acid

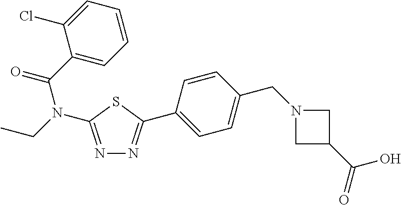

Obtained (7% yield) from the title compound of Preparation 137 and azetidine-3-carboxylic acid following the procedure described for the synthesis of Example 54.

LRMS: m/z 457(M+1)⁺

Retention time: 11.90 min (method C)

¹H NMR (200 MHz, DMSO-d₆) δ ppm 1.23 (t, J=6.25 Hz, 3H) 3.22 (br. s., 3H) 3.40 (d, J=4.69 Hz, 2H) 3.62 (br. s., 1H) 4.13 (br. s., 2H) 7.44 (d, J=7.42 Hz, 2H) 7.51-7.82 (m, 4H) 7.94 (d, J=7.42 Hz, 2H)

### Example 97

### 1-(4-{5-[Butyl(2-fluorobenzoyl)amino]-1,3,4-thiadiazol-2-yl}-3-methylbenzyl)-pyrrolidine-3-carboxylic acid

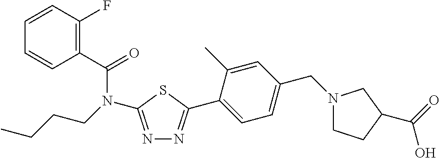

Obtained (19% yield) from the title compound of Preparation 111 and pyrrolidine-3-carboxylic acid following the procedure described for the synthesis of Example 54.

LRMS: m/z 497(M+1)⁺

Retention time: 13.10 min (method C)

¹H NMR (200 MHz, DMSO-d6) δ ppm 0.70 (t, 3H) 1.07-1.24 (m, 2H) 1.67 (t, J=8.39 Hz, 2H) 1.86-2.05 (m, 2H) 2.54 (s, 3H) 2.57-2.81 (m, 2H) 2.84-3.03 (m, 2H) 3.61 (s, 3H) 4.08 (t, 2H) 7.24-7.54 (m, 4H) 7.59-7.79 (m, 3H)

### Example 98

### 1-(4-{5-[Benzoyl(ethyl)amino]-1,3,4-thiadiazol-2-yl}benzyl)azetidine-3-carboxylic acid

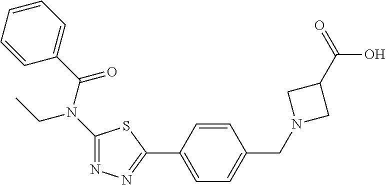

Obtained (29% yield) from the title compound of Preparation 138 and azetidine-3-carboxylic acid following the procedure described in Example 54.

LRMS: m/z 423(M+1)⁺

Retention time: 11.04 min (method C)

¹H NMR (400 MHz, DMSO-d₆) δ ppm 1.26 (t, J=6.85 Hz, 3H) 3.27 (br. s., 3H) 3.46 (br. s., 2H) 3.66 (br. s., 2H) 4.12 (q, J=7.04 Hz, 2H) 7.45 (d, J=7.83 Hz, 2 H) 7.51-7.61 (m, 3H) 7.94 (d, J=8.22 Hz, 2H).

### Example 99

### 1-(4-{5-[Butyl(2-fluorobenzoyl)amino]-1,3,4-thiadiazol-2-yl}benzyl)pyrrolidine-3-carboxylic acid

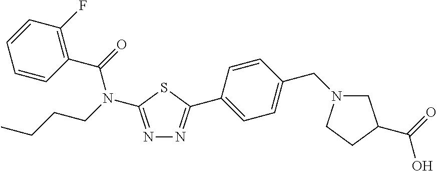

Obtained (23% yield) from the title compound of Preparation 47 and pyrrolidine-3-carboxylic acid following the procedure described in Example 54.

LRMS: m/z 483(M+1)⁺

Retention time: 11.65 min (method C)

¹H NMR (200 MHz, DMSO-d6) δ ppm 0.73 (t, J=7.22 Hz, 3H) 1.09-1.25 (m, 2H) 1.58-1.76 (m, 2H) 1.90-2.09 (m, 3H) 2.63-2.79 (m, 2H) 2.89-3.06 (m, 2H) 3.68 (br. s., 2H) 4.08 (t, J=8.59 Hz, 2H) 7.39-7.58 (m, 4H) 7.64-7.81 (m, 2H) 7.91-8.04 (m, 2H)

### Example 100

### 1-(4-{5-[Butyl(phenylacetyl)amino]-1,3,4-thiadiazol-2-yl}-3-methylbenzyl)azetidine-3-carboxylic acid

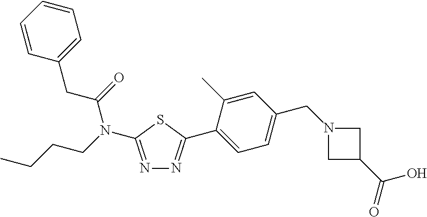

Obtained (9% yield) from the title compound of Preparation 140 and azetidine-3-carboxylic acid following the same procedure described in Example 54.

LRMS: m/z 479(M+1)⁺

Retention time: 14.08 min (method C)

¹H NMR (200 MHz, DMSO-d₆) δ ppm 0.94 (t, J=7.22 Hz, 3H) 1.26-1.52 (m, 2H) 1.57-1.82 (m, 2H) 3.20 (br. s., 4H) 3.56 (br. s., 2H) 3.99-4.40 (m, 4H) 7.08-7.42 (m, 7H) 7.60 (d, J=7.42 Hz, 1H)

### Example 101

### 1-[4-(5-{Butyl[(2-chlorophenyl)acetyl]amino}-1,3,4-thiadiazol-2-yl)-3-methyl-benzyl]azetidine-3-carboxylic acid

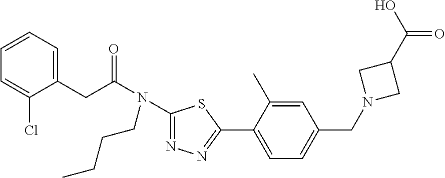

Obtained (66% yield) from the title compound of Preparation 144 and azetidine-3-carboxylic acid following the procedure described for the synthesis of Example 54.

LRMS: m/z 514(M+1)⁺

Retention time: 15.15 min (method C)

¹H NMR (400 MHz, DMSO-d6) δ ppm 0.99 (t, J=7.26 Hz, 3H) 1.39-1.55 (m, J=14.88, 7.39, 7.39, 7.26 Hz, 2H) 1.79-1.94 (m, 2H) 2.48 (s, 3H) 3.15-3.26 (m, 4H) 3.56 (s, 2H) 4.28-4.42 (m, 4H) 7.22 (d, J=7.88 Hz, 1H) 7.28 (s, 1H) 7.31-7.40 (m, 2H) 7.36 (qd, 2H) 7.41-7.52 (m, 2H) 7.59 (d, J=7.88 Hz, 1H)

### Example 102

### 1-(4-{5-[Ethyl(3-fluorobenzoyl)amino]-1,3,4-thiadiazol-2-yl}benzyl)azetidine-3-carboxylic acid

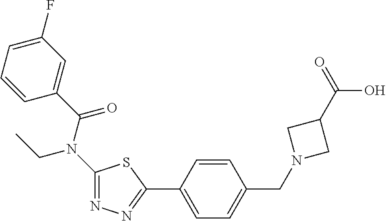

Obtained (10% yield) from the title compound of Preparation 145 and azetidine-3-carboxylic acid following the procedure described in Example 54.

LRMS: m/z 441(M+1)⁺

Retention time: 11.55 min (method C)

¹H NMR (200 MHz, DMSO-d₆) δ ppm 1.25 (d, J=5.86 Hz, 3H) 3.19 (d, J=9.76 Hz, 5H) 3.61 (br. s., 2H) 4.08 (br. s., 2H) 7.44 (d, J=7.42 Hz, 3H) 7.60 (d, J=7.03 Hz, 2H) 7.93 (d, J=7.42 Hz, 2H).

### Example 103

### 1-[4-(5-{Butyl[(2-chlorophenyl)acetyl]amino}-1,3,4-thiadiazol-2-yl)-3-methyl-benzyl]pyrrolidine-3-carboxylic acid

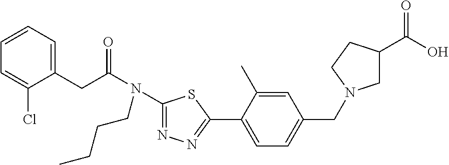

Obtained (10% yield) from the title compound of Preparation 144 and pyrrolidine-3-carboxylic acid following the procedure described in Example 54.

LRMS: m/z 528(M+1)⁺

Retention time: 14.52 min (method C)

¹H NMR (400 MHz, DMSO-d6) δ ppm 0.99 (t, J=7.43 Hz, 3H) 1.37-1.56 (m, 3 H) 1.78-1.92 (m, 2H) 1.92-2.01 (m, 2H) 2.49 (s, 3H) 2.60-2.69 (m, 1H) 2.70-2.80 (m, 1H) 2.84-3.02 (m, 2H) 3.52-3.66 (m, 3H) 4.23-4.45 (m, 4 H) 7.26 (d, J=8.61 Hz, 1H) 7.30-7.40 (m, 3H) 7.40-7.53 (m, 2H) 7.61 (d, J=7.83 Hz, 1H)

### Example 104

### 1-(4-{5-[butyl(2-chlorobenzoyl)amino]-1,3,4-thiadiazol-2-yl}-3-methylbenzyl)-azetidine-3-carboxylic acid

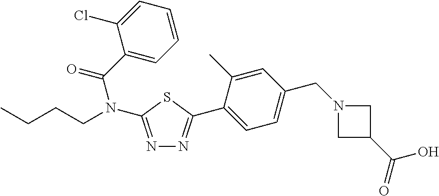

Obtained (25% yield) from the title compound of Preparation 147 and azetidine-3-carboxylic acid following the procedure described in Example 54.

LRMS: m/z 499(M+1)⁺

Retention time: 14.28 min (method C)

¹H NMR (200 MHz, DMSO-d₆) δ ppm 0.69 (t, J=7.22 Hz, 3H) 0.95-1.38 (m, 2H) 1.39-1.96 (m, 2H) 2.52 (s, 3H) 3.03-3.31 (m, 4H) 3.32-3.48 (m, 2H) 3.49-3.65 (m, 2H) 6.97-7.40 (m, 2H) 7.40-7.93 (m, 5H)

### Example 105

### N-{5-[4-(2-Aminoethoxy)-3,5-dimethylphenyl]-1,3,4-thiadiazol-2-yl}-N-butyl-2-fluorobenzamide

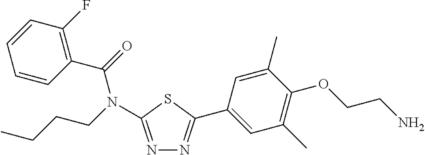

To a 0° C. cooled solution of the title compound of Preparation 148 (0.54 g, 1.28 mmol) in THF (6 mL), sodium hydride (108 mg, 2.70 mmol) was added and the reaction mixture was stirred at 0° C. for 30 min. 2-Fluorobenzoil chloride (428 mg, 2.70 mmol) in THF (5 mL) was added and the reaction mixture was stirred at 0° C. for 30 min and at room temperature for 2 hours. Acetic acid (5 mL) in water (25 mL) was added, it was extracted with ethyl acetate (×2), the organic layers were washed with water, brine, dried, filtered and the solvent concentrated in vacuo. The crude was dissolved in dioxane (25 mL), 4M hydrochloric acid in dioxane (2 mL, 8 mmol) was added and the reaction mixture was stirred at room temperature for 48 hours. The solid was filtered off, washed with diethyl ether and dried to yield 335 mg (59% yields) of the title compound as a white crystalline solid.

LRMS: m/z 443(M+1)⁺

Retention time: 12.83 min (method C)

¹H NMR (200 MHz, DMSO-d6) d ppm 0.70 (t, J=7.42 Hz, 3H) 1.03-1.25 (m, 2H) 1.53-1.76 (m, 2H) 2.26-2.41 (s, 6H) 3.17-3.35 (m, 2H) 3.90-4.15 (m, J=5.08, 5.08 Hz, 4H) 7.35-7.55 (m, 2H) 7.60-7.80 (m, 4H) 8.11-8.35 (m, 3H)

### Example 106

### 1-(4-{5-[butyl(2-fluorobenzoyl)amino]-1,3,4-thiadiazol-2-yl}-2,6-dimethylbenzyl)-azetidine-3-carboxylic acid

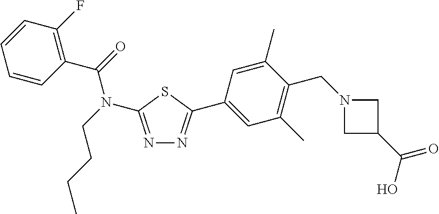

Obtained (35% yield) from the title compound of Preparation 152 and azetidine-3-carboxylic acid following the procedure described in Example 54.

LRMS: m/z 497(M+1)⁺

Retention time: 14.34 min (method C)

¹H NMR (400 MHz, DMSO-d6) δ ppm 0.71 (t, J=7.43 Hz, 3H) 1.08-1.22 (m, 2 H) 1.57-1.74 (m, 2H) 2.45 (s, 6H) 3.13-3.21 (m, 1H) 3.26 (t, J=6.46 Hz, 3H) 3.41 (t, J=6.85 Hz, 3H) 3.97-4.11 (m, 2H) 7.36-7.54 (m, 2H) 7.57-7.81 (m, 4H)

### Example 107

### N-Butyl-N-{5-[4-(2,3-dihydroxypropoxy)-3,5-dimethylphenyl]-1,3,4-thiadiazol-2-yl}-2-methylbenzamide

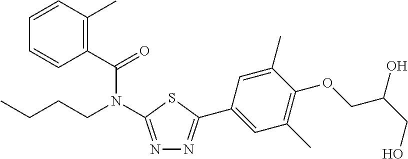

Obtained (89% yield) from the title compound of Preparation 155 following the procedure described in Preparation 78.

LRMS: m/z 470(M+1)⁺

Retention time: 17.68 min (method C)

¹H NMR (300 MHz, DMSO-d6) δ ppm 0.69 (t, J=7.42 Hz, 3H) 1.03-1.20 (m, 2H) 1.54-1.74 (m, J=6.87 Hz, 2H) 2.26 (s, 3H) 2.33 (s, 6H) 3.43-3.55 (m, 2H) 3.66-3.78 (m, 1H) 3.78-3.89 (m, 4H) 4.69 (s, 1H) 5.02 (s, 1H) 7.31-7.43 (m, 2H) 7.44-7.57 (m, 2H) 7.67 (s, 2H)

### Example 108

### 1-(4-{5-[(3-Chloro-2-fluorobenzoyl)(ethyl)amino]-1,3,4-thiadiazol-2-yl}benzyl)-azetidine-3-carboxylic acid

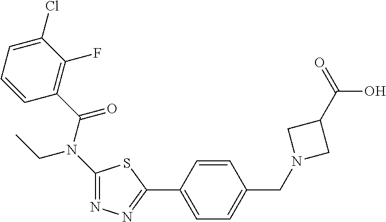

Obtained (19% yield) from the title compound of Preparation 157 and azetidine-3-carboxylic acid following the experimental procedure described in Example 54.

LRMS: m/z 475(M+1)⁺

Retention time: 12.38 min (method C)

¹H NMR (400 MHz, DMSO-d6) δ 1.09 (t, J=7.0 Hz, 3H), 1.25 (t, J=6.9 Hz, 2H), 3.62 (m, 2H), 4.11 (dd, J=13.6, 6.6 Hz, 1H), 4.17 (d, J=25.3 Hz, 2H), 4.48 (d, J=10.3 Hz, 1H), 7.47 (t, J=7.9 Hz, 0H), 7.68 (t, J=8.9 Hz, 1H), 7.71 (s, 1H), 7.87 (t, J=7.1 Hz, 1H), 8.10 (d, J=8.1 Hz, 1H).

### Example 109

### 1-(4-{5-[Butyl(3-chloro-2-fluorobenzoyl)amino]-1,3,4-thiadiazol-2-yl}-3-methyl-benzyl)piperidine-4-carboxylic acid

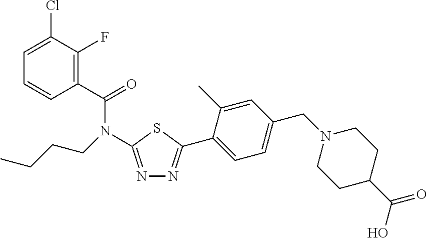

Obtained (27% yield) from the title compound of Preparation 160 and piperidine-4-carboxylic acid following the procedure described in Example 54.

LRMS: m/z 546(M+1)⁺

Retention time: 13.48 min (method C)

¹H NMR (400 MHz, DMSO-d6) δ ppm 0.73 (t, J=7.42 Hz, 3H) 1.12-1.24 (m, J=14.61, 7.57, 7.57, 7.38 Hz, 2H) 1.69 (quin, J=7.52 Hz, 2H) 1.76-1.90 (m, 2 H) 2.00-2.11 (m, 2H) 2.57 (s, 3H) 3.36 (br. s., 4H) 4.06 (t, 2H) 4.29-4.35 (m, 1H) 4.35-4.40 (m, 0H) 7.47 (t, J=8.01 Hz, 1H) 7.53-7.68 (m, 2H) 7.75 (t, J=7.82 Hz, 1H) 7.81-7.91 (m, 2H)

### Example 110

### 1-(4-{5-[Butyl(3-chloro-2-fluorobenzoyl)amino]-1,3,4-thiadiazol-2-yl}-3-methyl-benzyl)azetidine-3-carboxylic acid

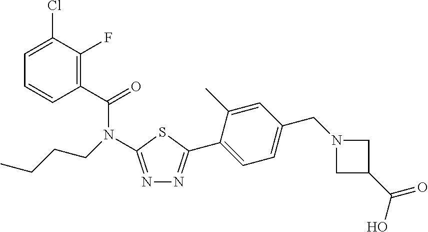

Obtained (20% yield) from the title compound of Preparation 160 and azetidine-3-carboxylic acid following the procedure described in Example 54.

LRMS: m/z 518(M+1)⁺

Retention time: 14.80 min (method C)

¹H NMR (400 MHz, DMSO-d6) δ ppm 0.72 (t, J=7.42 Hz, 3H) 1.12-1.24 (m, 2H) 1.62-1.74 (m, J=7.82, 7.52, 7.38, 7.38 Hz, 2H) 2.56 (s, 3H) 3.36-3.43 (m, 1H) 3.54-3.69 (m, 2H) 4.06 (t, 2H) 4.15-4.21 (m, 2H) 4.40 (br. s., 2H) 7.42-7.54 (m, 2H) 7.57 (s, 1H) 7.71-7.78 (m, 1H) 7.80 (d, J=8.21 Hz, 1H) 7.87 (t, J=7.82 Hz, 1H)

### Example 111

### N-Butyl-3-chloro-N-{5-[4-(2,3-dihydroxypropoxy)-3,5-dimethylphenyl]-1,3,4-thiadiazol-2-yl}-2-fluorobenzamide

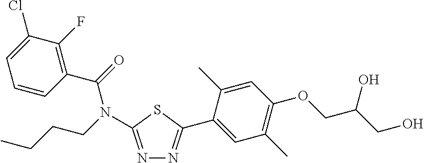

Obtained (95% yield) from the title compound of Preparation 161 following the experimental procedure described in Preparation 78.

LRMS: m/z 509(M+1)⁺

Retention time: 18.34 min (method C)

¹H NMR (400 MHz, DMSO-d6) δ ppm 0.72 (t, J=7.42 Hz, 3H) 1.10-1.22 (m, 2H) 1.60-1.72 (m, 2H) 2.33 (s, 6H) 3.49 (t, J=5.67 Hz, 2H) 3.69-3.78 (m, 1H) 3.77-3.90 (m, 2H) 3.99-4.09 (m, 2H) 4.64 (t, J=5.67 Hz, 1H) 4.96 (d, J=5.08 Hz, 1H) 7.45 (t, J=7.82 Hz, 1H) 7.68 (s, 2H) 7.74 (t, J=6.25 Hz, 1H) 7.82-7.90 (m, 1H)

### Example 112

### 1-(4-{5-[(3-chloro-2-fluorobenzoyl)(ethyl)amino]-1,3,4-thiadiazol-2-yl}benzyl)-piperidine-4-carboxylic acid

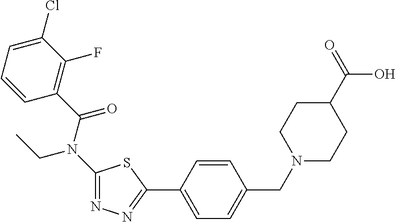

Obtained (89% yield) from the title compound of Preparation 157 and piperidine-4-carboxylic acid following the procedure described in Example 54.

LRMS: m/z 503(M+1)⁺

Retention time: 11.55 min (method C)

### Example 113

### N-{5-[4-(3-Amino-2-hydroxypropoxy)-3,5-dimethylphenyl]-1,3,4-thiadiazol-2-yl}-N-butyl-2-fluorobenzamide

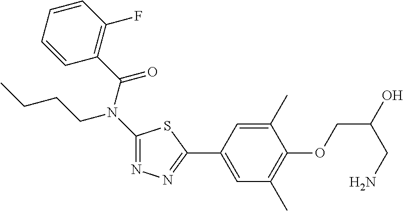

To a solution of the title compound of Preparation 159 (123 mg, 0.20 mmol) in THF (5 mL) and methanol (5 mL), Pd/C 10% was added (180 mg) and the reaction mixture was hydrogenated at 28 psi for 20 hours. The catalyst was filtered and the solvent was evaporated under vacuum to yield a crude product that was purified following purification method A to give 43 mg (43%) of the title compound.

LRMS: m/z 473(M+1)⁺

Retention time: 12.48 min (method C)

¹H NMR (400 MHz, DMSO-d6) δ 0.70 (t, J=7.3 Hz, 3H), 1.34 (m, 2H), 1.65 (m, 2H), 2.33 (m, 6H), 3.80 (m, 1H), 4.07 (m, 3H), 7.08 (m, 1H), 7.41 (m, 2H), 7.73 (m, 1H), 7.99 (s, 1H), 8.06 (dt, J=24.3, 17.7 Hz, 1H), 8.25 (t, J=11.7 Hz, 1H).

### Example 114

### 1-(4-{5-[(3-Chloro-2-fluorobenzoyl)(cyclopropylmethyl)amino]-1,3,4-thiadiazol-2-yl}benzyl)piperidine-4-carboxylic acid

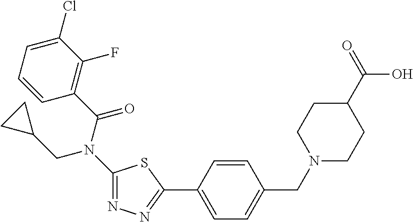

Obtained (6% yield) from the title compound of Preparation 163 and piperidine-6-carboxylic acid following the procedure described in Example 54.

LRMS: m/z 530(M+1)⁺

Retention time: 12.51 min (method C)

¹H NMR (400 MHz, DMSO-d6) δ 0.13 (d, J=4.2 Hz, 2H), 0.44 (d, J=7.5 Hz, 2H), 1.11 (dd, J=24.6, 17.6 Hz, 2H), 1.81 (d, J=11.9 Hz, 1H), 2.06 (d, J=13.0 Hz, 2H), 2.97 (d, J=10.5 Hz, 1H), 3.39 (dd, J=17.8, 10.7 Hz, 2H), 4.05 (d, J=6.4 Hz, 1H), 4.39 (d, J=24.1 Hz, 1H), 7.41 (m, 1H), 7.73 (m, 1H), 7.99 (m, 1H), 8.06 (m, 1H).

### Example 115

### 1-(4-{5-[Butyl(3-chloro-2-fluorobenzoyl)amino]-1,3,4-thiadiazol-2-yl}-2,6-dimethyl-benzyl)azetidine-3-carboxylic acid

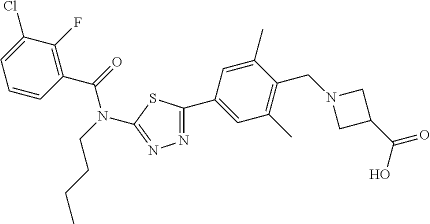

Obtained (16% yield) from the title compound of Preparation 166 and azetidine-3-carboxylic acid following the procedure described for the synthesis of Example 54.

LRMS: m/z 532(M+1)⁺

Retention time: 15.13 min (method C)

¹H NMR (400 MHz, DMSO-d₆) δ ppm 0.72 (t, J=7.23 Hz, 3H) 1.04-1.25 (m, 2H) 1.67 (quin, J=7.42 Hz, 2H) 2.50 (s, 6H) 3.35 (m, 3H) 4.05 (t, 2H) 4.12-4.74 (m, 2H) 7.47 (t, J=7.82 Hz, 1H) 7.65-7.83 (m, 3H) 7.82-7.97 (m, 1H)

### Example 116

### 1-(4-{5-[Butyl(3-chloro-2-fluorobenzoyl)amino]-1,3,4-thiadiazol-2-yl}-2,6-dimethyl-benzyl)piperidine-4-carboxylic acid

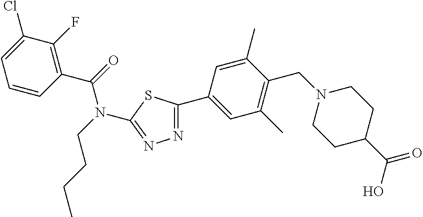

Obtained (68% yield) from the title compound of Preparation 166 and piperidine-4-carboxylic acid following the experimental procedure described in Example 54.

LRMS: m/z 560(M+1)⁺

Retention time: 13.78 min (method C)

¹H NMR (400 MHz, DMSO-d₆) δ ppm 0.72 (t, J=7.42 Hz, 3H) 1.09-1.25 (m, 2H) 1.57-1.76 (m, 2H) 1.79-2.21 (m, 5H) 2.55 (s, 6H) 3.11-3.56 (m, 4H) 4.05 (t, 2H) 4.40 (d, J=4.69 Hz, 2H) 7.47 (t, J=7.82 Hz, 1H) 7.76 (t, 1H) 7.82 (s, 2H) 7.88 (td, J=7.82, 1.56 Hz, 1H)

### Example 117

### 1-(4-{5-[Butyl(2-chlorobenzoyl)amino]-1,3,4-thiadiazol-2-yl}-2,6-dimethylbenzyl)-azetidine-3-carboxylic acid

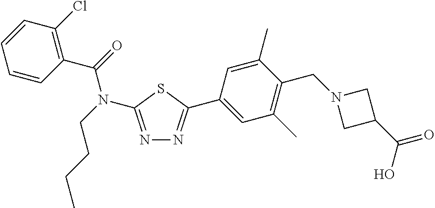

Obtained (20% yield) from the title compound of Preparation 169 and azetidine-3-carboxylic acid following the procedure described in Example 54.

LRMS: m/z 514 (M+1)⁺

Retention time: 14.43 min (method C)

¹H NMR (400 MHz, DMSO-d₆) δ ppm 0.41-0.88 (m, 3H) 0.99-1.34 (m, 2H) 1.45-1.90 (m, 2H) 2.45 (s, 6H) 3.04-3.51 (m, 6H) 3.61 (t, 2H) 7.14-8.11 (m, 6H)

### Example 118

### 1-(4-{5-[Butyl(2-chlorobenzoyl)amino]-1,3,4-thiadiazol-2-yl}-3-methylbenzyl)-piperidine-4-carboxylic acid

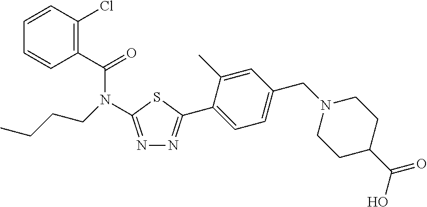

Obtained (18% yield) from the title compound of Preparation 147 and piperidine-4-carboxylic acid following the procedure described in Example 54.

LRMS: m/z 528(M+1)⁺

Retention time: 12.35 min (method C)

¹H NMR (400 MHz, DMSO-d6) δ ppm 0.70 (t, J=7.23 Hz, 3H) 1.08-1.21 (m, 2H) 1.59-1.86 (m, 4H) 2.05 (br. s., 4H) 2.55-2.61 (m, 3H) 2.91-3.05 (m, 2H) 3.29 (br. s., 1H) 3.43 (d, J=12.90 Hz, 1H) 3.84 (br. s., 1H) 4.15 (br. s., 1H) 4.28-4.42 (m, 2H) 7.51-7.66 (m, 4H) 7.68-7.72 (m, 1H) 7.77 (d, J=7.42 Hz, 1H) 7.82-7.88 (m, 1H)

### Example 119

### 1-(4-{5-[Butyl(2-chlorobenzoyl)amino]-1,3,4-thiadiazol-2-yl}-2,6-dimethylbenzyl)-piperidine-4-carboxylic acid

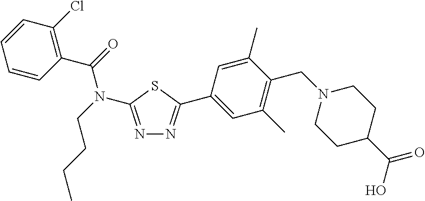

Obtained (7% yield) from the title compound of Preparation 169 and piperidine-4-carboxylic acid following the procedure described in Example 54.

LRMS: m/z 542(M+1)⁺

Retention time: 13.85 min (method C)

¹H NMR (400 MHz, DMSO-d₆) δ ppm 0.69 (t, J=7.23 Hz, 3H) 1.05-1.25 (m, 2H) 1.54-1.81 (m, 2H) 1.81-2.18 (m, 4H) 2.55 (s, 6H) 3.16-3.33 (m, 2H) 3.42-3.53 (m, 2H) 3.74-3.94 (m, 1H) 4.04-4.24 (m, 1H) 4.30-4.53 (m, 2H) 7.43-7.93 (m, 6H)

### Pharmacological Activity

### 35S-GTP-g Binding Assay:

The effect of the compounds was measured using a 35S-GTPyS binding assay. Briefly, membranes were incubated in a buffer containing 20 mM HEPES pH 7.4, 100 mM NaCl, 10 mM MgCl2, 10 μM GDP, 50 μg/ml saponin and 0.2% fatty acid-free BSA at various concentrations (0.1 nM-10 μM) and 0.1 nM 35S-GTPyS. 10 μM S1P was used as 100% maximum efficacy. The assay was incubated for 90 min at room temperature with gentle mixing, and terminated by filtrating the reaction mixture through GF/C filter plates using the Manifold Filtration System. The filters were immediately washed with sodium phosphate pH 7.4 buffers. After drying the filter plate's scintillant liquid were added to each well and 35S-GTPyS binding was measured on a Trilux Scintillation Counter.

The results are shown in Table 1.

TABLE 1

| EXAMPLES | EC₅₀ (nM) |
|---|---|
| 8 | 6.2 |
| 9 | 4.4 |
| 19 | 8.3 |
| 31 | 3.4 |
| 32 | 64.2 |
| 39 | 3.8 |
| 46 | 36.0 |
| 48 | 115.5 |
| 49 | 88.7 |
| 53 | 46.3 |
| 59 | 2.3 |
| 69 | 55 |
| 87 | 1.4 |
| 97 | 2 |
| 105 | 0.45 |
| 108 | 7.0 |
| 111 | 0.15 |
| 113 | 1.52 |
| 118 | 4.42 |

The 2-aminothiadiazole derivatives of the invention may also be combined with other active compounds in the treatment of diseases known to be susceptible to improvement by treatment with a sphingosine-1-phosphate receptor agonist (S1P1).

The combinations of the invention can optionally comprise one or more additional active substances which are known to be useful in the treatment of autoimmune diseases, chronic immune and inflammatory diseases, transplant rejection, malignant neoplastic diseases, angiogenic-related disorders, pain, neurological diseases, viral and infectious diseases, such as (a) beta interferons such as Betaseron, Avonex or Rebif, (b), immunomodulators such as glatiramer acetate, (c) inhibitors of DNA synthesis and repair, such as Mitoxantrone, (d) anti-alpha 4 integrin antibodies, such as Natalizumab (Tysabri), (e) alpha 4 integrin antagonists such as R-1295, TBC-4746, CDP-323, ELND-002, Firategrast and TMC-2003, (f), dyhydrofolate reductase inhibitors, such as Methotrexate or CH-1504, (g) glucocorticoids such as prednisone or methylprednisolone, (h), DHODH inhibitors such as Teriflunomide, (i) fumaric acid esters, such as BG-12, (j) immunomodulators such as Laquinimod, (k) anti-CD20 monoclonal antibodies such as Rituximab, Ocrelizumab Ofatumumab or TRU-015, (I) anti-CD52 such as alemtuzumab, (m) anti-CD25 such as daclizumab, (n) anti-CD88, such as eculizumab or pexilizumab, (o) calcineurin inhibitors such as cyclosporine A or tacrolimus, (p) IMPDH inhibitors, such as mycophenolate mophetyl, (q) cannabinoid receptor agonists such as Sativex, (r) chemokine CCR1 antagonists such as MLN-3897 or PS-031291, (s) chemokine CCR2 antagonists such as INCB-8696, (t) interferon alpha such as Sumiferon MP, (u) NF-kappaB activation inhibitors such as FAE and MLN-0415, (v) JAK inhibitors such as CP-690550 or INCB018424, (W) Syk inhibitors, such as R-112, (x) PKC inhibitors, such as NVP-AEB071, (y) phosphosdiesterase IV inhibitors such as GRC-4039, (z) P38 Inhibitors such as ARRY-797, and (aa) MEK inhibitors, such as ARRY-142886 or ARRY-438162

The combinations of the invention may be used in the treatment of disorders which are susceptible to amelioration by sphingosine-1-phosphate receptors agonists (S1P1). Thus, the present application encompasses methods of treatment of these disorders, as well as the use of the combinations of the invention in the manufacture of a medicament for the treatment of these disorders.

Preferred examples of such disorders are multiple sclerosis, transplant rejection, systemic lupus erythematosus, asthma, psoriasis, rheumatoid arthritis, psoriatic arthritis and Crohn's disease, more preferably multiple sclerosis, transplant rejection, asthma and rheumatoid arthritis, and most preferably multiple sclerosis.

The active compounds in the combinations of the invention may be administered by any suitable route, depending on the nature of the disorder to be treated, e.g. orally (as syrups, tablets, capsules, lozenges, controlled-release preparations, fast-dissolving preparations, etc); topically (as creams, ointments, lotions, nasal sprays or aerosols, etc); by injection (subcutaneous, intradermic, intramuscular, intravenous, etc.) or by inhalation (as a dry powder, a solution, a dispersion, etc).

The active compounds in the combination, i.e. the sphingosine-1-phosphate agonist of the invention, and the other optional active compounds may be administered together in the same pharmaceutical composition or in different compositions intended for separate, simultaneous, concomitant or sequential administration by the same or a different route.

One execution of the present invention consists of a kit of parts comprising a sphingosine-1-phosphate agonist of the invention together with instructions for simultaneous, concurrent, separate or sequential use in combination with another active compound useful in the treatment of multiple sclerosis, transplant rejection, systemic lupus erythematosus, asthma, psoriasis, rheumatoid arthritis, psoriatic arthritis and Crohn's disease,

Another execution of the present invention consists of a package comprising a sphingosine-1-phosphate agonist of formula (I) and another active compound useful in the treatment of multiple sclerosis, transplant rejection, systemic lupus erythematosus, asthma, psoriasis, rheumatoid arthritis, psoriatic arthritis and Crohn's disease,

The pharmaceutical formulations may conveniently be presented in unit dosage form and may be prepared by any of the methods well known in the art of pharmacy.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A syrup formulation will generally consist of a suspension or solution of the compound or salt in a liquid carrier for example, ethanol, peanut oil, olive oil, glycerine or water with flavouring or colouring agent.

Where the composition is in the form of a tablet, any pharmaceutical carrier routinely used for preparing solid formulations may be used. Examples of such carriers include magnesium stearate, talc, gelatine, acacia, stearic acid, starch, lactose and sucrose.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein.

Where the composition is in the form of a capsule, any routine encapsulation is suitable, for example using the aforementioned carriers in a hard gelatine capsule. Where the composition is in the form of a soft gelatine capsule any pharmaceutical carrier routinely used for preparing dispersions or suspensions may be considered, for example aqueous gums, celluloses, silicates or oils, and are incorporated in a soft gelatine capsule.

Dry powder compositions for topical delivery to the lung by inhalation may, for example, be presented in capsules and cartridges of for example gelatine or blisters of for example laminated aluminium foil, for use in an inhaler or insufflator. Formulations generally contain a powder mix for inhalation of the compound of the invention and a suitable powder base (carrier substance) such as lactose or starch. Use of lactose is preferred. Each capsule or cartridge may generally contain between 2 μg and 150 μg of each therapeutically active ingredient. Alternatively, the active ingredient (s) may be presented without excipients.

Packaging of the formulation for inhalation may be carried out by using suitable inhaler devices such as the Novolizer SD2FL which is described in the following patent applications: WO 97/000703, WO 03/000325 and WO 03/061742.

Typical compositions for nasal delivery include those mentioned above for inhalation and further include non-pressurized compositions in the form of a solution or suspension in an inert vehicle such as water optionally in combination with conventional excipients such as buffers, anti-microbials, tonicity modifying agents and viscosity modifying agents which may be administered by nasal pump.

Typical dermal and transdermal formulations comprise a conventional aqueous or non-aqueous vehicle, for example a cream, ointment, lotion or paste or are in the form of a medicated plaster, patch or membrane.

Preferably the composition is in unit dosage form, for example a tablet, capsule or metered aerosol dose, so that the patient may administer a single dose.

The amount of each active which is required to achieve a therapeutic effect will, of course, vary with the particular active, the route of administration, the subject under treatment, and the particular disorder or disease being treated.

Effective doses are normally in the range of 2-2000 mg of active ingredient per day. Daily dosage may be administered in one or more treatments, preferably from 1 to 4 treatments, per day. Preferably, the active ingredients are administered once or twice a day.

When combinations of actives are used, it is contemplated that all active agents would be administered at the same time, or very close in time. Alternatively, one or two actives could be taken in the morning and the other (s) later in the day. Or in another scenario, one or two actives could be taken twice daily and the other (s) once daily, either at the same time as one of the twice-a-day dosing occurred, or separately. Preferably at least two, and more preferably all, of the actives would be taken together at the same time. Preferably, at least two, and more preferably all actives would be administered as an admixture.

The following preparations forms are cited as formulation examples:

### Composition Example 1

50,000 capsules, each containing 100 mg of N-butyl-N-[5-(4-methoxy-3,5-dimethylphenyl)-1,3,4-thiadiazol-2-yl]-1-naphthamide (active ingredient), were prepared according to the following formulation:

| | |
|---|---|
| Active ingredient | 5 Kg |
| Lactose monohydrate | 10 Kg |
| Colloidal silicon dioxide | 0.1 Kg |
| Corn starch | 1 Kg |
| Magnesium stearate | 0.2 Kg |

### Procedure

The above ingredients were sieved through a 60 mesh sieve, and were loaded into a suitable mixer and filled into 50,000 gelatine capsules.

### Composition Example 2

50,000 tablets, each containing 50 mg of N-butyl-N-[5-(4-methoxy-3,5-dimethylphenyl)-1,3,4-thiadiazol-2-yl]-1-naphthamide (active ingredient), were prepared from the following formulation:

| | |
|---|---|
| Active ingredient | 2.5 Kg |
| Microcrystalline cellulose | 1.95 Kg |
| Spray dried lactose | 9.95 Kg |
| Carboxymethyl starch | 0.4 Kg |
| Sodium stearyl fumarate | 0.1 Kg |
| Colloidal silicon dioxide | 0.1 Kg |

### Procedure

All the powders were passed through a screen with an aperture of 0.6 mm, then mixed in a suitable mixer for 20 minutes and compressed into 300 mg tablets using 9 mm disc and flat bevelled punches. The disintegration time of the tablets was about 3 minutes.

1. A compound of formula (I): 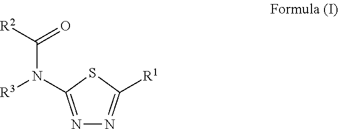
wherein
R¹ is chosen from:
8 to 10 membered bicyclic N-containing heteroaryl groups optionally substituted by one or more substitutents chosen from halogen atoms, hydroxycarbonyl groups, C₁₋₄ alkyl groups, C₁₋₄ haloalkyl groups, C₁₋₄ alkoxy groups and C₃₋₄ cycloalkyl groups;
pyridyl groups substituted with one or more substituents chosen from halogen atoms, hydroxy groups, hydroxycarbonyl groups, C₁₋₄ alkyl groups, C₁₋₄ haloalkyl groups, C₁₋₄ alkoxy groups, C₃₋₄ cycloalkyl groups and, —R′R″ groups, wherein R′ is chosen from a hydrogen atom and C₁₋₄ alkyl groups and R″ is chosen from a hydrogen atom and C₁₋₄ alkyl groups optionally substituted by a hydroxy group;
pyridinone groups substituted with one or more substituents chosen from halogen atoms, C₁₋₄ alkyl groups and C₁₋₄ haloalkyl groups; and
groups of formula: 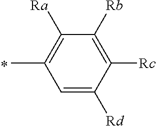
wherein:
Ra is chosen from a hydrogen atom and C₁₋₄ alkyl groups,
Rb is chosen from a hydrogen atom, halogen atoms and C₁₋₄ alkyl groups,
Rd is chosen from a hydrogen atom, C₁₋₄ alkyl groups and C₃₋₄ cycloalkyl groups,
Rc is chosen from a hydroxy group; C₁₋₄ alkoxy groups optionally substituted with one or more substituents chosen from hydroxy groups, C₁₋₃ alkoxy groups, hydroxycarbonyl groups, C₁₋₄ alkoxycarbonyl groups and NHR⁴ groups, wherein R⁴ represents a hydrogen atom; or
Rc is chosen from C₂₋₄ acyl groups and C₁₋₄ alkyl groups optionally substituted by a hydroxycarbonyl group, or
Rc represents —(CH₂)₍₀₋₄₎-L-R⁵ wherein L is chosen from —C(O)O—, —C(O)NH—, —S(O)₂NH—, —NH—, —CONHS(O)₂— and groups of formula: 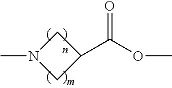
wherein n and m are each independently 1 or 2, and R⁵ is chosen from a hydrogen atom and C₁₋₄ alkyl groups optionally substituted by a hydroxycarbonyl group;
R² is chosen from:
monocyclic and bicyclic C₅₋₁₀ aryl groups, wherein the monocyclic and bicyclic groups are each independently optionally substituted with one or more substituents chosen from halogen atoms, C₁₋₄ alkyl groups, C₁₋₄ alkoxy groups and phenyl groups, wherein the alkyl groups are optionally substituted by one or more halogen atoms,
monocyclic and bicyclic 5-10 membered heteroaryl groups, wherein the monocyclic and bicyclic groups each independently comprise one or more heteroatoms chosen from N, S and O wherein the monocyclic and bicyclic groups are each independently optionally substituted with one or more substituents chosen from halogen atoms, C₁₋₄ alkyl groups, and C₁₋₄ alkoxy groups, wherein the C₁₋₄ alkyl groups are optionally substituted by one or more halogen atoms,
a dihydrobenzodioxine group, and
benzyl groups optionally substituted with one or more substituents chosen from halogen atoms,
and
R³ is chosen from:
linear and branched C₁₋₆ alkyl groups, C₃₋₆ cycloalkyl-C₁₋₄ alkyl groups, C₁₋₄ alkoxy-C₁₋₄ alkyl groups, di-alkylamino-C₁₋₄alkyl groups, and phenyl-C₁₋₄alkyl groups;
or a pharmaceutically acceptable salt thereof or a N-oxide thereof;
with the proviso that when Rc represents a methoxy or ethoxy group, then one of Rb or Rd can not be a hydrogen atom; and
with the additional proviso that the compound of formula (I) is not N-methyl-N-(5-(6-methylpyridin-3-yl)-1,3,4-thiadiazol-2-yl)nicotinamide, nor N-methyl-N-(5-(6-methylpyridin-3-yl)-1,3,4-thiadiazol-2-yl)isonicotinamide.

2. The compound according to claim 1, wherein R⁵ is chosen from a hydrogen atom and linear and branched C₁₋₄ alkyl groups, wherein the linear and branched C₁₋₄ alkyl groups are each independently optionally substituted by a hydroxycarbonyl group.

3. The compound according to claim 1, wherein R¹ is chosen from an imidazo[1,2-a]pyridyl group; pyridyl groups substituted with one or more substituents chosen from halogen atoms, C₁₋₄ alkyl groups, and C₁₋₄ alkoxy groups; pyridinone groups substituted with a chlorine atom; and groups of formula: 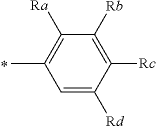
wherein:
Ra is chosen from a hydrogen atom and a methyl group,
Rb is chosen from a hydrogen atom, halogen atoms and C₁₋₄ alkyl groups,
Rd is chosen from a hydrogen atom and C₁₋₄ alkyl groups,
Rc is chosen from a hydroxy group, C₁₋₄ alkoxy groups optionally substituted with one or more substituents chosen from hydroxy groups, and C₁₋₃ alkoxy groups, or Rc represents —(CH₂)₍₀₋₂₎-L-R⁵, wherein L is chosen from —C(O)NH—, —NH—, groups of formula: 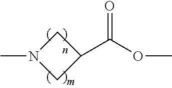
wherein n and m are each independently 1 or 2, and R⁵ is chosen from a hydrogen atom and C₁₋₄ alkyl groups optionally substituted by a hydroxycarbonyl group.

4. The compound according to claim 1, wherein R¹ is chosen from pyridyl groups substituted with one or two substituents chosen from halogen atoms, C₁₋₄ alkyl groups and C₁₋₄ alkoxy groups; and groups of formula: 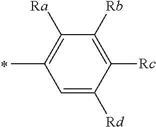
wherein:
Ra represents a hydrogen atom,
Rb is chosen from a hydrogen atom and C₁₋₄ alkyl groups,
Rd is chosen from a hydrogen atom and C₁₋₄ alkyl groups,
Rc is chosen from a hydroxy group, and C₁₋₄ alkoxy groups optionally substituted by one or more hydroxy groups, or
Rc represents —(CH₂)₍₀₋₂₎-L-R⁵, wherein L is chosen from —C(O)NH—, —NH—, and groups of formula: 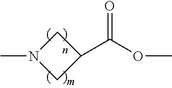
wherein n and m are each independently 1 or 2, and R⁵ is chosen from a hydrogen atom and C₁₋₄ alkyl groups optionally substituted by a hydroxycarbonyl group.

5. The compound according to claim 4, wherein R¹ is chosen from pyridyl groups substituted with one or two substituents chosen from chlorine atoms, methyl and methoxy groups; and groups of formula: 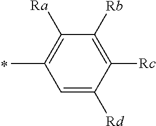
wherein:
Ra represents a hydrogen atom,
Rd represents a hydrogen atom or a methyl group,
Rb represents a hydrogen atom or a methyl group,
Rc is chosen from a hydroxy group, C₁₋₄ alkoxy groups optionally substituted by one or more substituents chosen from hydroxy groups; or
Rc represents —(CH₂)₍₀₋₁₎-L-R⁵, wherein L is chosen from —C(O)NH—, —NH—, and groups of formula: 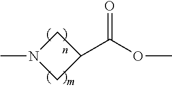
wherein both n and m have a value of 1, and R⁵ is chosen from a hydrogen atom and C₁₋₂ alkyl groups optionally substituted by a hydroxycarbonyl group.

6. The compound according to claim 1, wherein R² is chosen from phenyl groups substituted by one substituent chosen from halogen atoms, C₁₋₄ alkyl groups and C₁₋₄ alkoxy groups, wherein the alkyl groups are optionally substituted by one or more halogen atoms; and monocyclic N-containing 5-10 membered heteroaryl groups substituted by a halogen atom.

7. The compound according to claim 6, wherein R² is chosen from phenyl groups substituted with one substituent chosen from a fluorine atom, a chlorine atom, a methyl group, a trifluoromethyl group, and a methoxy group; and pyridyl groups substituted with a fluorine atom.

8. The compound according to claim 7, wherein R² is chosen from phenyl groups substituted with one substituent chosen from a fluorine atom, a chlorine atom and a methoxy group.

9. The compound according to claim 1, wherein R³ is chosen from linear C₃₋₆ alkyl groups and C₃₋₄ cycloalkyl-C₁₋₂ alkyl groups.

10. The compound according to claim 9, wherein R³ is chosen from a propyl group, a butyl group and cyclopropylmethyl groups.

11. The compound according to claim 10, wherein R³ represents a butyl group or a cyclopropylmethyl group.

12. The compound according to claim 1, wherein R¹ is chosen from pyridyl groups substituted with one or two substituents chosen from chlorine atoms, methyl and methoxy groups; and groups of formula: 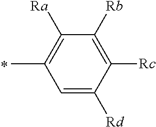
wherein:
Ra represents a hydrogen atom,
Rb represents a hydrogen atom or a methyl group,
Rd represents a hydrogen atom or a methyl group,
Rc is chosen from a hydroxy group, C₁₋₄ alkoxy groups optionally substituted with one or more substituents chosen from hydroxy groups; or
Rc represents —(CH₂)₍₀₋₁₎-L-R⁵, wherein L is chosen from —C(O)NH—, —NH—, and groups of formula: 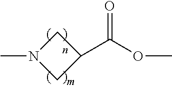
wherein n and m each have a value of 1, and R⁵ is chosen from a hydrogen atom and C₁₋₄ alkyl groups optionally substituted by a hydroxycarbonyl group;
R² is chosen from phenyl groups substituted with one substituent chosen from a fluorine atom, a chlorine atom and a methoxy group;
and R³ represents a butyl or a cyclopropylmethyl group.

13. The compound according to claim 1, wherein R¹ represents a group of formula: 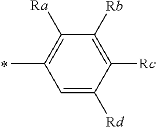
wherein:
Ra represents a hydrogen atom,
Rb is chosen from a hydrogen atom and C₁₋₄ alkyl groups,
Rd is chosen from a hydrogen atom and C₁₋₄ alkyl groups,
Rc is chosen from C₁₋₄ alkoxy groups optionally substituted with one or more substituents chosen from hydroxy groups and —NH₂ groups, or
Rc represents —(CH₂)₍₀₋₁₎-L-R⁵, wherein L is chosen from groups of formula: 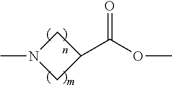
wherein n and m each have a value of 2, and R⁵ is chosen from a hydrogen atom and C₁₋₄ alkyl groups optionally substituted by a hydroxycarbonyl group;
R² is chosen from phenyl groups substituted with one or two substituents chosen from a fluorine atom and a chlorine atom;
and R³ represents a butyl or a cyclopropylmethyl group.

14. The compound according to claim 1, wherein R¹ is chosen from:
an imidazo[1,2-a]pyridyl group;
pyridyl groups substituted with one or two substituents chosen from chlorine atoms, methyl groups and methoxy groups;
pyridone groups optionally substituted with a chlorine atom or a methyl group; and
groups of formula: 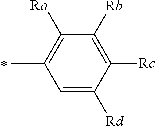
wherein
Ra represents a hydrogen atom or a methyl group,
Rb represents a hydrogen atom, a chlorine atom or a methyl group,
Rd represents a hydrogen atom or a methyl group,
Rc is chosen from a hydroxy group; C₁₋₄ alkoxy groups optionally substituted with one or more substituents chosen from hydroxy groups, methoxy groups, hydroxycarbonyl groups, C₁₋₄ alkoxycarbonyl groups and —NH₂ groups; or
Rc is chosen from —(CH₂)₍₀₋₂₎-L-R⁵, wherein L is chosen from —CO(O)—, —C(O)NH—, —S(O)₂NH—, —NH— and groups of formula: 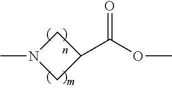
wherein n and m are each independently 1 or 2, and R⁵ is chosen from a hydrogen atom and linear and branched C₁₋₃ alkyl groups optionally substituted by a hydroxycarbonyl group;
R² is chosen from:
phenyl and naphthyl groups, wherein the phenyl and naphthyl groups are independently optionally substituted with one or two substituents chosen from chlorine atoms, fluorine atoms, methyl groups, trifluoromethyl groups, C₁₋₄ alkoxy groups and phenyl groups;
pyridine groups optionally substituted with a fluorine atom;
a dihydrobenzodioxine group; and a benzyl group; and
R³ is chosen from linear and branched C₁₋₅ alkyl, cyclopropylmethyl, methoxypropyl, diethylaminopropyl phenylethyl groups.

15. The compound according to claim 1, chosen from:
N-{5-[4-(aminosulfonyl)phenyl]-1,3,4-thiadiazol-2-yl}-N-butyl-3-methoxy-benzamide,
N-{5-[4-(aminosulfonyl)phenyl]-1,3,4-thiadiazol-2-yl}-N-butyl-2-naphthamide,
N-{5-[4-(aminosulfonyl)phenyl]-1,3,4-thiadiazol-2-yl}-N-butylbiphenyl-4-carboxamide,
N-{5-[4-(aminosulfonyl)phenyl]-1,3,4-thiadiazol-2-yl}-4-butoxy-N-butylbenzamide,
N-{5-[4-(aminosulfonyl)phenyl]-1,3,4-thiadiazol-2-yl}-N-butylbenzamide,
3-(4-(5-(N-butyl-3-methoxybenzamido)-1,3,4-thiadiazol-2-yl)phenyl)propanoic acid,
N-butyl-2-chloro-N-[5-(4-methoxy-3,5-dimethylphenyl)-1,3,4-thiadiazol-2-yl]benzamide,
N-butyl-2-chloro-N-[5-(4-hydroxy-3,5-dimethylphenyl)-1,3,4-thiadiazol-2-yl]benzamide,
N-butyl-2-fluoro-N-[5-(4-methoxy-3,5-dimethylphenyl)-1,3,4-thiadiazol-2-yl]benzamide,
N-Butyl-2-fluoro-N-[5-(4-hydroxy-3,5-dimethylphenyl)-1,3,4-thiadiazol-2-yl]benzamide,
N-butyl-N-[5-(4-methoxy-3,5-dimethylphenyl)-1,3,4-thiadiazol-2-yl]benzamide,
N-butyl-3-methoxy-N-[5-(4-methoxy-3,5-dimethylphenyl)-1,3,4-thiadiazol-2-yl]benzamide,
N-butyl-N-[5-(4-methoxy-3,5-dimethylphenyl)-1,3,4-thiadiazol-2-yl]-1-naphthamide,
N-butyl-2,6-dichloro-N-(5-(4-methoxy-3,5-dimethylphenyl)-1,3,4-thiadiazol-2-yl)benzamide,
N-butyl-N-[5-(4-methoxy-3,5-dimethylphenyl)-1,3,4-thiadiazol-2-yl]-2-(trifluoromethyl)benzamide,
N-butyl-N-[5-(4-methoxy-3,5-dimethylphenyl)-1,3,4-thiadiazol-2-yl]-2-phenylacetamide,
N-butyl-N-[5-(4-methoxy-3,5-dimethylphenyl)-1,3,4-thiadiazol-2-yl]-2-naphthamide,
N-butyl-N-[5-(4-methoxy-3,5-dimethylphenyl)-1,3,4-thiadiazol-2-yl]-2,3-dihydro-1,4-benzodioxine-6-carboxamide,
N-butyl-2-methoxy-N-[5-(4-methoxy-3,5-dimethylphenyl)-1,3,4-thiadiazol-2-yl]benzamide,
N-Butyl-3-fluoro-N-[5-(4-methoxy-3,5-dimethylphenyl)-1,3,4-thiadiazol-2-yl]benzamide,
N-Butyl-N-[5-(4-methoxy-3,5-dimethylphenyl)-1,3,4-thiadiazol-2-yl]nicotinamide,
N-Butyl-N-[5-(4-methoxy-3,5-dimethylphenyl)-1,3,4-thiadiazol-2-yl]pyridine-2-carboxamide,
N-Butyl-6-fluoro-N-[5-(4-methoxy-3,5-dimethylphenyl)-1,3,4-thiadiazol-2-yl]pyridine-2-carboxamide,
N-butyl-N-[5-(4-methoxy-3,5-dimethylphenyl)-1,3,4-thiadiazol-2-yl]-2-methylbenzamide,
2-chloro-N-[5-(4-methoxy-3,5-dimethylphenyl)-1,3,4-thiadiazol-2-yl]-Nmethylbenzamide,
N-butyl-2-chloro-N-[5-(4-methoxy-3-methylphenyl)-1,3,4-thiadiazol-2-yl]benzamide,
N-butyl-2-chloro-N-[5-(3-chloro-4-methoxyphenyl)-1,3,4-thiadiazol-2-yl]benzamide,
Methyl 4-{5-[butyl(2-chlorobenzoyl)amino]-1,3,4-thiadiazol-2-yl}benzoate
4-{5-[Butyl(2-chlorobenzoyl)amino]-1,3,4-thiadiazol-2-yl}benzoic acid,
N-Butyl-2-chloro-N-{5-[4-(2,3-dihydroxypropoxy)-3,5-dimethylphenyl]-1,3,4-thiadiazol-2-yl}benzamide,
N-Butyl-2-chloro-N-[5-(2-chloro-6-methoxypyridin-4-yl)-1,3,4-thiadiazol-2-yl]benzamide,
N-butyl-2-chloro-N-{5-[4-(2-methoxyethoxy)-3,5-dimethylphenyl]-1,3,4-thiadiazol-2-yl}benzamide,
2-Chloro-N-[5-(4-methoxy-3,5-dimethylphenyl)-1,3,4-thiadiazol-2-yl]-N-(2-methoxyethyl)benzamide,
2-Chloro-N-ethyl-N-[5-(4-methoxy-3,5-dimethylphenyl)-1,3,4-thiadiazol-2-yl]benzamide,
tert-Butyl (4-{5-[butyl(2-chlorobenzoyl)amino]-1,3,4-thiadiazol-2-yl}-2,6-dimethyl-phenoxy)acetate,
(4-{5-[Butyl(2-chlorobenzoyl)amino]-1,3,4-thiadiazol-2-yl}-2,6-dimethylphenoxy)acetic acid,
2-Chloro-N-[5-(4-methoxy-3,5-dimethylphenyl)-1,3,4-thiadiazol-2-yl]-N-(3-methylbutyl)benzamide,
2-Chloro-N-[3-(diethylamino)propyl]-N-[5-(4-methoxy-3,5-dimethylphenyl)-1,3,4-thiadiazol-2-yl]benzamide,
N-Butyl-2-chloro-N-[5-(2-chloro-6-methylpyridin-4-yl)-1,3,4-thiadiazol-2-yl]benzamide,
N-Butyl-2-chloro-N-[5-(6-chloro-2-oxo-1,2-dihydropyridin-4-yl)-1,3,4-thiadiazol-2-yl]benzamide,
N-Butyl-N-[5-(2-chloro-6-methoxypyridin-4-yl)-1,3,4-thiadiazol-2-yl]-2-fluorobenzamide,
N-Butyl-N-[5-(2-chloro-6-methylpyridin-4-yl)-1,3,4-thiadiazol-2-yl]-2-fluorobenzamide,
2-fluoro-N-[5-(4-methoxy-3,5-dimethylphenyl)-1,3,4-thiadiazol-2-yl]-N-propylbenzamide,
N-Butyl-2-fluoro-N-[5-(2-methylpyridin-4-yl)-1,3,4-thiadiazol-2-yl]benzamide,
N-butyl-2-fluoro-N-[5-(2-methoxypyridin-4-yl)-1,3,4-thiadiazol-2-yl]benzamide,
N-(cyclopropylmethyl)-2-fluoro-N-[5-(4-methoxy-3,5-dimethylphenyl)-1,3,4-thiadiazol-2-yl]benzamide,
3-(4-{5-[butyl(2-fluorobenzoyl)amino]-1,3,4-thiadiazol-2-yl}-2,6-dimethyl-phenyl)propanoic acid,
N-butyl-2-fluoro-N-(5-(6-methoxypyridin-3-yl)-1,3,4-thiadiazol-2-yl)benzamide,
N-butyl-2-fluoro-N-(5-(imidazo[1,2-a]pyridin-6-yl)-1,3,4-thiadiazol-2-yl)benzamide,
N-butyl-2-fluoro-N-(5-(imidazo[1,2-a]pyridin-7-yl)-1,3,4-thiadiazol-2-yl)benzamide
3-(4-(5-(N-butyl-2-chlorobenzamido)-1,3,4-thiadiazol-2-yl)benzamido)propanoic acid,
Ethyl 3-(4-(5-(N-butyl-2-chlorobenzamido)-1,3,4-thiadiazol-2-yl)benzamido)propanoate
N-butyl-N-(5-(4-carbamoylphenyl)-1,3,4-thiadiazol-2-yl)-2-chlorobenzamide,
1-(4-(5-(N-butyl-2-fluorobenzamido)-1,3,4-thiadiazol-2-yl)benzyl)azetidine-3-carboxylic acid,
(R)—N-butyl-N-(5-(4-(2,3-dihydroxypropoxy)-3,5-dimethylphenyl)-1,3,4-thiadiazol-2-yl)-2-fluorobenzamide,
2-fluoro-N-(5-(4-methoxy-3,5-dimethylphenyl)-1,3,4-thiadiazol-2-yl)-N-phenethylbenzamide,
2-(4-(5-(N-butyl-2-fluorobenzamido)-1,3,4-thiadiazol-2-yl)benzamido)acetic acid,
N-butyl-2-fluoro-N-[5-(1-methyl-2-oxo-1,2-dihydropyridin-4-yl)-1,3,4-thiadiazol-2-yl]benzamide,
N-(4-{5-[butyl(2-fluorobenzoyl)amino]-1,3,4-thiadiazol-2-yl}benzyl)-beta-alanine,
N-butyl-2-fluoro-N-[5-(1-methyl-6-oxo-1,6-dihydropyridin-3-yl)-1,3,4-thiadiazol-2-yl]benzamide,
N-butyl-2-fluoro-N-[5-(6-oxo-1,6-dihydropyridin-3-yl)-1,3,4-thiadiazol-2-yl]benzamide
3-(4-{5-[ethyl(2-fluorobenzoyl)amino]-1,3,4-thiadiazol-2-yl}-2,6-dimethylphenyl)propanoic acid,
N-butyl-N-[5-(4-{[(2S)-2,3-dihydroxypropyl]oxy}-3,5-dimethylphenyl)-1,3,4-thiadiazol-2-yl]-2-fluorobenzamide,
1-(4-{5-[butyl(2-fluorobenzoyl)amino]-1,3,4-thiadiazol-2-yl}benzyl)piperidine-4-carboxylic acid,
1-(4-{5-[butyl(2-methoxybenzoyl)amino]-1,3,4-thiadiazol-2-yl}benzyl)azetidine-3-carboxylic acid,
N-(cyclopropylmethyl)-N-[5-(4-{[(2R)-2,3-dihydroxypropyl]oxy}-3,5-dimethylphenyl)-1,3,4-thiadiazol-2-yl]-2-fluorobenzamide,
1-(4-{5-[butyl(pyridin-3-ylcarbonyl)amino]-1,3,4-thiadiazol-2-yl}benzyl)azetidine-3-carboxylic acid,
1-(4-{5-[ethyl(2-fluorobenzoyl)amino]-1,3,4-thiadiazol-2-yl}benzyl)azetidine-3-carboxylic acid,
N-(4-{5-[butyl(2-fluorobenzoyl)amino]-1,3,4-thiadiazol-2-yl}benzoyl)-beta-alanine,
N-(cyclopropylmethyl)-N-[5-(4-{[(2S)-2,3-dihydroxypropyl]oxy}-3,5-dimethylphenyl)-1,3,4-thiadiazol-2-yl]-2-fluorobenzamide,
1-(4-{5-[(2-fluorobenzoyl)(propyl)amino]-1,3,4-thiadiazol-2-yl}benzyl)azetidine-3-carboxylic acid,
(3R)-3-[(4-{5-[butyl(2-fluorobenzoyl)amino]-1,3,4-thiadiazol-2-yl}benzoyl)amino]-butanoic acid,
(3S)-3-[(4-{5-[butyl(2-fluorobenzoyl)amino]-1,3,4-thiadiazol-2-yl}benzoyl)amino]-butanoic acid,
N-{5-[4-(aminomethyl)phenyl]-1,3,4-thiadiazol-2-yl}-N-butyl-2-fluorobenzamide,
1-[2-(4-{5-[butyl(2-fluorobenzoyl)amino]-1,3,4-thiadiazol-2-yl}phenyl)ethyl]azetidine-3-carboxylic acid,
1-(4-{5-[(cyclopropylmethyl)(2-fluorobenzoyl)amino]-1,3,4-thiadiazol-2-yl}benzyl)-azetidine-3-carboxylic acid,
1-(4-{5-[(2-fluorobenzoyl)(3-methylbutyl)amino]-1,3,4-thiadiazol-2-yl}benzyl)-azetidine-3-carboxylic acid,
1-(4-{5-[(2-fluorobenzoyl)(methyl)amino]-1,3,4-thiadiazol-2-yl}benzyl)azetidine-3-carboxylic acid,
1-(4-{5-[butyl(2-fluorobenzoyl)amino]-1,3,4-thiadiazol-2-yl}-3-methylbenzyl)-azetidine-3-carboxylic acid,
4-[(4-{5-[butyl(2-fluorobenzoyl)amino]-1,3,4-thiadiazol-2-yl}benzoyl)amino]butanoic acid,
1-(4-{5-[(2-fluorobenzoyl)(2-methoxyethyl)amino]-1,3,4-thiadiazol-2-yl}benzyl)-azetidine-3-carboxylic acid,
1-(4-{5-[butyl(phenylacetyl)amino]-1,3,4-thiadiazol-2-yl}benzyl)azetidine-3-carboxylic acid,
1-(4-{5-[butyl(2,6-difluorobenzoyl)amino]-1,3,4-thiadiazol-2-yl}benzyl)azetidine-3-carboxylic acid,
N-(4-{5-[butyl(phenylacetyl)amino]-1,3,4-thiadiazol-2-yl}benzyl)-beta-alanine,
N-(4-{5-[butyl(2-fluorobenzoyl)amino]-1,3,4-thiadiazol-2-yl}benzyl)glycine
ethyl 1-(4-{5-[butyl(2-fluorobenzoyl)amino]-1,3,4-thiadiazol-2-yl}benzyl)azetidine-3-carboxylate,
1-[4-(5-{butyl[(2-fluorophenyl)acetyl]amino}-1,3,4-thiadiazol-2-yl)benzyl]azetidine-3-carboxylic acid,
1-(4-{5-[(cyclopropylmethyl)(2-methylbenzoyl)amino]-1,3,4-thiadiazol-2-yl}benzyl)-azetidine-3-carboxylic acid,
1-(4-{5-[(cyclopropylmethyl)(phenylacetyl)amino]-1,3,4-thiadiazol-2-yl}benzyl)-azetidine-3-carboxylic acid,
1-(4-{5-[(cyclopropylmethyl)(4-methoxybenzoyl)amino]-1,3,4-thiadiazol-2-yl}benzyl)azetidine-3-carboxylic acid,
1-(4-{5-[benzoyl(butyl)amino]-1,3,4-thiadiazol-2-yl}benzyl)azetidine-3-carboxylic acid,
1-(4-{5-[butyl(2-fluorobenzoyl)amino]-1,3,4-thiadiazol-2-yl}-2-methylbenzyl)-azetidine-3-carboxylic acid,
1-(4-{5-[butyl(2-chlorobenzoyl)amino]-1,3,4-thiadiazol-2-yl}benzyl)azetidine-3-carboxylic acid,
1-(4-{5-[ethyl(phenylacetyl)amino]-1,3,4-thiadiazol-2-yl}benzyl)azetidine-3-carboxylic acid,
1-(4-{5-[ethyl(2-methoxybenzoyl)amino]-1,3,4-thiadiazol-2-yl}benzyl)azetidine-3-carboxylic acid,
1-(4-{5-[(2-chlorobenzoyl)(ethyl)amino]-1,3,4-thiadiazol-2-yl}benzyl)azetidine-3-carboxylic acid,
1-(4-{5-[butyl(2-fluorobenzoyl)amino]-1,3,4-thiadiazol-2-yl}-3-methylbenzyl)-pyrrolidine-3-carboxylic acid,
1-(4-{5-[benzoyl(ethyl)amino]-1,3,4-thiadiazol-2-yl}benzyl)azetidine-3-carboxylic acid,
1-(4-{5-[butyl(2-fluorobenzoyl)amino]-1,3,4-thiadiazol-2-yl}benzyl)pyrrolidine-3-carboxylic acid,
1-(4-{5-[butyl(phenylacetyl)amino]-1,3,4-thiadiazol-2-yl}-3-methylbenzyl)azetidine-3-carboxylic acid,
1-[4-(5-{butyl[(2-chlorophenyl)acetyl]amino}-1,3,4-thiadiazol-2-yl)-3-methylbenzyl]-azetidine-3-carboxylic acid,
1-(4-{5-[ethyl(3-fluorobenzoyl)amino]-1,3,4-thiadiazol-2-yl}benzyl)azetidine-3-carboxylic acid,
1-[4-(5-{butyl[(2-chlorophenyl)acetyl]amino}-1,3,4-thiadiazol-2-yl)-3-methylbenzyl]-pyrrolidine-3-carboxylic acid,
1-(4-{5-[butyl(2-chlorobenzoyl)amino]-1,3,4-thiadiazol-2-yl}-3-methylbenzyl)-azetidine-3-carboxylic acid,
N-{5-[4-(2-aminoethoxy)-3,5-dimethylphenyl]-1,3,4-thiadiazol-2-yl}-N-butyl-2-fluorobenzamide,
1-(4-{5-[butyl(2-fluorobenzoyl)amino]-1,3,4-thiadiazol-2-yl}-2,6-dimethylbenzyl)-azetidine-3-carboxylic acid,
N-butyl-N-{5-[4-(2,3-dihydroxypropoxy)-3,5-dimethylphenyl]-1,3,4-thiadiazol-2-yl}-2-methylbenzamide,
1-(4-{5-[(3-chloro-2-fluorobenzoyl)(ethyl)amino]-1,3,4-thiadiazol-2-yl}benzyl)-azetidine-3-carboxylic acid,
1-(4-{5-[butyl(3-chloro-2-fluorobenzoyl)amino]-1,3,4-thiadiazol-2-yl}-3-methylbenzyl)piperidine-4-carboxylic acid,
1-(4-{5-[butyl(3-chloro-2-fluorobenzoyl)amino]-1,3,4-thiadiazol-2-yl}-3-methylbenzyl)azetidine-3-carboxylic acid,
N-butyl-3-chloro-N-{5-[4-(2,3-dihydroxypropoxy)-3,5-dimethylphenyl]-1,3,4-thiadiazol-2-yl}-2-fluorobenzamide,
1-(4-{5-[(3-chloro-2-fluorobenzoyl)(ethyl)amino]-1,3,4-thiadiazol-2-yl}benzyl)-piperidine-4-carboxylic acid,
N-{5-[4-(3-amino-2-hydroxypropoxy)-3,5-dimethylphenyl]-1,3,4-thiadiazol-2-yl}-N-butyl-2-fluorobenzamide,
1-(4-{5-[(3-chloro-2-fluorobenzoyl)(cyclopropylmethyl)amino]-1,3,4-thiadiazol-2-yl}benzyl)piperidine-4-carboxylic acid,
1-(4-{5-[butyl(3-chloro-2-fluorobenzoyl)amino]-1,3,4-thiadiazol-2-yl}-2,6-dimethyl-benzyl)azetidine-3-carboxylic acid,
1-(4-{5-[butyl(3-chloro-2-fluorobenzoyl)amino]-1,3,4-thiadiazol-2-yl}-2,6-dimethylbenzyl)piperidine-4-carboxylic acid,
1-(4-{5-[butyl(2-chlorobenzoyl)amino]-1,3,4-thiadiazol-2-yl}-2,6-dimethyl-benzyl)azetidine-3-carboxylic acid,
1-(4-{5-[butyl(2-chlorobenzoyl)amino]-1,3,4-thiadiazol-2-yl}-3-methylbenzyl)-piperidine-4-carboxylic acid,
1-(4-{5-[butyl(2-chlorobenzoyl)amino]-1,3,4-thiadiazol-2-yl}-2,6-dimethylbenzyl)-piperidine-4-carboxylic acid, and
or a pharmaceutically acceptable salt or N-oxide thereof.

16. (canceled)

17. A method for treating a pathological condition or disease susceptible to amelioration by sphingosine-1-phosphate receptors (S1P1) agonists, wherein the method comprises administering an effective amount of a compound according to claim 1.

18. The method according to claim 17, wherein the pathological condition or disease is chosen from autoimmune diseases, chronic immune and inflammatory diseases, transplant rejection, malignant neoplastic diseases, angiogenic-related disorders, pain, neurological diseases, viral and infectious diseases.

19. The method according to claim 18, wherein the pathological condition or disease is chosen from multiple sclerosis, transplant rejection, systemic lupus erythematosus, asthma, psoriasis, rheumatoid arthritis, psoriatic arthritis and Crohn's disease,

20. A pharmaceutical composition comprising a compound as claimed in claim 1, and a pharmaceutically acceptable diluent or carrier.

21-22. (canceled)

23. A composition comprising:
(i) a compound according to claim 1; and
(ii) at least one compound chosen from:
a) Beta interferons,
b) Immunomodulators,
c) Inhibitors of DNA synthesis and repair,
d) Anti-alpha 4 integrin antibodies,
e) Alpha 4 integrin antagonists,
f) Dihydrofolate reductase inhibitors,
g) Glucocorticoids,
h) DHODH inhibitors,
i) Fumaric acid esters,
j) Immunomodulators,
k) Anti-CD20 monoclonal antibodies,
l) Anti-CD52,
m) Anti-CD25,
n) Anti-CD88,
o) Calcineurin inhibitors,
p) IMPDH inhibitors,
q) Cannabinoid receptor agonists,
r) Chemokine CCR1 antagonists,
s) Chemokine CCR2 antagonists,
t) Interferon alpha,
u) NF-kappaB activation inhibitors,
v) JAK inhibitors,
w) Syk inhibitors,
x) PKC inhibitors,
y) Phosphosdiesterase IV inhibitors,
z) P38 Inhibitors, and
aa) MEK inhibitors.
